(19) United States
(12) Patent Application Publication
(10) Pub. No.: US 2023/0355511 A1
(43) Pub. Date: Nov. 9, 2023

(54) BIODEGRADABLE POLYMER AND SOLVENT COMPOSITIONS AND SYSTEMS FOR EXTENDED STORAGE AND DELIVERY OF ACTIVE PHARMACEUTICAL INGREDIENTS

(71) Applicant: TOLMAR INTERNATIONAL LIMITED, Dublin (IE)

(72) Inventors: Garrett GLOVER, Fort Collins, CO (US); Gregory FIELDSON, Fort Collins, CO (US)

(21) Appl. No.: 18/247,222

(22) PCT Filed: Sep. 20, 2021
(86) PCT No.: PCT/IB2021/058557

§ 371 Date: Mar. 29, 2023

Related U.S. Application Data
(60) Provisional application No. 63/085,860, filed on Sep. 30, 2020.

(51) Int. Cl. A61K 9/00; A61K 47/34; A61K 45/06

(52) U.S. Cl. CPC A61K 9/0024; A61K 47/34; A61K 45/06

(57) ABSTRACT

Pharmaceutical compositions comprising a biodegradable polymer, a solvent system, and an active pharmaceutical ingredient suitable for extended storage and delivery of the active pharmaceutical ingredient contained therein are disclosed, along with methods of making and using the same.

### FIELD

This application pertains to the field of pharmaceutical compositions comprising a biodegradable polymer, a solvent, and an active pharmaceutical ingredient suitable for extended storage and delivery of the active pharmaceutical ingredient contained therein.

### BACKGROUND

Flowable, polymer-containing compositions useful as biodegradable controlled release formulations for medicinal substances or active pharmaceutical ingredients (APIs) are described, for instance, in U.S. Pat. Nos. 6,143,314; 6,565,874; 8,470,359; 8,187,640; and WO2017/024027. One type of controlled release formulation composition includes a biodegradable polymer or copolymer, one or more biocompatible organic solvent(s), and an API suspended or dispersed therein. These compositions are administered in a flowable, preferably liquid state to the patient, typically via a syringe needle.

APIs possess different inherent physicochemical properties due to their molecular structure and composition which will influence how they behave when prepared as a mixture within any given media. Depending upon the media of choice, an API may form a monophasic mixture (e.g., a solution) or a biphasic mixture (e.g., a suspension or a dispersion). Maintaining the homogeneity of APIs within a biphasic suspension mixture, for example within a polymeric formulation, during long-term storage is a significant challenge in the development of polymeric pharmaceutical delivery systems as useful pharmaceutical products. Because the API tends to separate from the polymeric formulation and distribute unevenly within it, the composition must be remixed prior to administration to a subject. In some cases, the homogeneity of the pharmaceutical composition may only be partially restored even after extensive remixing. Thus, there is a need in the art for polymeric pharmaceutical delivery compositions and systems in which the API remains homogeneously suspended in the polymeric formulation under long-term storage conditions.

### SUMMARY

In a first aspect, the present disclosure provides a pharmaceutical extended release composition comprising an active pharmaceutical ingredient (API) and a biocompatible polymer-solvent system comprising a biodegradable polymer and a solvent system comprising at least one solvent and at least one component that modifies the melting point of the polymer-solvent system to: (i) form a highly viscous composition that maintains a substantially homogeneous distribution of the API at a first temperature from about 0° C. to about 8° C.; and (ii) form a flowable composition suitable for administration by injection at a second temperature from about 18° C. to about 25° C.

In some embodiments, the first temperature is from about 2° C. to about 6° C.

In some embodiments, the second temperature is from about 20° C. to about 24° C.

In some embodiments, the biocompatible polymer-solvent system comprises one or more solvents selected from the group consisting of amides, acids, alcohols, esters of monobasic acids, ether alcohols, sulfoxides, lactones, polyhydroxy alcohols, esters of polyhydroxy alcohols, ketones, and ethers.

In some embodiments, the biocompatible polymer-solvent system comprises one or more solvents selected from the group consisting of N-methyl-2-pyrrolidone (NMP), acetone, cyrene, butyrolactone, ε-caprolactone, N-cycylohexyl-2-pyrrolidone, diethylene glycol monomethyl ether, dimethylacetamide, dimethyl formamide, dimethyl sulfoxide (DMSO), ethyl acetate, ethyl lactate, N-ethyl-2-pyrrolidone, glycerol formal, glycofurol, N-hydroxyethyl-2-pyrrolidone, isopropylidene glycerol, lactic acid, methoxypolyethylene glycol, methoxypropyleneglycol, methyl acetate, methyl ethyl ketone, methyl lactate, benzyl benzoate (BnBzO), polysorbate 80, polysorbate 60, polysorbate 40, polysorbate 20, polyoxyl 35, polyethylene glycol (PEG), hydrogenated castor oil, polyoxyl 40 hydrogenated castor oil, sorbitan monolaurate, sorbitan monostearate, sorbitan monooleate, benzyl alcohol, n-propanol, isopropanol, tert-butanol, propylene glycol, 2-pyrrolidone, α-tocopherol, triacetin, tributyl citrate, acetyl tributyl citrate, acetyl triethyl citrate, triethyl citrate, esters thereof, and combinations thereof.

In some embodiments, the biocompatible polymer-solvent system comprises one or more solvents selected from the group consisting of N-methyl-2-pyrrolidone (NMP), dimethyl sulfoxide (DMSO), benzyl benzoate (BnBzO), and combinations thereof.

In some embodiments, the solvent system of the biocompatible polymer-solvent system comprises at least 1 solvent and a co-solvent selected from a low molecular weight polyethylene glycol (PEG) having a number average molecular weight of PEG 300, PEG 400, or a combination thereof.

In some embodiments, the low molecular weight polyethylene glycol (PEG) is selected from the group consisting of PEG 300, PEG 400, or a combination thereof does not modify the melting point of the biocompatible polymer-solvent system.

In some embodiments, the at least one component of the solvent system comprises at least one low molecular weight polyethylene glycol (PEG) having a number average molecular weight of at least 500.

In some embodiments, the at least one low molecular weight PEG is selected from the group consisting of PEG 500, PEG 600, PEG 1000, PEG 1450, PEG 3350, and combinations thereof.

In some embodiments, a weight percent (wt %) ratio of the at least one low molecular weight PEG to the biocompatible polymer-solvent system is from about 1:20 to about 20:1.

In some embodiments, the composition comprises from about 1 wt % to about 90 wt % of the low molecular weight PEG.

In some embodiments, the composition comprises from about 15 wt % to about 70 wt % of PEG 600.

In some embodiments, the composition comprises from about 4 wt % to about 45 wt % of PEG 1000.

In some embodiments, the composition comprises from about 2 wt % to about 35 wt % of PEG 1450.

In some embodiments, the composition comprises from about 1 wt % to about 10 wt % of PEG 3350.

In some embodiments, the biodegradable polymer is selected from the group consisting of polylactic acid, polyglycolic acid, polylactide, polyglycolide, polycaprolactones, polyanhydrides, polyamides, polyurethanes, polyesteramides, polyorthoesters, polydioxanones, polyacetals, polyketals, polycarbonates, polyphosphazenes, polyhydroxybutyrates, polyhydroxyvalerates, polyalkylene oxalates, polyalkylene succinates, poly(malic acid), polyethylene glycol, hyaluronic acid, chitin and chitosan, a copolymer thereof, a terpolymer thereof, and any combination thereof.

In some embodiments, the biodegradable polymer comprises monomers that are selected from the group consisting of lactide, glycolide, caprolactone, p-dioxanone, trimethylene carbonate, 1,5-dioxepan-2-one, 1,4-dioxepan-2-one, ethylene oxide, propylene oxide, sebacic anhydride, diketene acetals/diols, lactic acid, and combinations thereof.

In some embodiments, the biodegradable polymer comprises lactide and glycolide monomer residues.

In some embodiments, a molar ratio of the lactide to glycolide monomer residues is from about 45:55 to about 99:1.

In some other embodiments, a molar ratio of the lactide to glycolide monomer residues is from about 50:50 to about 90:10.

In some embodiments, the biodegradable polymer comprises lactide and/or glycolide monomer residues, and monomer residues selected from the group consisting of E-caprolactone, trimethylene carbonate, and combinations thereof.

In some embodiments, a molar ratio of lactide and/or glycolide monomer residues to the ε-caprolactone and/or trimethylene carbonate monomer residues is from about 10:90 to about 90:10.

In other embodiments, a molar ratio of lactide and/or glycolide monomer residues to the E-caprolactone and/or trimethylene carbonate monomer residues is from about 25:75 to about 75:25.

In yet other embodiments, a molar ratio of lactide monomer residues to E-caprolactone and/or trimethylene carbonate monomer residues is about 75:25.

In some embodiments, the biodegradable polymer comprises at least one carboxylic acid end group and the biodegradable polymer is synthesized by initiation with an organic acid.

In some embodiments, the biodegradable polymer comprises at least one hydroxy end group and the biodegradable polymer is synthesized by initiation with a mono functional alcohol.

In some embodiments, the biodegradable polymer comprises at least one hydroxy end group and is substantially free of terminal carboxy end groups, and the biodegradable polymer is synthesized by initiation with a diol.

In some embodiments, the biodegradable polymer has an average molecular weight from about 1 kDa to about 100 kDa.

In some other embodiments, the biodegradable polymer has an average molecular weight from about 1 kDa to about 60 kDa.

In some embodiments, the biodegradable polymer is not soluble in water.

In some embodiments, the composition comprises from about 0.1 wt % to about 70 wt % of the biodegradable polymer.

In other embodiments, the composition comprises from about 1 wt % to about 70 wt % of the biodegradable polymer.

In yet other embodiments, the composition comprises from about 10 wt % to about 50 wt % of the biodegradable polymer.

In some embodiments, the API is in the form of a liquid or a finely divided solid that is suspended and/or dispersed in the composition.

In some embodiments, the API is one or more of a small molecule, a peptide, or a polypeptide.

In some embodiments, the API is in a form of a base, an ester, a hydrate, a solvate, a salt, or a prodrug.

In some embodiments, the API is present in the composition in a dosage effective for greater than one week.

In other embodiments, the API is present in the composition in a dosage effective for greater than one month.

In other embodiments, the API is present in the composition in a dosage effective for greater than three months.

In other embodiments, the API is present in the composition in a dosage effective for greater than six months.

In other embodiments, the API is present in the composition in a dosage effective for greater than twelve months.

In some embodiments, the composition is suitable for administration by autoinjection at a temperature of about 18° C. or more.

In some embodiments, the composition is suitable for administration by injection through an 18 to 26 gauge needle at a temperature of about 18° C. or more.

In some embodiments, the composition has a viscosity of about 20,000 cP or more at the first temperature.

In other embodiments, the composition has a viscosity of about 10,000 cP or more at the first temperature.

In yet other embodiments, the composition has a viscosity of about 5,000 cP or more at the first temperature.

In some embodiments, the composition has a viscosity of about 20,000 cP or less at the second temperature.

In other embodiments, the composition has a viscosity of about 10,000 cP or less at the second temperature.

In yet other embodiments, the composition has a viscosity of about 5,000 cP or less at the second temperature.

In some embodiments, the composition maintains a substantially homogeneous distribution of the API within the composition when stored at the first temperature for at least 6 months or longer.

In some embodiments, the composition maintains a substantially homogeneous distribution of the API within the composition when stored at the first temperature for at least 12 months or longer.

In some embodiments, the composition maintains a substantially homogeneous distribution of the API within the composition when stored at the first temperature for at least 24 months or longer.

In some embodiments, the composition maintains a substantially homogeneous distribution of the API within the composition when stored at the first temperature for at least 36 months or longer.

In some embodiments, the composition is a liquid-liquid dispersion.

In other embodiments, the liquid-liquid dispersion is a liquid-in-oil dispersion.

In yet other embodiments, the composition is an emulsion.

In some embodiments, the composition is stored at the first temperature and then warmed to the second temperature prior to administering to a subject.

In some embodiments, the composition does not require mixing prior to administering to a subject.

In other embodiments, the composition is mixed for five mixing cycles or less prior to administering to a subject.

In some embodiments, upon contact of the composition with a bodily fluid, a solvent dissipates from the composition and an in situ liquid or solid implant forms.

In some embodiments, the in situ liquid or solid implant releases the API over an extended time period of at least about 1 week or longer.

In some embodiments, the in situ liquid or solid implant releases the API over an extended time period of at least about 2 weeks or longer.

In some embodiments, the in situ liquid or solid implant releases the API over an extended time period of at least about 1 month or longer.

In other embodiments, the in situ liquid or solid implant releases the API over an extended time period of at least about 3 months or longer.

In yet other embodiments, the in situ liquid or solid implant releases the API over an extended time period of at least about 6 months or longer.

In a second aspect, the present disclosure provides a method of treating a subject with an API, comprising administering to the subject the pharmaceutical composition described herein.

In a third aspect, the present disclosure provides a delivery system for administration of a pharmaceutical composition, comprising a syringe and the pharmaceutical composition described herein, wherein the pharmaceutical composition is contained within the syringe.

In some embodiments, the syringe comprises an 18 to 26 gauge needle.

In some embodiments, the syringe is contained in an autoinjector.

### BRIEF DESCRIPTION OF THE DRAWINGS

FIG. 1 shows a TU-liquid polymer formulation, comprising by weight 20% TU, 30% of a 14 kDa 75:25 PDLCL-acid-initiated polymer, 25% NMP, and 25% PEG 300, where the TU particles have separated from the liquid polymer phase upon centrifugation at 2,000 rpm for 25 minutes at 20° C. with the syringe tip pointed outward, i.e., away from the axis of rotation. A known property of suspensions and/or dispersions, i.e., any mixture comprising more than a single phase, is that the phases will inherently separate over time or can be forced to separate by applying an external force. Centrifugation was used herein to simulate long term storage of the suspension.

FIG. 2 shows the dosage inhomogeneity present within the beginning (left, light grey), middle (medium grey), and end (right, dark grey) fractions regions of a syringe (relative to the syringe tip) containing a TU-liquid polymer formulation, comprising by weight 20% TU, 30% of a 14 kDa 75:25 PDLCL-acid-initiated polymer, 25% NMP, and 25% PEG 300, after having undergone centrifugation to separate the TU particles from the liquid polymer phase. Equivalent samples were then remixed using 0.5, 5, 10, 15, or 25 mixing cycles. These samples were then likewise analyzed to assess if the dosage homogeneity was restored via the mixing cycles. The error bars represent the standard deviation for three replicate samples.

FIG. 3 shows a DSC melt profile for a solidifying liquid polymer formulation comprising by weight 37.5% of a 5 kDa 75:25 PDLCL-acid-initiated polymer, 9.3% NMP, 9.3% PEG 300, and 43.9% PEG 600. The slopes associated with the area under the curve (AUC) are used to calculate the Melt Onset Temperature and the Peak Melt Temperature, which are at 3.88° C. and 16.05° C., respectively.

FIG. 4A shows the physical state transition temperatures, Melt Onset Temperatures (° C.) and Melt Peak Temperatures (° C.), obtained from DSC for solidifying liquid polymer formulations comprising 37.5% of a 5 kDa 75:25 PDLCL-acid-initiated polymer and varying amounts of NMP, PEG 300, PEG 400, and PEG 600 (0). The compositions for the solidifying formulations are provided in Table 1. FIG. 4B shows the Melt Onset Temperatures (♦) and Melt Peak Temperatures (▪) for the same liquid polymer formulations in FIG. 4A as a function of wt % of PEG 600 in the formulation.

FIG. 5 shows the dosage inhomogeneity present within the beginning (left, light grey), middle (middle, medium grey), and end (right, dark grey) regions of syringes (relative to the syringe tip) containing either a non-solidifying or a solidifying TU-liquid polymer formulation. The control non-solidifying formulation (shown on the far-left), comprises by weight 20% TU, 30% of a 14 kDa 75:25 PDLCL-acid-initiated polymer, 25% NMP, and 25% PEG 300. The control formulation was cooled to 4° C. and then centrifuged at 2,000 rpm for 60 minutes prior to analyzing the TU dose homogeneity via fractionating the syringe contents into beginning, middle, and end regions for analysis. The solidifying formulation comprises by weight 20% TU, 30% of a 5 kDa 75:25 PDLCL-acid-initiated polymer, 7.5% NMP, 7.5% PEG 300, and 35% PEG 600. This formulation was cooled to 4° C. prior to syringe fractionation analysis (shown on the middle-left), Similarly, this formulation was likewise cooled to 4° C. then centrifuged at 2,000 rpm for 60 minutes prior to syringe fractionation analysis (shown on the middle-right). Lastly, this formulation was subjected to 5 freeze/thaw cycles and then centrifuged at 2,000 rpm for 60 minutes prior to syringe fractionation analysis (shown on the far-right). The error bars represent the standard deviation for three replicate samples.

FIG. 6A shows the physical state transition temperatures, Melt Onset Temperatures (° C.) versus and Melt Peak Temperatures (° C.), obtained from DSC for solidifying solid polymer formulations comprising either 34% or 37.5% of a 56 kDa 50:50 PLG-acid-initiated polymer and varying amounts of NMP, PEG 300, and PEG 600 (0). The compositions for the solidifying formulations are provided in Table 2. FIG. 6B shows the Melt Onset Temperatures (└) and Melt Peak Temperatures (▪) for the same solid polymer formulations in FIG. 6A as a function of wt % of PEG 600 in the formulation.

FIG. 7 shows DSC melt profiles of solidifying solid polymer formulations comprising varying amounts of 56 kDa 50:50 PLG-acid-initiated solid polymer, NMP, and PEG 600. The compositions for the solidifying formulations are provided in Table 3. The top melt profile corresponds to a reference solidifying liquid polymer formulation comprising, a 37.5% of a 5 kDa 75:25 PDLCL glycolic acid-initiated polymer, 9.3% NMP, 9.3% PEG 300, and 43.9% PEG 600.

FIG. 8 shows the viscosities (cP) of solid polymer formulations as a function of the amount of PEG 600 (mg) added to a pre-formulated composition comprising by weight 34% of a 56 kDa 50:50 PLG-acid-initiated solid polymer and 66% NMP at 5° C. (o) and 25° C. (⋅). The compositions of the final solid polymer, NWP, and PEG-600 formulations are provided in Table 3.

FIG. 9 shows DSC melt profiles for various low molecular weight PEGs: PEG 300, PEG 400, PEG 600, PEG 1000, and PEG 1450.

FIG. 10 shows DSC melt profiles for solidifying solid polymer formulations comprising varying amounts of 56 kDa 50:50 PLG-acid-initiated solid polymer, NMP, and PEG 1000. The compositions are provided in Table 4. The top melt profile corresponds to a reference solidifying liquid polymer formulation comprising, by weight, 37.5% of a 5 kDa 75:25 PDLCL glycolic acid-initiated polymer, 9.3% NMP, 9.3% PEG 300, and 43.9% PEG 600.

FIG. 11 shows DSC melt profiles for solidifying solid polymer formulations comprising varying amounts of a 56 kDa 50:50 PLG-acid-initiated solid polymer, NMP, and PEG 1450. The compositions are provided in Table 5. The top melt profile corresponds to a reference solidifying liquid polymer formulation comprising by weight 37.5% of a 5 kDa 75:25 PDLCL glycolic acid-initiated polymer, 9.3% NMP, 9.3% PEG 300, and 43.9% PEG 600.

FIG. 12 shows DSC melt profiles for solidifying solid polymer formulation comprising varying amounts of 56 kDa 50:50 PLG-acid-initiated solid polymer, NMP, and PEG 3350. The compositions are provided in Table 6.

FIG. 13A shows the physical state transition temperatures, Melt Onset Temperatures (° C.) versus and Melt Peak Temperatures (° C.) for solidifying solid polymer formulations comprising varying amounts of a 25 kDa 50:50 PLG-hexanediol-initiated polymer, NMP, PEG 300, and PEG 600 (⋅). The compositions are provided in Table 7. FIG. 13B shows the Melt Onset Temperatures (♦) and Melt Peak Temperatures (▪) for the same solid polymer formulations in FIG. 13A as a function of wt % of PEG 600 in the formulation.

FIG. 14 shows the time release profiles at 37° C. of testosterone undecanoate (TU) from TU-liquid polymer formulations comprising by weight 20% TU, 30% of a 14.2 kDa 75:25 PDLCL-acid-initiated polymer, and varying amounts of NMP, PEG 600, and PEG 300: (▴)15% NMP and 35% PEG 600; (o) 7.5% NMP, 7.5% PEG 300, and 35% PEG 600; and (▪) a non-solidifying control formulation comprising 25% NMP and 25% PEG 300. Also provided is the time release profiles for a non-formulated TU sample with a particle size of 67 μm (⋅).

FIG. 15 shows the time release profiles at 37° C. of testosterone cypionate (TC) from TC-liquid polymer formulations comprising by weight 20% TC, 30% of a 14.2 kDa 75:25 PDLCL-acid-initiated polymer, and varying amounts of NMP, PEG 600, and PEG 300: (▴) 15% NMP and 35% PEG 600; (o) 7.5% NMP, 7.5% PEG 300, and 35% PEG 600; and (▪) a non-solidifying control formulation comprising 25% NMP, and 25% PEG 300. Also provided is the time release profiles for a non-formulated TC sample with a particle size of 41 μm (⋅).

FIG. 16 shows the time release profiles at 37° C. of testosterone cypionate (TC) from two liquid 75:25 PDLCL-acid-initiated polymer formulations with differing molecular weights comprising by weight 20% TC, 7.5% NMP, 7.5% PEG 300, 35% PEG 600, and 30% of a 10 kDa polymer (grey ♦) or 30% of a 14.2 kDa polymer (o). Also provided is the time release profile for a non-solidifying control sample (▪) comprising 20% TC, 30% of a 14.2 kDa 75:25 PDLCL-acid-initiated polymer, 25% NMP, and 25% PEG 300 and for a non-formulated TC sample with a particle size of 41 μm (⋅).

FIG. 17 shows the time release profiles at 37° C. of testosterone cypionate (TC) from two liquid 75:25 PDLCL-acid-initiated polymer formulations with differing molecular weights comprising by weight 20% TC, 15% NMP, 35% PEG 600, and 30% of a 14 kDa polymer (▴) or 30% of a 22 kDa polymer (grey ♦). Also provided is the time release profile for a non-solidifying control sample (▪) comprising 20% TC, 30% of a 14 kDa 75:25 PDLCL-acid-initiated polymer, 25% NMP, and 25% PEG 300 and for a non-formulated TC sample with a particle size of 41 μm (⋅).

FIG. 18A shows an image of a polymer-oil droplet dispersion for a liquid polymer-oil suspension formulation, comprising by weight 30% of a 14 kDa 75:25 PDLCL acid-initiated polymer, 25% NMP, 25% PEG 300, and 20% mineral oil, that was vigorously mixed at 25° C. via syringe-to-syringe prior to imaging. FIG. 18B shows an image of the same formulation in FIG. 18A after freezing the sample at 5° C. for two days before allowing the sample to warm without additional mixing of the sample prior to imaging (Note: the image is not omitted; instead, the lack of significant material within the field of vision gives the appearance of an omitted (i.e., blank) image). Both images were taken at 25° C., 10× magnification.

FIG. 19A shows an image of a polymer-oil droplet dispersion for a liquid polymer-oil suspension formulation, comprising by weight 30% of a 14 kDa 75:25 PDLCL acid-initiated polymer, 25% NMP, 25% PEG 600, and 20% mineral oil, that was vigorously mixed at 25° C. via syringe-to-syringe prior to imaging. FIG. 19B shows an image of the same formulation in FIG. 19A after freezing the sample at 5° C. for two days before allowing the sample to warm without additional mixing of the sample prior to imaging. Both images taken at 25° C., 10× magnification.

FIG. 20A shows an image of a polymer-oil droplet dispersion for a liquid polymer-oil suspension formulation, comprising by weight 30% of a 14 kDa 75:25 PDLCL acid-initiated polymer, 7.5% NMP, 7.5% PEG 300, 35% PEG 600, and 20% mineral oil, that was vigorously mixed at 25° C. via syringe-to-syringe prior to imaging. FIG. 20B shows an image of the same formulation in FIG. 20A after freezing the sample at 5° C. for two days before allowing the sample to warm without additional mixing of the sample prior to imaging. Both images were taken at 25° C., 10× magnification.

FIG. 21 shows the dosage inhomogeneity present within the beginning (left, light grey), middle (middle, medium grey), and end (right, dark grey) regions of a syringe (relative to the syringe tip) containing a LA-liquid polymer formulation, comprising by weight 12% LA, 30.8% of an 18.8 kDa 75:25 PDLCL-acid-initiated polymer, and 57.2% BnBzO, after having undergone centrifugation to separate the LA particles from the liquid polymer phase. Equivalent samples were then remixed using 0.5, 5, 10, 15, or 25 mixing cycles. These samples were then likewise analyzed to assess if the dosage homogeneity was restored via the mixing cycles. The error bars represent the standard deviation for three replicate samples.

FIG. 22 shows the dosage inhomogeneity present within the beginning (left, light grey), middle (middle, medium grey), and end (right, dark grey) fraction regions of syringes (relative to the syringe tip) containing either a non-solidifying or a solidifying LA-liquid polymer formulation. The control non-solidifying formulation (shown on the left), comprises by weight 12% LA, 30.8% of an 18.8 kDa 75:25 PDLCL-acid-initiated polymer, and 57.2% BnBzO. The control non-solidifying LA-liquid polymer formulation was cooled to 4° C. and then centrifuged at 2,000 rpm for 60 minutes prior to analyzing the LA dose homogeneity. The solidifying LA-liquid polymer formulation comprises by weight 12% LA, 18.5% of an 18.8 kDa 75:25 PDLCL-acid-initiated polymer, 34.3% BnBzO, and 35.2% PEG 600. This solidifying LA-liquid polymer formulation was cooled to 4° C. prior to syringe fractionation analysis without centrifugation (shown on the middle). Separately, this solidifying LA-liquid polymer formulation was likewise cooled to 4° C. and then centrifuged at 2,000 rpm for 60 minutes prior to syringe fractionation analysis (shown on the right). The error bars represent the standard deviation for three replicate samples.

FIG. 23 shows the dosage inhomogeneity present within the beginning (left, light grey), middle (middle, medium grey), and end (right, dark grey) fraction regions of syringes (relative to the syringe tip) containing either a non-solidifying or a solidifying LA-liquid polymer formulation. The control non-solidifying LA-liquid polymer formulation (shown on the left), comprises by weight 12% LA, 30.8% of an 18.8 kDa 75:25 PDLCL-acid-initiated polymer, and 57.2% BnBzO. The control non-solidifying LA-liquid polymer formulation was cooled to 4° C. and then centrifuged at 2,000 rpm for 60 minutes prior to analyzing the LA dose homogeneity in the syringe fractions. The solidifying LA-liquid polymer formulation comprises by weight 12% LA, 29.3% of an 18.8 kDa 75:25 PDLCL-acid-initiated polymer, 54.3% BnBzO, and 4.4% PEG 1000. This solidifying LA-liquid polymer formulation was cooled to 4° C. prior to syringe fractionation analysis (shown in the middle). Separately, this solidifying LA-liquid polymer formulation was likewise cooled to 4° C. and then centrifuged at 2,000 rpm for 60 minutes prior to syringe fractionation analysis (shown on the right). The error bars represent the standard deviation for three replicate samples.

FIG. 24 shows the dosage inhomogeneity present within the beginning (left, light grey), middle (middle, medium grey), and end (right, dark grey) regions of syringes (relative to the syringe tip) containing either a non-solidifying or a solidifying LA-liquid polymer formulation. The control non-solidifying LA-liquid polymer formulation (shown on the left), comprises by weight 12% LA, 30.8% of an 18.8 kDa 75:25 PDLCL-acid-initiated polymer, and 57.2% BnBzO. The control non-solidifying LA-liquid polymer formulation was cooled to 4° C. and then centrifuged at 2,000 rpm for 60 minutes prior to analyzing the LA dose homogeneity in the syringe fractions. The solidifying LA-liquid polymer formulation comprises by weight 12% LA, 24.6% of an 18.8 kDa 75:25 PDLCL-acid-initiated polymer, 45.8% BnBzO, and 17.6% PEG 1000. This solidifying LA-liquid polymer formulation was cooled to 4° C. prior to syringe fractionation analysis (shown in the middle). Separately, this solidifying LA-liquid polymer formulation was likewise cooled to 4° C. and then centrifuged at 2,000 rpm for 60 minutes prior to syringe fractionation analysis (shown on the right). The error bars represent the standard deviation for three replicate samples.

### DETAILED DESCRIPTION

Described herein are extended release pharmaceutical compositions or formulations comprising an active pharmaceutical ingredient (API), a biodegradable polymer and a biocompatible solvent system suitable for long-term storage prior to administration to a subject or patient.

In embodiments, the present disclosure provides an extended release pharmaceutical composition comprising an API and a biocompatible polymer-solvent system comprising a biodegradable polymer and at least one component that modifies the melting point of the polymer-solvent system. In some embodiments, the pharmaceutical composition or formulation forms or is a highly viscous composition or formulation that maintains a substantially homogeneous distribution of the API at a temperature from about 0° C. to about 8° C. and forms or is a flowable composition or formulation suitable for administration by injection at temperatures from about 18° C. to about 25° C. Accordingly, the pharmaceutical composition may be stored at a temperature from about 0° C. to about 8° C., referred to herein as “refrigeration” or “cold storage” temperatures, for extended periods of time and maintain a substantially homogeneous distribution of the API therein. The composition may be warmed to temperatures from about 18° C. to about 25° C., referred to herein as “room temperature” or “administration temperatures”, prior to administration, to form a flowable composition that may be administered to a subject via injection with syringes or needles. In some instances, the composition may be warmed to about at least 25° C. or greater. The changes made to the original formulation composition and/or the process of cooling and storing the pharmaceutical compositions such that the composition forms a highly viscous composition at a temperature from about 0° C. to about 8° C. and the subsequent formation of a flowable composition suitable for administration by injection at temperatures from about 18° C. to about 25° C., does not significantly impact the drug release profile of the formulation, which is verified using an in vitro release test (IVRT) model as a comparative study.

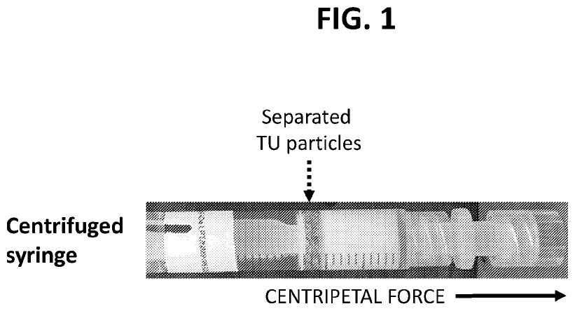

In various embodiments, the API may be suspended, dispersed, or otherwise inter-mixed with the polymer-solvent system to form a mixture. In some embodiments, the resulting API-polymer-solvent mixture may comprise at least two or more distinct phases. In some embodiments, the resulting API-polymer-solvent mixture forms a highly viscous, liquid, semi-solid, or solid composition that maintains a substantially homogeneous distribution of the API at a temperature from about 0° C. to about 8° C., and that also forms a flowable composition suitable for administration by injection at temperatures from about 18° C. to about 25° C. In some embodiments, the API may remain immobilized within the highly viscous composition at refrigeration temperatures. In other words, at a temperature from about 0° C. to about 8° C., the highly viscous composition prevents the API from migrating or separating from the polymer-solvent system. In contrast, API-polymer-solvent systems that do not form a highly viscous composition upon cooling to refrigeration temperature may experience API separation from the polymer-solvent system when the composition is stored for an extended period of time and/or when subjected to an external force as shown in FIG. 1. The formation of a highly viscous composition at refrigeration temperatures allows the pharmaceutical composition to be stored for extended periods of time, e.g., up to about 36 months or longer, without there being a substantial change in the API dosage uniformity within or throughout the composition.

In some embodiments, the API may be substantially immobilized within the polymer-solvent system when the composition comprises a viscosity such that the composition is essentially solidified at a given first temperature. Furthermore, in such instances, the viscosity of the composition may be flowable at a second temperature of interest. In some cases, the viscosity of the composition may be sufficient such as to: 1) prevent API movement at a first temperature, 2) solidify the composition at a first temperature, 3) not solidify the composition at a second temperature, 4) be flowable at the second temperature, and 5) be suitable for injection when at the second temperature.

Alternatively, in certain other embodiments, the API may be substantially immobilized within the polymer-solvent system when the composition comprises a viscosity such that the composition is not solidified at a given first temperature. Furthermore, in such instances, the viscosity of the composition may be flowable at a second temperature of interest. In some cases, the viscosity of the composition may be sufficient such as to: 1) prevent API movement at a first temperature, 2) not solidify the composition at a first temperature, 3) not solidify the composition at a second temperature, 4) be flowable at the second temperature, and 5) be suitable for injection when at the second temperature.

In some embodiments, the viscosity of the composition may depend on several factors, including but not limited to, the type of polymer, the amount of polymer in the composition, the polymer monomer ratio (e.g. the ratio of lactide:glycolide), the type of solvent, the amount of solvent, the type of API, the API particle size, temperature, etc. In some embodiments, the viscosity of the composition may be greater than about 0 cP, about 500 cP, about 1,000 cP, about 2,000 cP, about 3,000 cP, about 4,000 cP, about 5,000 cP, about 6,000 cP, about 7,000 cP, about 8,000 cP, about 9,000 cP, about 10,000 cP, about 11,000 cP, about 12,000 cP, about 13,000 cP, about 14,000 cP, about 15,000 cP, about 16,000 cP, about 17,000 cP, about 18,000 cP, about 19,000 cP, about 20,000 cP, about 30,000 cP, about 40,000 cP, about 50,000 cP, about 60,000 cP, about 70,000 cP, about 80,000 cP, about 90,000 cP about 100,000 cP, about 110,000 cP, about 120,000 cP, about 130,000 cP, about 140,000 cP, about 150,000 cP, about 160,000 cP, about 170,000 cP, about 180,000 cP, about 190,000 cP, about 200,000 cP, or higher than about 200,000 cP. In some instances, the viscosity of the composition may be any whole number from about 1,000 to about 200,000 cP. In yet other instances, the viscosity of the composition may be any whole number from about 1,000 to about 20,000 cP. In yet other instances, the viscosity of the composition may be any whole number from about 20,000 to about 200,000 cP.

As used herein, the term “highly viscous” means that the viscosity of the composition is any viscosity wherein the API is substantially immobilized within the polymer-solvent system, such that a substantially homogeneous dosage uniformity within the composition is maintained. In some embodiments, the composition may comprise a viscosity wherein the API is substantially immobilized within the polymer-solvent system. In some embodiments, the pharmaceutical composition may form or may be a highly viscous composition at refrigeration or cold storage temperatures, typically ranging from about 0° C. to about 8° C., about 2° C. to about 6° C., or about 3° C. to about 5° C. In some embodiments, the pharmaceutical composition forms a highly viscous composition at a temperature of about 0° C., about 1° C., about 2° C., about 3° C., about 4° C., about 5° C., about 6° C., about 7° C., or about 8° C. In some embodiments, the pharmaceutical composition may form or may be a highly viscous composition at a temperature that is at any tenth of a degree from about 0° C. to about 8° C. In some embodiments, the pharmaceutical composition may form or may be a highly viscous composition at a temperature of about 0° C. In some embodiments, the pharmaceutical composition may form or may be a highly viscous composition at a temperature of about 2° C. In some embodiments, the pharmaceutical composition may form or may be a highly viscous composition at a temperature of about 4° C. In some embodiments, the pharmaceutical composition may form or may be a highly viscous composition at a temperature of about 6° C. In some embodiments, the pharmaceutical composition may form or may be a highly viscous composition at a temperature of about 8° C. When the pharmaceutical composition is subsequently warmed to about room temperature or administration temperatures, e.g., from about 18° C. to about 25° C., after being cooled to form a highly viscous composition, the viscosity of the pharmaceutical composition decreases sufficiently enough such that the composition may become sufficiently flowable to be administered to a patient via a syringe or needle. As used herein, the term “flowable” means that the pharmaceutical composition may be administered by injection though a syringe with a 6 to 32 or larger gauge needle or, in other cases, may be administered by injection using an auto-injector. In some embodiments, the pharmaceutical composition may be administered by injection into a patient by injection though a syringe with a 6 to 32 or larger gauge needle. For clarity, the larger the needle gauge size, the smaller the needle diameter. In other embodiments, the pharmaceutical composition may be administered by injection into a patient by injection though a syringe with an 18 to 30 gauge needle. In other embodiments, the pharmaceutical composition may be administered by injection into a patient by injection though a syringe with an 18 to 24 gauge needle. In some embodiments, the pharmaceutical composition may be administered into a patient by subcutaneous injection though a syringe with a 25 to 30 gauge needle. In some embodiments, the pharmaceutical composition may be administered into a patient by intramuscular injection though a syringe with a 20 to 25 gauge needle. In some embodiments, the pharmaceutical composition may be administered to a patient by injection using an auto-injector.

In some embodiments, the pharmaceutical composition may form or may be a flowable composition when at about room temperature or administration temperatures. In some embodiments, the pharmaceutical composition may form or may be a flowable composition at a temperature from about 18° C. to about 25° C. In some embodiments, the pharmaceutical composition may form or may be a flowable composition at a temperature from about 20° C. to 23° C. In some embodiments, the pharmaceutical composition may form or may be a flowable composition at about 18° C., about 19° C., about 20° C., about 21° C., about 22° C., about 23° C., about 24° C., or about 25° C. In some embodiments, the pharmaceutical composition may form or may be a flowable composition at a temperature that is about any tenth of a degree from about 18° C. to about 25° C. In some embodiments, the pharmaceutical composition may form or may be a flowable composition at about 20° C. In some embodiments, the pharmaceutical composition may form or may be a flowable composition at about 22° C. In some other embodiments, the pharmaceutical composition may form or may be a flowable composition at temperatures above 8° C.

In some embodiments, the viscosity of the flowable composition may be less than about 500 cP, less than about 1,000 cP, less than about 2,000 cP, less than about 3,000 cP, less than about 4,000 cP, less than about 5,000 cP, less than about 6,000 cP, less than about 7,000 cP, less than about 8,000 cP, less than about 9,000 cP, less than about 10,000 cP, less than about 11,000 cP, less than about 12,000 cP, less than about 13,000 cP, less than about 14,000 cP, less than about 15,000 cP, less than about 16,000 cP, less than about 17,000 cP, less than about 18,000 cP, less than about 19,000 cP, or less than about 20,000 cP. Alternatively, the viscosity of the flowable composition may less than any whole number from about 500 to about 20,000 cP.

In another aspect, when the pharmaceutical composition is warmed in preparation for administration to a patient, the dose uniformity of the API may be substantially uniform within the pharmaceutical composition. In some embodiments, the pharmaceutical composition does not need to be re-mixed, agitated, or otherwise disturbed to restore the dosage uniformity prior to being administered to a patient. In other embodiments, the pharmaceutical compositions may need to be slightly re-mixed, agitated, or otherwise disturbed in order to restore the dosage uniformity prior to being administered to a patient. In such embodiments, the pharmaceutical composition may be mixed via a single mixing cycle to restore the dosage uniformity prior to being administered to a patient. In yet other another embodiment, the pharmaceutical composition may be mixed five or less mixing cycles to restore the dosage uniformity prior to being administered to a patient. In this context, the formulations may be re-mixed by transferring the formulation into a mixing syringe. Each mixing cycle comprises a full depression and retraction of the syringe plunger rod. Other methods of mixing include, but are not limited to, agitation or disturbances such as shaking, rocking, nutating, or inverting the syringe, may also be employed.

An aspect of the present disclosure is that the pharmaceutical composition maintains a substantially homogeneous distribution of the API within or throughout the formulation at refrigeration temperatures. As used herein, the phrase “substantially homogeneous” means that the dosage uniformity, as expressed herein, but not limited to, in percent relative standard deviation (% RSD), varies by no more than about ±6% RSD within or throughout the polymer-solvent system. In other words, as a non-limiting example, when the pharmaceutical composition is stored for an extended period of time, there may be the tendency for the API to separate from the polymer-solvent system. If this occurs, in some instances, the polymer may migrate to the bottom of the sample and the API may migrate towards the top of the sample. As such, in some embodiments, for pharmaceutical compositions of the present disclosure stored at a temperature from about 0° C. to about 8° C., the dosage uniformity may vary by no more than about ±6% RSD within the sample. In some embodiments, the dosage uniformity may vary by no more than about ±0.5% RSD, no more than about ±1% RSD, no more than about ±1.5% RSD, no more than about ±2% RSD, no more than about ±2.5% RSD, no more than about ±3% RSD, no more than about ±3.5% RSD, no more than about ±4% RSD, no more than about ±4.5% RSD, no more than about ±5% RSD, no more than about ±5.5% RSD, or no more than about ±6% RSD, or alternatively by no more than about any tenth of a percent from about ±0.5% RSD to about ±6% RSD. In some embodiments, the pharmaceutical composition may undergo at least one freeze/thaw cycle (i.e., the sample is cooled to refrigeration temperatures and then is warmed to room or administrative temperatures) and still maintain a substantially homogeneous distribution of the API within the formulation. In some embodiments, the pharmaceutical composition may undergo more than one freeze/thaw cycle and still maintain a substantially homogeneous distribution of the API within the formulation.

In some embodiments, the viscosity of the substantially homogeneous composition may be greater than about 500 cP, greater than about 1,000 cP, greater than about 2,000 cP, greater than about 3,000 cP, greater than about 4,000 cP, greater than about 5,000 cP, greater than about 6,000 cP, greater than about 7,000 cP, greater than about 8,000 cP, greater than about 9,000 cP, greater than about 10,000 cP, greater than about 11,000 cP, greater than about 12,000 cP, greater than about 13,000 cP, greater than about 14,000 cP, greater than about 15,000 cP, greater than about 16,000 cP, greater than about 17,000 cP, greater than about 18,000 cP, greater than about 19,000 cP, or greater than about 20,000 cP. In some further embodiments, the viscosity of the substantially homogeneous composition may be greater than about greater than about 30,000 cP, greater than about 40,000 cP, greater than about 50,000 cP, greater than about 60,000 cP, greater than about 70,000 cP, greater than about 80,000 cP, greater than about 90,000 cP, greater than about 100,000 cP, greater than about 110,000 cP, greater than about 120,000 cP, greater than about 130,000 cP, greater than about 140,000 cP, greater than about 150,000 cP, greater than about 160,000 cP, greater than about 170,000 cP, greater than about 180,000 cP, greater than about 190,000 cP, or even greater than about 200,000 cP. Alternatively, the viscosity of the substantially homogeneous composition may be greater than any whole number from about 500 to about 200,000 cP.

In some embodiments, the pharmaceutical compositions may maintain the dosage uniformity as a substantially homogeneous distribution of the API at a temperature from about 0° C. to about 8° C. for a storage time of at least 1 week or longer, at least 2 weeks or longer, at least about 1 month or longer, at least about 3 months or longer, at least about 6 months or longer, at least about 9 months or longer, at least about 12 months or longer, at least about 18 months or longer, at least 24 months or longer, at least about 30 months or longer, or at least about 36 months or longer. In other embodiments, the pharmaceutical compositions, when stored at a temperature from about 0° C. to about 8° C., may have a shelf life of at least about 1 month or longer, at least about 3 months or longer, at least about 6 months or longer, at least about 9 months or longer, at least about 12 months or longer, at least about 18 months or longer, at least about 24 months or longer, at least about 30 months or longer, or at least about 36 months or longer.

Another aspect of the disclosure is that the pharmaceutical composition may form or may be a highly viscous composition at a temperature from about 0° C. to about 8° C., which maintains a substantially homogeneous distribution of the API, and which may further form or may be a flowable composition suitable for administration by injection at a temperature from about 18° C. to about 25° C. In some embodiments, the pharmaceutical composition may form or may be a highly viscous composition that maintains a substantially homogeneous distribution of the API at a temperature of about 0° C., about 1° C., about 2° C., about 3° C., about 4° C., about 5° C., about 6° C., about 7° C., or about 8° C., and may also form or may also be a flowable composition suitable for administration by injection at a temperature of about 18° C., about 19° C., about 20° C., about 21° C., about 22° C., about 23° C., about 24° C., or about 25° C. As a non-limiting example, the pharmaceutical composition may form or may be a highly viscous composition that maintains a substantially homogeneous distribution of the API at a temperature from about 2° C. to about 6° C., and which may also form or may be a flowable composition suitable for administration by injection at a temperature from about 20° C. to about 23° C.

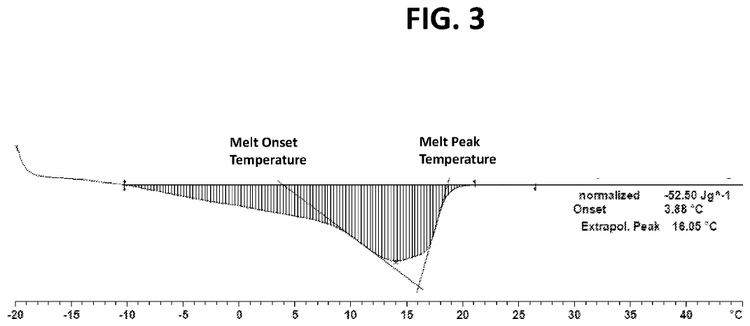

Differential Scanning calorimetry (DSC) thermal analysis may be used to determine the freezing and melting characteristics of the pharmaceutical composition of the present disclosure. The results from the DSC thermal analysis are depicted as a plot with a shaded area under the curve (AUC) representing the calorimetric difference between the sample (i.e., the polymer formulation) and the control (i.e., the pan) used within the calorimeter. This calorimetric difference is the amount of additional heat needed to melt the polymer formulation when compared to the control. The slopes associated with the AUC are used to calculate the rate and extent of physical state transitions, i.e., the Melt Onset Temperature and the Melt Peak Temperature. A representative DSC plot is shown in FIG. 3. In some embodiments, the physical state transitions that are determined by DSC may be used to determine if the pharmaceutical composition is suitable such that the composition may form or may be a highly viscous composition that maintains a substantially homogeneous distribution of the API at a temperature from about 0° C. to about 8° C. and may also form or may be a flowable composition suitable for administration by injection at temperatures from about 18° C. to about 25° C. In some embodiments, suitable pharmaceutical compositions may have a Melt Onset Peak Temperature of about 0° C. to about 14° C. and a Melt Peak Temperature of about 10° C. to about 24° C. In some embodiments, suitable pharmaceutical compositions may have a Melt Onset Peak Temperature of about 2° C. to about 12° C. and a Melt Peak Temperature of about 12° C. to about 22° C.

The present disclosure achieves the benefits discussed above by the addition of at least one component into the biocompatible polymer-solvent system that modifies the melting point of the biocompatible polymer-solvent system. The at least one component is selected such that the pharmaceutical composition forms a temperature sensitive high viscosity composition. This temperature sensitive high viscosity composition may become or may be highly viscous at temperatures of from about 0° C. to about 8° C. such that a substantially homogeneous distribution of an API included within the composition is maintained through immobilization of said API within the composition. In some embodiments, the high viscosity composition may form a semi-solid or solid composition. In other embodiments, the high viscosity composition may not form a semi-solid or solid composition. This temperature sensitive high viscosity composition then may become a flowable composition suitable for administration by injection when at temperatures from about 18° C. to about 25° C. In some embodiments, a single component that modifies the melting point of the biocompatible polymer-solvent system may be added to the biocompatible polymer-solvent system. While in other embodiments, two components that modify the melting point of the biocompatible polymer-solvent system may be added to the biocompatible polymer-solvent system. Similarly, in yet other embodiments, three or more components that modify the melting point of the biocompatible polymer-solvent system may be added to the biocompatible polymer-solvent system. In some embodiments, the at least one component may be a low molecular weight polyethylene glycol (PEG) having a number average molecular weight of at least 500 or higher. In some cases, the at least one low molecular weight PEG may be PEG 500 or greater, wherein the PEG may increase in molecular weight by an integer number value of 44 g/mol, which represents a single ethylene glycol (EG) monomer. In some instances, as non-limiting examples, the at least one low molecular weight PEG may be selected from the group consisting of PEG 500, PEG 600, PEG 1000, PEG 1450, PEG 3350, and combinations thereof.

### Definitions

As used herein, the term “animal” may refer to any organism of the kingdom Animalia. Examples of “animals” as that term is used herein include, but are not limited to, humans (Homo sapiens); companion animals, such as dogs, cats, and horses; and livestock animals, such as cows, goats, sheep, and pigs.

As used herein, the term “biocompatible” may mean “not harmful to living tissue.”

As used herein, the term “biodegradable” may refer to any water-insoluble material that is converted under physiological conditions into one or more water-soluble materials, without regard to any specific degradation mechanism or process.

As used herein, the term “co-solvent” may refer to a substance added to a solvent to increase or modify the solubility of a solute in the solvent.

As used herein, the term “dosage uniformity” may refer to the variation in concentration of the API at various locations or segments within the composition and/or the storage or delivery vehicle (e.g., a syringe). Unless otherwise specified, the dosage uniformity is expressed herein as the percent relative standard deviation (% RSD).

As used herein, the term “liquid” may refer to the ability of a composition to undergo deformation under a shearing stress, regardless of the presence or absence of a non-aqueous solvent. Liquid polymer compositions and the liquid polymers (also referred to as “liquid polymers) according to the present disclosure have a liquid physical state at ambient and body temperatures and remain liquid in vivo, i.e., in a largely aqueous environment. The liquid polymer compositions and liquid polymers have a definite volume, but are an amorphous, non-crystalline mass with no definite shape. In addition, the liquid polymers according to the present disclosure are not soluble in body fluid or water and therefore, after injection into the body and dissipation of the solvent, remain as a cohesive mass when injected into the body without themselves significantly dissipating. In addition, such liquid polymer compositions may have a viscosity, density, and flowability to allow delivery of the composition through standard gauge or small gauge needles (e.g., 6-32 gauge) with low to moderate injection force using standard syringes. The liquid polymers of the present disclosure are further characterized as not forming a solid implant in situ in the body when injected into the body as part of a sustained release drug delivery system that includes the liquid polymers and a biocompatible solvent. In other words, liquid polymers according to the present disclosure remain in a substantially liquid form in situ upon exposure to an aqueous environment, such as upon injection into the body, including after the solvent in the administered composition has dissipated. The liquid polymers of the present disclosure may be further characterized being non-crystalline, amorphous, non-thermoplastic, non-thermosetting, and/or non-solid. “Liquids,” as that term is used herein, may also exhibit viscoelastic behavior, i.e., both viscous and elastic characteristics when undergoing deformation, such as time-dependent and/or hysteretic strain. By way of non-limiting example, viscoelastic materials that are generally flowable but have a partially solid character and/or a plastic- or gel-like character, such as cake batter or raw pizza dough, and similar materials, are “liquids” as that term is used herein. In some embodiments, materials having a non-zero yield stress that do not deform at stresses below the yield stress, and that are readily deformable without a characteristic of material fracture or rupture at materials above the yield stress, may be “liquids” as that term is used herein.

As used herein, the terms “molecular weight” and “average molecular weight,” unless otherwise specified, may refer to a weight-average molecular weight as measured by a conventional gel permeation chromatography (GPC) instrument (such as an Agilent 1260 Infinity Quaternary LC with Agilent G1362A Refractive Index Detector) utilizing polystyrene standards and tetrahydrofuran (THF) as the solvent.

As used herein, the terms “patient” and “subject” may be considered interchangeable and may generally refer to an animal or a human to which a composition or formulation of the present disclosure is administered or is to be administered.

As used herein, the term “polymer” may generally refer to polymers, copolymers and/or terpolymers formed of repeating units, which can be linear, branched, grafted and/or star-shaped. Water-insoluble polymers that are converted under physiological conditions into one or more water-soluble materials are referred to as herein as “biodegradable polymers,”.

As used herein, the term “small molecule” may refer to an organic compound having a molecular weight less than 900 daltons (Da).

As used herein, the term “solvent” may refer to a liquid into which a solid or liquid substances may be suspended or dispersed.

Unless otherwise specified, all ratios between monomers in a copolymer disclosed herein are molar ratios.

Unless otherwise specified, various amounts of API, biodegradable polymer, and solvents and co-solvents are reported in weight percentages of the solvent system, solvent system and biodegradable polymer, or pharmaceutical composition.

Unless otherwise specified, all particle sizes and particle size distributions disclosed herein are determined according to volume-based particle size measurements, such as, by way of non-limiting example, by use of a laser diffraction particle size analyzer such as a Malvern Mastersizer® instrument. Software programs and calculations that can convert from a number-based distribution analysis to a volume-based distribution analysis (and vice versa) are well known in the art; therefore, for particle sizes calculated using a number-based method, a volume-based particle size can also be estimated. Volume-based particle size distribution measurements are the default choice for many ensemble light scattering particle size measurement techniques, including laser diffraction, and are generally used in the pharmaceutical industry.

### Biocompatible Polymer-Solvent System

The pharmaceutical composition according to the present disclosure comprises a biocompatible polymer-solvent system that includes a biodegradable polymer and a solvent system comprising at least one solvent and at least one component that modifies the melting point of the polymer-solvent system. The biocompatible polymer solvent system may comprise one or more solvents. The biocompatible polymer-solvent system may optionally comprise one or more co-solvents. The one or more solvents and/or one or more co-solvents and/or the at least one components may be collectively referred to as a solvent system.

In some embodiments, the polymer-solvent system composition may be a suspension. In some embodiments, the polymer-solvent system composition may be a dispersion. In some embodiments, the polymer-solvent system composition may be a liquid-liquid dispersion. As non-limiting examples, types of dispersions may include liquid-in oil, oil-in liquid, or oil-in-oil dispersions. In some embodiments, the polymer-solvent system composition may be an emulsion. In some embodiments, the polymer-solvent system composition may be a self-emulsifying emulsion (i.e., an emulsion that forms via chemical means rather than by physical means). In some embodiments, the polymer-solvent system composition may be any mixture comprising more than a single phase which will inherently separate over time or which can be forced to separate when an external force is applied.

Solvents and co-solvents suitable for use in embodiments of the present disclosure include, by way of non-limiting examples, acetone, benzyl benzoate (BnBzO), cyrene, butyrolactone, ε-caprolactone, CRODASOL™ (PEG-6 caprylic/capric glycerides and PEG-60 almond glycerides), N-cycylohexyl-2-pyrrolidone, diethylene glycol monomethyl ether, dimethyl acetamide, dimethyl formamide, dimethyl sulfoxide (DMSO), ethyl acetate, ethyl lactate, N-ethyl-2-pyrrolidone, glycerol formal, glycofurol, N-hydroxyethyl-2-pyrrolidone, isopropylidene glycerol, lactic acid, methoxypolyethylene glycol, methoxypropylene glycol, methyl acetate, methyl ethyl ketone, methyl lactate, N-methyl-2-pyrrolidone (NMP), low-molecular weight (MW) polyethylene glycol (PEG), polysorbate 80, polysorbate 60, polysorbate 40, polysorbate 20, polyoxyl 35 hydrogenated castor oil, polyoxyl 40 hydrogenated castor oil, sorbitan monolaurate, sorbitan monostearate, sorbitan monooleate, benzyl alcohol, isopropanol, tert-butanol, n-propanol, propylene glycol, 2-pyrrolidone, a-tocopherol, triacetin, tributyl citrate, acetyl tributyl citrate, acetyl triethyl citrate, triethyl citrate, esters thereof, and combinations thereof. In some embodiments, one or more of these and other solvents may form a suspension when provided in relatively small quantities and/or when used as a co-solvent or additive. In other embodiments, one or more of these and other solvents may form a solution when provided in relatively large quantities.

In some embodiments of the present disclosure, the biocompatible solvent system comprises one or more solvents selected from the group consisting of N-methyl-2-pyrrolidone (NMP), dimethyl sulfoxide (DMSO), benzyl benzoate (BnBzO), and CRODASOL™. In some embodiments, the biocompatible solvent system may further comprise one or more low molecular weight PEGs having a molecular weight of about 400 Da or less (e.g., PEG 300 or PEG 400) which may act as a co-solvent. In some embodiments, where the solvent system comprises one or more low molecular weight PEGs having a molecular weight of about 400 Da or less (e.g., PEG 300 or PEG 400), these low molecular weight PEGs do not modify the melting point of the biocompatible polymer-solvent system. In some embodiments, where the solvent system comprises one or more PEGs having a molecular weight of about 400 Da or less, such PEG may be included in an amount of about 5 wt % to about 35 wt % of the composition. In some further embodiments, the solvent system comprises one or more PEGs having a molecular weight of about 400 Da or less at about 5 wt % to about 15 wt % of the composition.

In some embodiments of the present disclosure, the biocompatible solvent system comprises N-methyl-2-pyrrolidone (NMP). In some embodiments, the biocompatible solvent system comprises NMP and one or more PEG having a number average molecular weight of about 400 Da or less (e.g., PEG 300 or PEG 400). In some embodiments where the solvent system comprises NMP, it may further comprise one or more PEGS having a number average molecular weight of about 400 Da or less, such that PEG may be included in an amount of about 5 wt % to about 35 wt % of the composition. In some further embodiments, the solvent system comprising NMP may further comprise one or more PEGs having a number average molecular weight of about 400 Da or less at about 5 wt % to about 15 wt % of the composition.

The biocompatible polymer-solvent system includes a biodegradable polymer and a solvent system comprising at least one solvent and at least one component that modifies the melting point of the polymer-solvent system. The solvent system may further comprise at least one component that modifies the melting point of the biocompatible polymer-solvent system such that viscosity of the pharmaceutical composition is rendered temperature sensitive. In some embodiments, a single component of the solvent system that modifies the melting point of the biocompatible polymer-solvent system may be added to the biocompatible polymer-solvent system. In other embodiments, two components of the solvent system that modify the melting point of the biocompatible polymer-solvent system may be added to the biocompatible polymer-solvent system. In yet other embodiments, three or more components of the solvent system that modify the melting point of the biocompatible polymer-solvent system may be added to the biocompatible polymer-solvent system.

In some embodiments, the at least one component of the solvent system may be a low molecular weight PEG that modifies the melting point of the polymer-solvent system having a number average molecular weight of at least 500. In some embodiments, the at least one low molecular weight PEG may be PEG 500 or greater, wherein the PEG increases in number average molecular weight by an integer number value of 44 g/mol, which represents a single ethylene glycol (EG) monomer. As is known in the art, reference to a PEG co-solvent of a particular number average molecular weight, generally refers to a material that is not mono-disperse; i.e., there is a distribution of PEG moieties within the material that together, provide an average molecular weight of the target molecular weight. For example, PEG 500 is a mixture of PEG moieties with a molecular weight distribution that results in a number average molecular weight of 500 Da (500 Da is the target molecular weight). Accordingly, PEG 500 means PEG with a number average molecular weight of 500 Da; PEG 600 means PEG with a number average molecular weight of 600 Da, and so on. In some embodiments, suitable low molecular weight PEGs useful in the present disclosure may include, but are not limited to, PEG 500, PEG 600, PEG 1000, PEG 1450, PEG 3350, and combinations thereof. In some embodiments, a single low molecular weight PEG may be added to the biocompatible polymer-solvent system. In some embodiments, two low molecular weight PEG may be added to the biocompatible polymer-solvent system. In other embodiments, three or more low molecular weight PEGs may be added to the biocompatible polymer-solvent system.

The one or more low molecular weight PEG(s) component(s) of the solvent system that modify the melting point of the polymer-solvent system may be provided in any amount from about 1 wt % to about 95 wt %, from about 10 wt % to about 90 wt %, from about 20 wt % to about 80 wt %, from about 30 wt % to about 70 wt %, from about 40 wt % to about 60 wt %, or from about 45% to about 55% of the formulation. Alternatively, the concentration of the one or more low molecular weight PEG(s) that modify the melting point of the polymer-solvent system may range from any whole number percentage by weight percent of the formulation to any other whole number percentage by weight percent of the formulation from about 1 wt % to about 95 wt %. Similarly, the one or more low molecular weight PEG(s) component(s) of the solvent system that do not modify the melting point of the polymer-solvent system may be provided in any amount from about 1 wt % to about 95 wt %, from about 10 wt % to about 90 wt %, from about 20 wt % to about 80 wt %, from about 30 wt % to about 70 wt %, from about 40 wt % to about 60 wt %, or from about 45% to about 55% of the solvent system. Alternatively, the concentration of the one or more low molecular weight PEG(s) that do not modify the melting point of the polymer-solvent system may range from any whole number percentage by weight percent of the solvent system to any other whole number percentage by weight percent of the solvent system from about 1 wt % to about 95 wt %.

In some embodiments where the biocompatible polymer-solvent system comprises PEG 600 as the at least one component of the solvent system that modifies the melting point of the biocompatible polymer-solvent system, PEG 600 may be provided in any amount from about 15 wt % to about 70 wt % of the formulation. In some embodiments, where the biocompatible polymer-solvent system comprises PEG 1000 as the at least one component of the solvent system that modifies the melting point of the biocompatible polymer-solvent system, PEG 1000 may be provided in any amount from about 4 wt % to about 45 wt % of the formulation. In some embodiments where the biocompatible polymer-solvent system comprises PEG 1450 as the at least one component of the solvent system that modifies the melting point of the biocompatible polymer-solvent system, PEG 1450 may be provided in any amount from about 2 wt % to about 35 wt % of the formulation. In some embodiments where the biocompatible polymer-solvent system comprises PEG 3350 as the at least one component of the solvent system that modifies the melting point of the biocompatible polymer-solvent system, PEG 3350 may be provided in any amount from about 1 wt % to about 10 wt % of the formulation.

### Biocompatible Polymers

In some embodiments of the present disclosure, examples of suitable biodegradable polymers which may be used include, but are not limited to, polylactic acid, polyglycolic acid, polylactide (D,L-lactide, D-lactide, L-lactide), polyglycolide, polycaprolactones, polyanhydrides, polyamides, polyurethanes, polyesteramides, polyorthoesters, polydioxanones, polyacetals, polyketals, polycarbonates, polyphosphazenes, polyhydroxybutyrates, polyhydroxyvalerates, polyalkylene oxalates, polyalkylene succinates, poly(malic acid), polyglutamic acids, poly(alkyl cyanoacrylates) polyethylene glycol, hyaluronic acid, alginate, collagen, chitin and chitosan, and copolymers, terpolymers, and combinations or mixtures of the above materials.

In some embodiments, the biodegradable polymer may include, but is not limited to, a polylactide, a polyglycolide, a polycaprolactone, a poly(trimethylene carbonate), a polydioxanone, a copolymer thereof, a terpolymer thereof, or any combination thereof. Examples of useful materials include, but are not limited to, those polymers, copolymers or terpolymers made with lactide, lactic acid, glycolide, glycolic acid, caprolactone, p-dioxanone, trimethylene carbonate, 1,5-dioxepan-2-one, 1,4-dioxepan-2-one, ethylene oxide, propylene oxide, sebacic anhydride, and diketene acetals/diols with lower molecular weights and amorphous regions to limit crystallinity and subsequent solidification.

In some embodiments, the biodegradable polymer may be a copolymer of two monomers having a molar ratio of any two whole numbers X to Y, such that the sum of X and Y is 100. In some embodiments, the biodegradable polymer may be a copolymer of two monomers having a molar ratio of any two whole numbers X to Y, where X and Y are each at least about 10 and no more than about 90, such that the sum of X and Y is 100, e.g., a copolymer comprising a 10:90 to 90:10 molar ratio of X:Y. In some embodiments, X and Y may be at least about 15 to no more than about 85 such that the sum of X and Y is 100, e.g., a copolymer comprising a 15:85 to 85:15 molar ratio of X:Y. In some embodiments, X and Y may be at least about 25 to no more than about 75 such that the sum of X and Y is 100, e.g., a copolymer comprising a 25:75 to 75:25 molar ratio of X:Y. In some embodiments, both X and Y may be about 50, e.g., a copolymer comprising a 50:50 molar ratio of X:Y.

In some embodiments, the biodegradable polymer may be a solid polymer. The solid polymer may be a thermoplastic polymer or copolymer. Non-limiting examples of suitable solid polymers according to the present disclosure include a polymer having poly(lactide-co-glycolide) (PLG) moieties, poly(lactic acid-co-glycolic acid) (PLGA) moieties, polyethylene glycol (PEG) moieties and combinations thereof. In some embodiments, the polymer is a PLG copolymer comprising a lactide to glycolide monomer molar ratios from about 45:55 to about 99:1. In some instances, the PLG copolymer may comprise a lactide to glycolide monomer molar ratio from about 50:50 to about 90:10. In some embodiments, the polymer is a PLGA copolymer comprising a lactic acid to glycolide monomer molar ratios from about 45:55 to about 99:1. In some instances, the PLGA copolymer may comprise a lactic acid to glycolide monomer molar ratio from about 50:50 to about 90:10. In other embodiments, the polymer is a PLG-PEG and/or PLGA-PEG block copolymer where the PEG moiety has a molecular weight of about 1,000 Daltons to about 10,000 Daltons, in some embodiments about 5,000 Daltons. The PEG portion of the block copolymer ranges from about 1 wt % to about 20 wt % of the total weight of the block copolymer.

In some embodiments, the biodegradable polymer may be a liquid polymer. In some embodiments, the liquid polymer may be a copolymer comprising a first monomer comprising of lactide (D,L-lactide, D-lactide, and/or L-lactide), glycolide, or combinations thereof and a second monomer comprising caprolactone, trimethylene carbonate (TMC), or combinations thereof. Non-limiting examples of suitable liquid polymers according to the present disclosure include copolymers of D,L-lactide, D-lactide, L-lactide or glycolide, E-caprolactone, and/or trimethylene carbonate (TMC). For instance, as a specific non-limiting example, a copolymer comprising poly(D,L-lactide-co-ε-caprolactone) (PDLCL) moieties may be useful according to the present disclosure. In some embodiments, the polymer is a copolymer comprising a molar ratio of lactide (or glycolide) to caprolactone ranging from about 90:10 to about 10:90. In some embodiments, the polymer is a copolymer comprising D,L-lactide, D-lactide, L-lactide or glycolide and TMC. In some embodiments, the polymer is a copolymer comprising a molar ratio of lactide (or glycolide) to TMC ranging from about 90:10 to about 10:90. In some embodiments, the liquid copolymer may be a block copolymer comprising a first block comprising a first monomer selected from lactide (D,L-lactide, D-lactide, and/or L-lactide), glycolide, and combinations thereof and a second monomer selected from caprolactone, TMC, and combinations thereof, and a second block comprising polyethylene glycol (PEG). Alternatively, in yet other embodiments, the liquid copolymer may be a block copolymer comprising a first block comprising polyethylene glycol (PEG) and a second block comprising a first monomer selected from lactide (D,L-lactide, D-lactide, and/or L-lactide), glycolide, and combinations thereof and a second monomer selected from caprolactone, TMC, and combinations thereof.

Further non-limiting examples of suitable liquid polymers of the present disclosure include biodegradable liquid polymers comprising a copolymer with lactide (including D,L-lactide, D-lactide, and/or L-lactide) and/or glycolide residues, wherein the molar percentage of the lactide and/or glycolide residues make up greater than about 5% and less than about 95% of the polymer. In some embodiments, the lactide and/or glycolide monomer residues are present at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of total monomer residues of the copolymer. Other non-limiting examples of suitable liquid polymers of the present disclosure include biodegradable liquid polymers comprising a copolymer with caprolactone and/or TMC residues, wherein the caprolactone and/or trimethylene carbonate residues make up an amount greater than about 5% and less than about 95% of the polymer. In some embodiments, the caprolactone and/or TMC monomer residues are present at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of total monomers of the copolymer. As a further non-limiting example, a liquid polymer suitable for use with embodiments provided by the present disclosure may include a biodegradable liquid polymer having a molar ratio of about 75:25 D,L-lactide:ε-caprolactone. In yet another non-limiting example, a liquid polymer suitable according to the present disclosure may include a biodegradable liquid polymer having a molar ratio of about 75:25 D,L-lactide:trimethylene carbonate.

In some embodiments, the lactide or glycolide monomers and the caprolactone and/or TMC monomers are present in a molar ratio from about 5:95 to about 95:5, from about 10:90 to about 90:10, from about 20:80 to 80:20, from about 25:75 to about 75:25, or from about 30:70 to about 70:30. By way of non-limiting example, a first monomer of the two or more monomers may be selected from the group consisting of lactide, glycolide, and combinations thereof and a second monomer of the two or more monomers may be selected from the group consisting of caprolactone, TMC, and combinations thereof.

In some embodiments, biodegradable liquid polymers of the present disclosure may comprise a polymer block further comprising a low-molecular weight polyethylene glycol (PEG). Furthermore, the polymers may have a ratio of ethylene glycol monomer units to monomer units other than ethylene glycol (e.g., lactide, glycolide, caprolactone, TMC, or combinations thereof) ranging from about 5:95 to about 35:65, from about 10:90 to about 30:70, or from about 15:85 to about 25:75. The ratio of ethylene glycol monomer units to monomer units other than ethylene glycol may range from any whole number ratio to any other whole number ratio within the range from about 1:99 to about 40:60. In some embodiments, the ratio of ethylene glycol monomer units to monomer units other than ethylene glycol may be about 10:90, about 20:80, or about 30:70.

Biodegradable polymers of the present disclosure may comprise a block copolymer comprising at least two polymer blocks A and B. The blocks may be arranged in any number or order (e.g., as a di-block copolymer A-B, or a tri-block copolymer A-B-A or B-A-B). Such polymers are formed by initiation of the first and second monomer residues with a low-molecular weight PEG initiator. The PEG block may, in some embodiments, comprise methoxy-PEG. In block copolymers comprising a low-molecular weight PEG block, a molar ratio of ethylene glycol monomers to all other monomers with the block copolymer may be at least about 5:95, at least about 10:90, at least about 20:80, at least about 30:70, at least about 40:60, at least about 50:50, or any whole number ratio within the range from about 1:99 to about 60:40. In some embodiments, a molar ratio of ethylene glycol monomers to all other monomers with the block copolymer may be from about 10:90 to about 50:50. By way of non-limiting example, a molar ratio of a first monomer (e.g., lactide and/or glycolide) to a second monomer (e.g., caprolactone and/or TMC) to ethylene glycol in the polymer may be expressed as a whole as X:Y:Z, wherein X represents the first monomer and may be any number from about 25 to about 75; Y represents the second monomer and may be any number from about 5 to about 45; and Z represents ethylene glycol and may be any number from about 5 to about 55, such that the sum of X, Y, and Z is 100.

In some embodiments, the biodegradable polymer may be formed using an initiator selected to provide a desired structure or functionality to the polymer in the form of a particular polymer block structure or end group structure which is introduced and/or incorporated into/onto the biodegradable polymer. By way of non-limiting example, the polymer may be formed using a low-molecular weight PEG as an initiator, which may result in the formation of a block copolymer comprising a low-molecular weight PEG block. By way of another non-limiting example, the polymer may be formed using an organic acid (e.g., a hydroxy acid such as, for instance, glycolic acid) as the initiator which may result in the formation of a polymer comprising at least one carboxylic acid end group. By way of another non-limiting example, the polymer may be formed using a monofunctional alcohol (e.g., dodecanal) as the initiator which may result in the formation of a polymer comprising at least one hydroxy end group. By way of a further non-limiting example, the polymer may be formed using a diol (e.g., hexanediol) as the initiator which may result in the formation of a polymer comprising at least one hydroxy end group and that is also substantially free of terminal carboxy end groups.

In some embodiments, biodegradable polymers suitable for use in formulations according to the present disclosure may, generally, comprise a weight average molecular weight ranging from about 1 kDa and about 100 kDa. In some embodiments, the biodegradable polymer may comprise a weight average molecular weight ranging from about 1 kDa to about 5 kDa, from about 1 kDa to about 10 kDa, from about 1 kDa to about 15 kDa, from about 1 kDa to about 20 kDa, from about 1 kDa to about 25 kDa, from about 1 kDa to about 30 kDa, from about 1 kDa to about 40 kDa, from about 1 kDa to about 50 kDa, from about 1 kDa to about 60 kDa, from about 1 kDa to about 70 kDa, from about 1 kDa to about 80 kDa, from about 1 kDa to about 90 kDa, from about 1 kDa to about 100 kDa, or any value to any other value, in whole number increments, from about 1 kDa to about 100 kDa. In some embodiments, the polymer has a weight average molecular weight of at least about 1 kDa, at least about 5 kDa, at least 10 kDa at least about 15 kDa, at least 20 kDa, at least about 25 kDa, at least about 30 kDa, at least about 35 kDa, at least 40 kDa, at least about 45 kDa, at least about 50 kDa, at least about 55 kDa, at least about 60 kDa, at least about 70 kDa, at least about 75 kDa, at least about 80 kDa, at least about 85 kDa, at least about 90 kDa, at least about 95 kDa, or at least about 100 kDa.

In some embodiments of the composition, the biodegradable polymer may make up about 0.1 wt % to about 50 wt %, about 5 wt % to about 45 wt %, about 10 wt % to about 40 wt %, about 15 wt % to about 35 wt %, or about 20 wt % to about 30 wt % of the composition. In some embodiments, the biodegradable polymer may make up about 20 wt %, about 25 wt %, or about 30 wt % of the composition. Alternatively, the biodegradable polymer may make up any whole-number weight percentage of the composition from about 1 wt % to about 50 wt %. In other embodiments, the biodegradable polymer may make up any tenth of a whole number percent of the composition from about 0.1 wt % to about 50 wt %.

### Active Pharmaceutical Ingredient (API)

APIs (also referred to herein as drugs or active pharmaceutical ingredients or agents) which may be suitable for the present disclosure include biologically active agents that provide a therapeutically useful biological effect. APIs may act locally and/or systemically in the treatment, therapy, cure, and/or prevention of a disease, disorder, ailment, or may otherwise provide a health or medical benefit to a subject. In some embodiments, examples of such drugs include, without limitation, antimicrobials, anti-infectives, anti-parasitic drugs such as avermectins, anti-allergenics, steroidal anti-inflammatory agents, non-steroidal anti-inflammatory agents, anti-tumor agents, anticancer drugs, decongestants, miotics, anti-cholinergics, sympathomimetics, sedatives, hypnotics, psychic energizers, tranquilizers, endocrine/metabolic agents, hormones (e.g., androgen, anti-estrogen, estrogen, gonadotropin-releasing hormone analogues, testosterone and progesterone), drugs for the treatment of diabetes, drugs for the treatment of dementia (e.g., Alzheimer's disease), GLP-1 agonists, androgenic steroids, estrogens, progestational agents, LHRH agonists and antagonists, somatotropins, narcotic antagonists, prostaglandins, analgesics, antispasmodics, antimalarials, antihistamines, cardioactive agents, antiparkinsonian agents, antihypertensive agents, anti-virals, antipsychotics, immunosuppressants, anesthetics, antifungals, anti proliferatives, anticoagulants, antipyretics, antispasmodics, anti-aging agents, and nutritional agents. APIs of the foregoing classes and specific APIs described herein may be administered in various forms, including as base form, salts, esters, complexes, prodrugs and analogs of the foregoing.

In some embodiments, APIs useful in embodiments provided by the present disclosure may include peptide drugs, such as a peptide or a polypeptide. Examples of peptides and polymeric drugs which may be suitable for the present application include, but are not limited to, degarelix, abaloparatide, teriparatide, leuprolide (leuprorelin), exenatide, liraglutide, albiglutide, dulaglutide, basal insulin, octreotide, goserelin, triptorelin, nafarelin, buserelin, histrelin, deslorelin, ganirelix, abarelix, cetrorelix, teverelix, lanreotide, carfilzomib, human growth hormone, interferon-alpha, interferon-beta, interferon-gamma, interleukin, calcitonin, growth hormone releasing peptides, glucagon-like peptides, granulocyte-colony stimulating factor, nerve growth factor, platelet-derived growth factor, insulin-like growth factor, vascular endothelial growth factor, fibroblast growth factor, octreotide, bone morphogenic protein, erythropoietin, albiglutide, anaritide, angiotensin II, buforin II, calcitonin, carperitide, cecropin P1, cetrorelix, dermaseptin, desmopressin, drosocin, enfuvirtide, etelcalcetide, exenatide, indolicidin, liraglutide, lixisenatide, magainin I, magainin II, nesiritide, neurotensin, pramlintide, ranalexin, semaglutide, tachyplesin, teduglutide, vasopressin, 1-deamino-8-D-arginine vasopressin, and salts, complexes, prodrugs, and analogs thereof. As a non-limiting example of salts of a drug such as leuprolide, which may be useful to embodiments provided by the present disclosure include, but are not limited to, leuprolide acetate, leuprolide hydrochloride, leuprolide mesylate, and leuprolide trifluoroacetate.

APIs useful in the present disclosure may also include, but are not limited to, a small molecule organic compound. The small molecule drug may be a hydrophobic drug, such as corticosteroids such as prednisone, prednisolone, beclomethasone, fluticasone, methylprednisone, triamcinolone, clobetasol, halobetasol, and dexamethasone; azole medications such as metronidazole, fluconazole, ketoconazole, itraconazole, miconazole, dimetridazole, secnidazole, ornidazole, tinidazole, carnidazole, and panidazole; sex steroids such as testosterone, estrogens such as estradiol, and progestins, including esters thereof; statin drugs such as atorvastatin, simvastatin, fluvastatin, lovastatin, pitavastatin, pravastatin, and rosuvastatin; and antiandrogen drugs such as abiraterone, galeterone, orteronel, and enzalutamide, and salts, esters, complexes, prodrugs and analogs of the foregoing. As a non-limiting example, esters of a drug such as testosterone, which may be useful to the present disclosure, include, but are not limited to, testosterone undecanoate (TU, also known as testosterone undecylate), testosterone cypionate (TC), testosterone propionate, testosterone enanthate (TE), and testosterone busiclate.

Examples of specific additional drugs that may be utilized include, but are not limited to, hydrophilic and hydrophobic small molecule drugs such as rivastigmine tartrate, cisplatin, carboplatin, paclitaxel, rapamycin, tacrolimus (fujimycin), bortezomib, trametinib, methotrexate, riociguat, macitentan, sumatriptan, anastozole, fulvestrant, exemestane, misoprostol, follicle-stimulating hormone, axitinib, paricalcitol, pomalidomide, dustasteride, doxycycline, doxorubicin, ciprofloxacin, quinolone, ivermectin, eprinomectin, doramectin, leflunomide, teriflunomide, haloperidol, diazepam, risperidone, olanzapine, amisulpride, aripiprazole, asenapine, clopazine, iloperidone, lurasidone, paliperidone, quetiapine, ziprasidone, bupivacaine, lidocaine, ropivacaine, naltrexone, fentanyl, buprenorphine, butorphanol, loperamide, fingolimod, apomorphine, brexpiprazole, levothyroxine and salts and esters thereof, as well as, complexes, prodrugs, and analogs thereof. In some embodiments of the present disclosure, the API may be a prodrug, such as, by way of non-limiting example. Where the API is a prodrug, the drug may be, inter alia, a hydrophobic or hydrophillic salt or a covalently bound ester of the corresponding drug, or covalently bound to the polymer itself. Providing a prodrug as the API may provide advantages or benefits in certain applications. By way of non-limiting example, providing the API as a prodrug may improve the stability of the formulation (e.g., during irradiation, while in storage, or after delivery in vivo), delay the release of the active form of the drug, affect or modify the solubility of the drug in the formulation, and/or extend or otherwise modify the duration of action of the drug. Where the prodrug is a covalently bound ester of the corresponding drug, the ester is often hydrolyzed in vivo to the corresponding carboxylic acid, which is then removed to convert the drug to its active form. This mechanism may be particularly beneficial where a low burst release and/or low peak plasma concentration of the drug is desirable. In some embodiments, a desired release profile may be obtained by providing the API as a mixture of a prodrug and the corresponding drug in a predetermined ratio.

In some embodiments of the present disclosure, the API may be provided in crystalline form. In these embodiments, a selection of API crystal shape, or habit, may be another consideration in the preparation of the polymer formulation, as different crystal habits may result in different release profiles. The selection of crystal habit will largely depend upon the API and desired release profile, but in general, the crystal habit should be stable throughout all manufacturing, shipping, and delivery conditions, e.g., during polymer formulation preparation, e-beam irradiation, shipping and storage, mixing, injection, etc. Additionally, different crystal habits may be more or less likely to form hydrates or polymorphs, which may be desirable or undesirable depending upon the application, but it is generally advantageous that the transition into the hydrate or polymorph be predictable and/or controllable. Selection of a crystal habit can be based on these and other considerations. In some embodiments, the API is in a crystalline form having a block-like crystal habit or a needle-like crystal habit.

In some embodiments, the desired particle size, or distribution of particle sizes, of the API will largely depend upon the API and the desired release profile. In general, a smaller particle size will result in more rapid release of the API in vivo (i.e., shorter duration of release) and/or a larger burst and corresponding higher peak concentration in vivo. Meanwhile, in general, a larger particle size will result in slower release of the API in vivo (i.e., longer duration of release) and/or a smaller burst and corresponding lower peak concentration in vivo. Where the polymer formulation is an injectable formulation, the gauge of the needle used to inject the formulation may also be a consideration in selecting a particle size, because large API particles may clog a large-gauge (i.e., small-diameter) needle or result in excessive injection force. In some embodiments, a unimodal particle size distribution may provide an advantageous release profile or other desirable effect. In some other embodiments, a bimodal particle size distribution may provide an advantageous release profile or other desirable effect. By way of non-limiting example, and without wishing to be bound by any particular theory, it may be possible that smaller particles may cause rapid drug release (e.g., by faster release from a depot and/or faster solubilization upon release and/or modification of fluid channels in the depot) to provide an initial therapeutic effect, and larger particles may be released later to provide an extended therapeutic effect. In some instances, embodiments may also comprise particles of the API that have been encapsulated in, e.g., a microsphere or lipid sphere, which may provide an additional mechanism for controlling release of the API in vivo.

As used herein, unless otherwise specified, the term “particle size” refers to a median particle size determined by volume-based particle size measurements, such as, by way of non-limiting example, by use of a laser diffraction particle size analyzer such as a Malvern Mastersizer® instrument. Such particle sizes may also be referred to as “D_{v,50}” values. Furthermore, as used herein, unless otherwise specified, the term “span” refers to the difference between a 90th percentile particle size (referred to as “D_{v,90}”) and a 10th percentile particle size (referred to as “D_{v,10}”), divided by the 50th percentile particle size. Thus, the span of a volume of particles can be interpreted as a measure of how broadly distributed particle sizes are within the volume. In various embodiments, the API will have a median particle size (D_{v,50}) of from about 10 μm to about 200 μm, from about 10 μm to about 180 μm, from about 10 μm to about 160 μm, from about 10 μm to about 140 μm, from about 10 μm to about 120 μm, from about 10 μm to about 100 μm, from about 15 μm to about 100 μm, from about 15 μm to about 90 μm, from about 15 μm to about 80 μm, from about 20 μm to about 70 μm, from about 20 μm to about 60 μm, from about 25 μm to about 50 μm, from about 30 μm to about 90 μm, from about 40 μm to about 90 μm, from about 50 μm to about 90 μm, from about 60 μm to about 90 μm, or from about 70 μm to about 90 μm. In other embodiments, the median particle size of the API in compositions of the present disclosure may range from any whole number to any other whole number within the range of from about 1 μm to about 250 μm. Additionally, in various embodiments, the API may have a particle size span of from about 0.1 to about 8, from about 0.5 to about 8, from about 1 to about 8, from about 1.5 to about 8, from about 2 to about 7, from about 3 to about 6, or from about 4 to about 5. In other embodiments, the API may have a particle size span from about 1.5 to about 5, from about 1.5 to about 6, from about 2 to about 6, from about 2 to about 5, or from about 2 to about 4. Similarly, in yet other embodiments, the API may have a particle size span of about 1, about 1.5, about 2, about 2.5, about 3, about 4, about 4.5, about 5, about 5.5, about 6, about 6.5, about 7, about 7.5, about 8, or alternatively about any tenth of a whole number from about 1 to about 8.

The concentration of the API in compositions of the present disclosure depends on the drug that is included in the composition and may range from 0.1% to 50% by weight of the composition, including any whole number percent to any other whole number percent within the range of from about 1 percent to about 50 percent by weight. In other embodiments, the amount of API in compositions of the present disclosure may range from any tenth of a percent to any other tenth of a percent within the range of from about 0.1 percent to about 50 percent by weight. In some embodiments of the present disclosure, the concentration of the API is no more than about 25% by weight.

As noted above, in some embodiments the API may be substantially in solid form (i.e., solid particles of the API are suspended in the solid or liquid polymer-solvent composition), while in other embodiments the API may be dispersed in the polymer-solvent composition. As used herein, unless otherwise noted, use of the term “suspension” when referring to a composition of the present disclosure may refer to formulations in which at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the API is in the form of solid particles suspended in the polymer-solvent composition. Description of an API herein as being “substantially in solid form” or “substantially in suspension” in a formulation refers to formulations in which at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the API is in the form of solid particles suspended in the polymer-solvent system.

### Administration

Pharmaceutical compositions according to the present disclosure may be provided as a part of a delivery system comprising a syringe, wherein the pharmaceutical composition or formulation is contained within the syringe. Accordingly, such delivery systems are likewise within the scope of the present disclosure. In some embodiments, the syringe may comprise a 6 to 32 or larger gauge needle. In other embodiments, the syringe may comprise an 18 to 30 gauge needle. In some embodiments, the syringe may be an auto-injector syringe. In some embodiments, the pharmaceutical compositions according to the present disclosure may be stored at refrigeration or cold storage temperatures from about 0° C. to about 8° C. and then warmed room temperature from about 18° C. to about 25° C. prior to administration to a subject. In some embodiments, the pharmaceutical compositions may not need to be re-mixed or may be subjected to minimal re-mixing, agitation, shaking, or otherwise disturbing to restore the dosage uniformity prior to being administered to a patient. Upon injection of the pharmaceutical composition into the body and contact of the composition with a bodily fluid, the solvent dissipates and an in situ liquid or solid implant forms.

The in-situ liquid or solid implant may release the API over an extended time period. In various embodiments, an API within a pharmaceutical composition according to the present disclosure is released into a patient, for example (as determined by measuring blood serum levels of the API in a patient) for greater than about three days, greater than about one week, greater than about two weeks, greater than about three weeks, greater than about four weeks, greater than about eight weeks, greater than about twelve weeks, greater than about sixteen weeks, greater than about 20 weeks, greater than about 24 weeks, greater than about 32 weeks, greater than about 48 weeks, greater than about 72 weeks, greater than about 96 weeks, or greater than about 144 weeks. In various embodiments, an API within a pharmaceutical composition according to the present disclosure is released into a patient, for example (as determined by measuring blood serum levels of the API in a patient) for greater than about 2 weeks, greater than about 1 month, greater than about 2 months, greater than about 3 months, greater than about 4 months, greater than about 5 months, greater than about 6 months, greater than about 9 months, greater than about 12 months, greater than about 18 months, greater than about 24 months, or greater than about 36 months. Such levels of API may be at levels having a pharmacologic or therapeutic effect.

### EXAMPLES

### Example 1: Testosterone Undecanoate Inhomogeneity within a Poly(D,L-Caprolactone-Co-Lactide) Liquid Polymer Formulation Cannot be Restored by Mixing

A known property of suspensions and/or dispersions, i.e., any mixture comprising more than a single phase, is that the phases will inherently separate over time or that they can be forced to separate by applying an external force. Thus, following storage in a single-syringe at a given temperature for an extended period of time, the suspended API may undergo separation from the polymer-solvent system.

Separation of API particles from a liquid polymer-solvent system was observed to occur for syringes containing a testosterone undecanoate (TU)-liquid polymer formulation comprising, by weight, 20% TU, 30% of a 14 kDa 75:25 poly(D, L-lactide-co-c-caprolactone) (PDLCL)-acid-initiated polymer, 25% N-Methyl-2-Pyrrolidone (NMP), and 25% PEG 300, following storage at room temperature for 6 months. Separation of API particles from a liquid polymer-solvent system could be replicated by centrifuging syringes containing the same formulation at 2,000 rpm for 25 minutes at 20° C. These conditions, referred to as “centrifuged simulated long-term storage”, were able to simulate the TU particle-polymer phase separation observed to naturally occur after long-term storage of the formulation. FIG. 1 shows a representative syringe following centrifuged induced separation with the syringe tip facing outward away from the axis of rotation during centrifugation.

To restore formulation homogeneity following TU particle separation from the liquid polymer-solvent system upon centrifuged simulated long-term storage, mixing studies were conducted. Centrifuged syringes containing the aforementioned TU-liquid polymer formulations were coupled to an empty Schott mixing syringe and then mixed back and forth for 0, 0.5, 5, 10, 15, or 25 mixing cycles. Each mixing cycle comprises a full depression and retraction of the syringe plunger rod. For each mixing cycle, samples were prepared in triplicate.

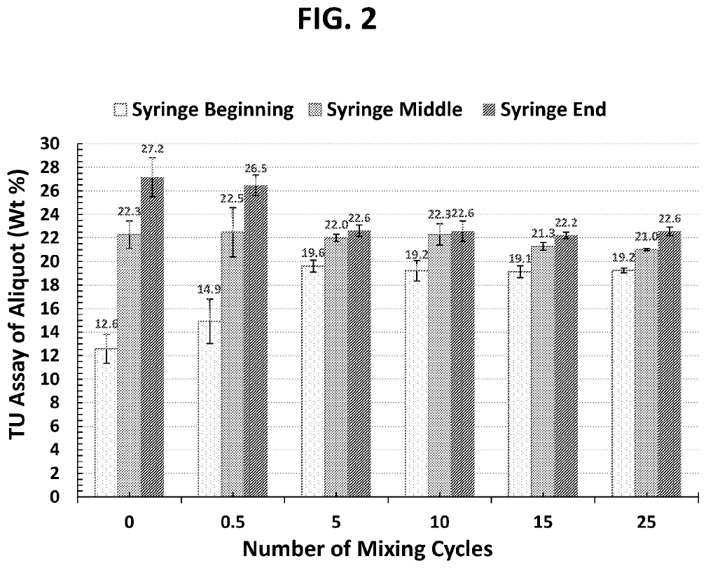

Sample aliquots were withdrawn from the beginning, middle, and end of the syringe (relative to the syringe tip) and delivered into a tared 100 mL volumetric flask to which 50 mL of acetonitrile was subsequently added. These volumetric flasks were then mixed to fully dissolve the formulation sample aliquot. Dissolved samples were then subjected to HPLC for TU content analysis. FIG. 2 shows the amount of TU collected from within each region of the mixing syringe after a certain number of mixing cycles. Following centrifuged simulated long-term storage, TU particles separated away from the denser liquid polymer phase. Before any re-mixing, the TU content within the beginning, middle, and end regions of the syringe varied. While dose uniformity could be partially improved with re-mixing, dose inhomogeneity remained after 5 mixing cycles. Even after 25 mixing cycles, restoration of the original dose uniformity that was present prior to centrifuged simulated long-term storage could not be achieved to a desirable extent.

### Example 2: Preparation of a Temperature Sensitive High-Viscosity Poly(D,L-Caprolactone-Co-Lactide) Liquid Polymer Formulation Possessing Suitable Melt Characteristics

The preparation of a non-flowable, high viscosity formulation at low temperatures (i.e., ≤8° C.) that can convert to a flowable, low viscosity formulation upon warming to higher temperatures (i.e., ≥18° C.) for clinical administration, such that the API homogeneity is maintained within the syringe in order to ensure that a uniform API dose is delivered when administered without needing to re-mix, or with minimal re-mixing, the suspension was investigated.

To establish liquid polymer formulations with a temperature sensitive viscosity, liquid polymer formulations comprising, by weight, 37.5% of a 5 kDa 75:25 PDLCL glycolic acid-initiated liquid polymer were prepared with the addition of either NMP, PEG 300, PEG 400, PEG 600, or combinations thereof. Of these various formulations, those with PEG 600 displayed a change in viscosity as evidenced by visual solidification when the formulations were cooled from room temperature to 5° C. For those formulations that solidified at low temperature, they appeared uniphasic (i.e., as a solid mass) despite only the PEG 400 and/or PEG 600 parts of the solvent system being known to freeze at temperatures of <5° C. Furthermore, these solidified samples showed no signs of liquid phase migration when stored vertically for days. Throughout the Examples, the formulations that solidify upon cooling to a temperature from about 0° C. to about 8° C. are referred to as “solidifying” formulations, compositions, or samples; whereas the formulations that did not solidify upon cooling are referred to as “non-solidifying” formulations, compositions, or samples.

As a visual indication that these low temperatures induced solidified formulations possessed a high viscosity capable of maintaining the homogeneity of the initial suspension, the location of air bubbles, which were frozen in place upon solidification remain unaltered when subjected to centrifugation. However, once these solidified formulations were re-warmed to room temperature, they returned to a flowable liquid state, wherein the previously frozen-in-place air bubbles were capable of moving freely within the syringe.

To characterize the freezing and melting characteristics of these various formulations, Differential Scanning calorimetry (DSC) thermal analysis was conducted. DSC scans were collected by holding the samples at an initial temperature of 20° C. for 1 minute before being cooled to −20° C. at a rate of 10° C./minute. Samples were then held at −20° C. for 5 minutes before being warmed to 45° C. at a rate of 5° C./minute. The results from the DSC thermal analysis are depicted as a plot with a shaded area under the curve (AUC) representing the calorimetric difference between the sample (i.e., the solidifying liquid polymer formulation) and the control (i.e., the pan) used within the calorimeter. This calorimetric difference is the amount of additional heat needed to melt the solidified liquid polymer formulation when compared to the control. The slopes associated with the AUC are used to calculate the physical state transitions, i.e., the Melt Onset Temperature and the Melt Peak Temperature. Table 1 summarizes the results from the DSC thermal analysis of the various formulations. As a general, non-limiting consideration, potential formulations of interest may be identified as those which are generally: 1) substantially non-flowable with high viscosity at lower temperatures which may be demonstrated, for example, by a Melt Onset Temperature of about 12° C. or lower and 2) substantially flowable with low viscosity at higher temperatures which may be demonstrated by a Melt Peak Temperature of about 22° C. or less. DSC data analysis identified multiple formulations with a Melt Onset Temperature of about 5° C. or higher, in some embodiments about 8° C. or higher and a Melt Peak Temperature of about 20° C. or less. As a non-limiting example, the DSC melt profile of formulation No. 17 is shown in FIG. 3.

TABLE 1
DSC Melt Data And Viscosities Of PDLCL Acid-Initiated 5
kDa Liquid Polymer Formulations With Different MW PEGs

| | | | | | | DSC Melt | DSC Melt |
|---|---|---|---|---|---|---|---|
| | Polymer | NMP | PEG 300 | PEG 400 | PEG 600 | Onset | Peak |
| Formulation No. | (%) | (%) | (%) | (%) | (%) | Temp (° C.) | Temp (° C.) |
| 1 | 37.5 | 31.3 | 31.3 | — | — | N/A | N/A |
| 2 | 37.5 | — | 62.5 | — | — | N/A | N/A |
| 3 | — | — | 62.5 | — | — | N/A | N/A |
| 4 | 37.5 | — | — | 62.5 | — | −1.39 | 5.55 |
| 5 | 37.5 | — | 15.6 | 46.9 | — | N/A | N/A |
| 6 | 37.5 | 15.6 | — | 46.9 | — | N/A | N/A |
| 7 | 37.5 | 15.6 | — | — | 46.9 | 4.19 | 15.67 |
| 8 | 37.5 | — | — | 15.6 | 46.9 | 9.07 | 17.94 |
| 9 | 37.5 | — | 15.6 | — | 46.9 | 6.66 | 17.97 |
| 10 | 37.5 | 15.6 | — | 31.3 | 15.6 | −4.53 | 6.97 |
| 11 | 37.5 | 15.6 | — | 15.6 | 31.3 | −1.48 | 11.62 |
| 12 | 37.2 | 7.5 | — | — | 55.3 | 11.56 | 20.86 |
| 13 | 37.6 | 7.7 | — | 8.1 | 46.6 | 7.58 | 19.58 |
| 14 | 37.7 | 7.9 | — | 15.3 | 39.0 | 3.82 | 16.72 |
| 15 | 37.4 | — | — | 31.1 | 31.5 | 4.24 | 16.01 |
| 16 | 37.3 | — | 31.5 | — | 31.2 | 0.60 | 15.30 |
| 17 | 37.5 | 9.3 | 9.3 | — | 43.9 | 3.88 | 16.05 |

N/A: Data not available.

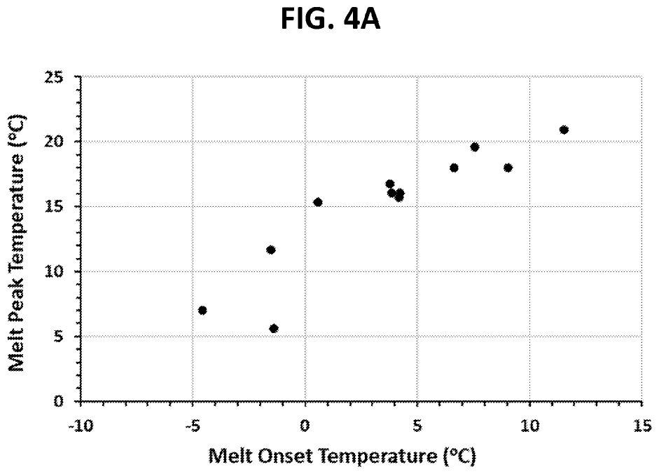

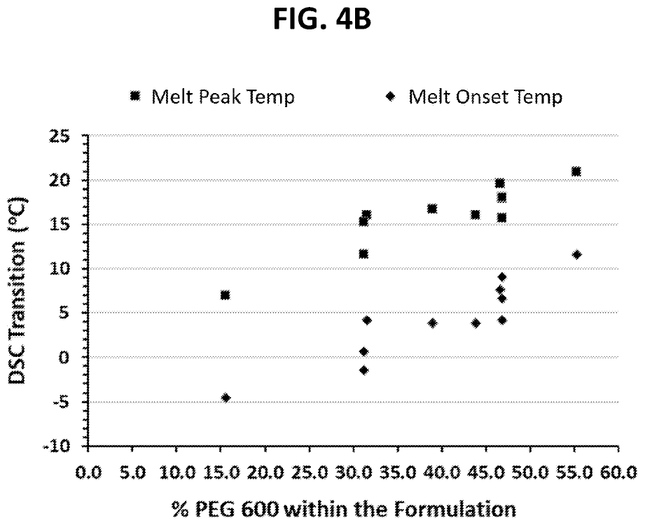

The Melt Onset Temperature versus the Melt Peak Temperature for the various solidifying formulations of Table 1 are plotted in FIG. 4A. The DSC phase transition temperatures as a function of the percentage of PEG 600 within the formulation for the solidifying formulations are plotted in FIG. 4B. The Melt Onset Temperatures are shown by the diamonds (♦) and the Melt Peak Temperatures are shown on the plots by the squares (▪). In general, as the percentage of PEG 600 in the formulation increases, both the Melt Onset Temperatures and Melt Temperatures increase.

### Example 3: Preparation of a Temperature Sensitive High-Viscosity Testosterone Undecanoate Poly(D,L-Caprolactone-Co-Lactide) Liquid Polymer Formulation that Maintains TU-Polymer Suspension Homogeneity

A TU-liquid polymer formulation comprising, by weight, 20% TU, 30% of a 5 kDa 75:25 PDLCL glycolic acid-initiated polymer, 7.5% NMP, 7.5% PEG 300, and 35% PEG 600 was prepared. This formulation was then cooled to 4° C. and subjected to centrifugation at 2,000 rpm for 60 minutes. The conditions are referred to as “prolonged centrifuged simulated storage”. The TU dose uniformity was analyzed before and after being subjected to prolonged centrifuged simulated storage. As a comparative control, the TU dose uniformity for a non-solidifying TU-liquid polymer formulation comprising, by weight, 20% TU, 30% of a 14 kDa 75:25 PDLCL acid-initiated polymer, 25% NMP, and 25% PEG 300 was also analyzed after simulated storage. All formulations were analyzed in triplicate and none of the samples were re-mixed prior to TU dose uniformity analysis.

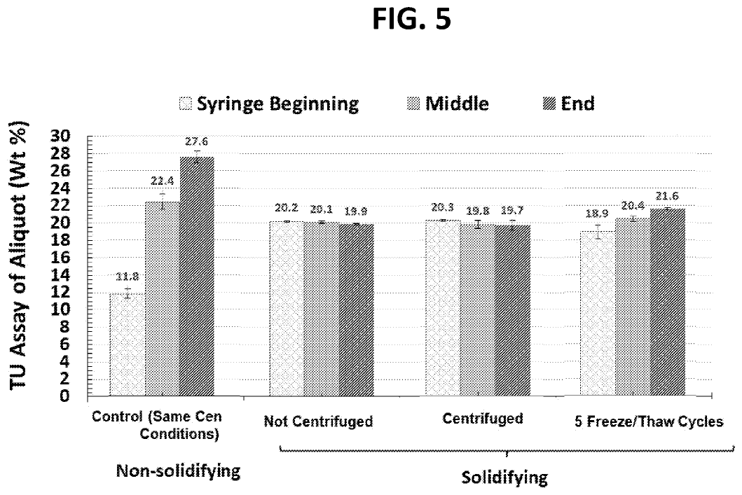

FIG. 5 shows the results of the TU dose uniformity for the analyzed samples. The dose uniformity of the non-solidifying control TU-liquid polymer formulation experiences significant TU inhomogeneity upon prolonged centrifuged simulated storage. In contrast, the solidifying TU-liquid polymer formulation comprising additional PEG 600 does not undergo any significant TU inhomogeneity following being subject to freezing and thawing. When the same solidifying TU-liquid polymer formulation is centrifuged to complete the simulated storage, the TU dose uniformity remains unchanged. TU dose uniformity of the solidifying TU-liquid polymer formulation is relatively well maintained even after 5 cycles of freezing/thawing. The slight TU dose inhomogeneity that results after 5 cycles of freezing/thawing is still significantly better than the TU dose inhomogeneity seen in the non-solidifying TU-liquid polymer formulation control after a single stimulated storage cycle.

The ability of PEG 600 to induce a low temperature sensitive high viscosity within the TU-liquid polymer formulation is useful in maintaining the TU particle distribution within the liquid polymer phase when subjected to aggressive centrifugation to stimulate prolonged storage. These characteristics make these formulations suitable for clinical use as they will be solidified at clinical storage conditions (i.e., refrigeration temperatures from about 0° C. to about 8° C.) but fully melted as a flowable formulation ready for administration by room temperature (i.e., at temperatures from about 18° C. to about 25° C.). As such, a physician merely needs to remove a syringe with the solidified formulation from cold storage and allow it to equilibrate to room temperature before administering to a patient, such that the dose uniformity is well-maintained without the need to re-mix the melted formulation.

### Example 4: Preparation of Temperature Sensitive High-Viscosity Poly (D,L-Lactide-Co-Glycolide) Solid Polymer Formulations

To establish temperature sensitive, high viscosity formulations comprising solid polymers, formulations were prepared comprising, by weight, 34% or 37.5% of a 56 kDa 50:50 poly(D,L-lactide-co-glycolide) (PLG)-acid initiated solid polymer with the addition of various amounts of either NMP, PEG 300, PEG 600, or combinations thereof, such that they total to 100%. In order to assess the amount of PEG 600 suitable for rendering the temperature-sensitive solidification properties as seen in the liquid polymer formulations in the previous examples, increasing amounts of PEG 600 was added to solid polymer formulations. However, to accommodate the increasing amount of PEG 600, the amount of NMP was correspondingly reduced, such that the amounts of all the parts, by weight percentage, totaled to 100% and the weight percentage of the polymer remained constant. As a consequence of this, the percentage of PEG 600 relative to the amount of solvents in the formulation steadily increased up to 90%.

To characterize the freezing and melting characteristics of these various formulations, DSC thermal analysis was conducted. DSC scans were conducted by holding the samples at an initial temperature of 20° C. for 1 minute before being cooled to −20° C. at a rate of 10° C./minute. Samples were then held at −20° C. for 10 minutes before being warmed to 50° C. at a rate of 5° C./minute. Suitable solidified formulations were screened for those which began to melt at about 5° C. or higher and which were fully melted as a flowable suspension by at about 20° C. or higher. Table 2 summarizes the results from the DSC thermal analysis for the various solid polymer formulations.

TABLE 2
DSC Melt Data and Viscosities of PLG acid-initiated
56 kDa Solid Polymer Formulations with PEG 600

| | | | | | DSC Melt | DSC Melt |
|---|---|---|---|---|---|---|
| | Polymer | NMP | PEG 300 | PEG 600 | Onset | Peak Temp |
| Formulation No. | (%) | (%) | (%) | (%) | Temp (° C.) | (° C.) |
| 1 | 34.0 | 66.0 | — | — | N/A | N/A |
| 2 | 34.0 | 52.8 | — | 13.2 | N/A | N/A |
| 3 | 34.0 | 39.6 | — | 26.4 | −12.76 | 1.10 |
| 4 | 34.0 | 26.4 | — | 39.6 | −4.58 | 8.92 |
| 5 | 34.0 | 19.8 | — | 46.2 | −1.27 | 11.37 |
| 6 | 34.0 | 13.2 | — | 52.8 | 4.93 | 14.95 |
| 7 | 34.0 | 6.6 | — | 59.4 | 9.57 | 17.78 |
| 8 | 34.0 | 9.9 | 9.9 | 46.2 | 4.13 | 14.57 |
| 9 | 37.5 | 9.4 | 9.4 | 43.8 | 3.80 | 14.24 |

N/A: Data not available

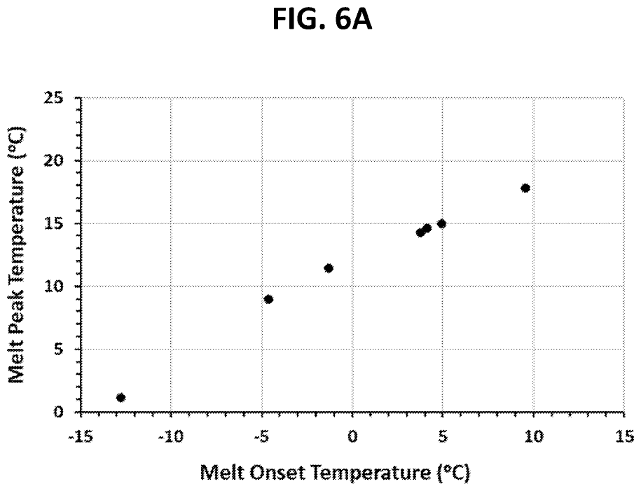

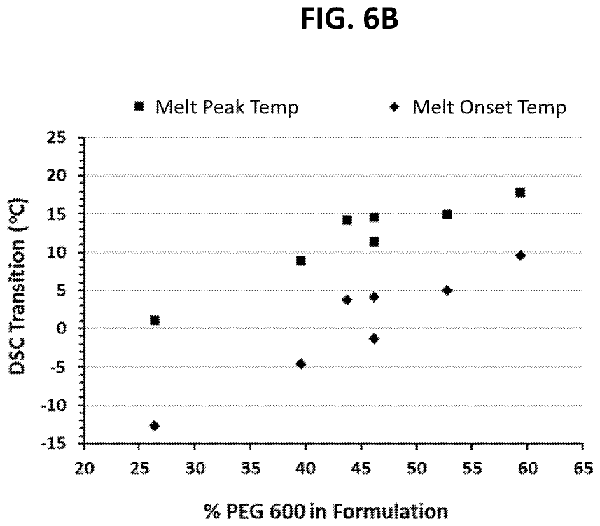

The Melt Onset Temperature versus the Melt Peak Temperature for the various solidifying formulations of Table 3 are plotted in FIG. 6A. The DSC transition temperature as a function of the percentage of PEG 600 within the formulation for the tested formulations are plotted in FIG. 6B. The Melt Onset Temperatures are shown by the diamonds (♦) and the Melt Peak Temperatures are shown on the plots by the squares (▪). Of the tested solidifying solid polymer formulations, DSC data analysis demonstrated multiple formulations with suitable solidification properties at temperatures at about 5° C. or below with suitable non-solidification properties at temperatures at about 20° C. or higher. As a non-limiting example, formulations which possess a Melt Onset Temperature of about 8° C. or higher and a Melt Peak Temperature of about 20° C. or less are of interest as these formulations are well suited for clinical use (i.e., cold storage and administration to a patient at room temperature).

Of the tested formulations, four demonstrated suitable temperature transition states. Test formulation Nos. 6, 8, and 9 possessed a Melt Onset Temperature of about 4° C. with a Melt Peak Temperature of about 14-15° C. Test formulation No. 7 possessed a Melt Onset Temperature of about 10° C. with a Melt Peak Temperature of about 18° C. However, the amount of PEG 600 that achieved suitable solidification properties in test formulation No. 7 was higher than that seen used in the liquid polymer formulations. For instance, solidifying solid polymer test formulation No. 8 contained an amount of PEG 600 at 46.2% of the formulation to achieve a similar Melt Onset and Melt Peak Temperatures as that seen solidifying liquid polymer test formulation No. 16 from Table 1 of Example 3, which contained an amount of PEG 600 at 31.5% of the formulation.

### Example 5: Preparation of Temperature Sensitive High-Viscosity Poly(D,L-Lactide-Co-Glycolide) Solid Polymer Formulations with PEG 600 Added after Formulation

In previous Example 4, PEG 600 was added to the solid polymer composition directly during its formulation. The weight percentage of the polymer in those formulations was fixed such that as the amount of PEG 600 increased and the amount of NMP correspondingly decreased. In order to lower the PEG 600 content as a percentage of the solvent, PEG 600 was added to a pre-formulated solid polymer formulation, instead of being prepared by adding it during the initial formulation.

To a first set of HSW syringes (n=6) increasing amounts of PEG 600 was initially added. Specifically, 62.2 mg, 95.1 mg, 151.3 mg, 227.6 mg, 337.6 mg, or 527.1 mg of PEG 600 was added to the syringe. These syringes were then interconnected to a second syringe containing a fixed amount of a pre-formulated solid polymer formulation comprising, by weight, 34% of a 56 kDa 50:50 PLG acid-initiated solid polymer and 66% of NMP. The contents of the two syringes were then vigorously mixed such that a homogenous suspension was achieved. In contrast to the formulations in Example 4, as PEG 600 was introduced into a pre-formulated solid polymer formulation, the percentage of PEG 600 relative to total solvent content was effectively reduced in the “pre-mixed” formulations.

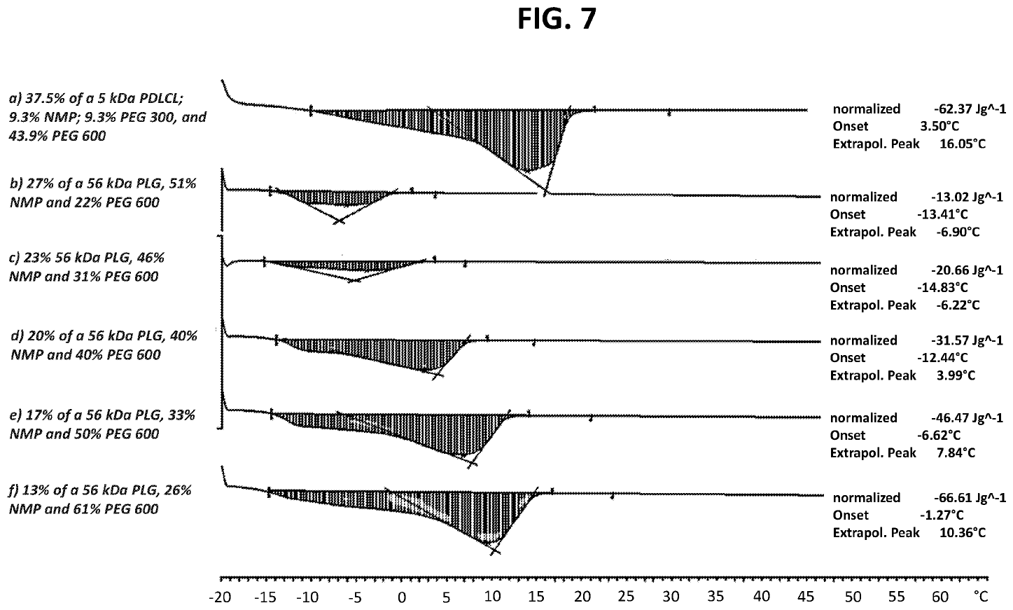

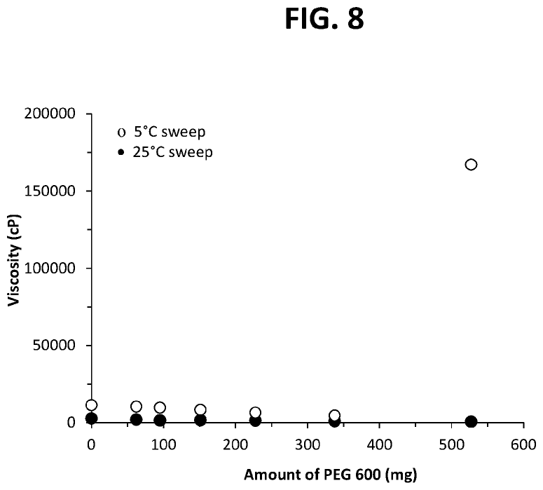

From visual observations, solid polymer formulations with PEG 600 added did not appear to be fully solidified until the amount of added PEG 600 was at about 50% of the formulation or above. These visual observations appear to be supported through DSC thermal analysis. Table 3 and FIG. 7 shows DSC data and melt profiles for the samples where PEG 600 was added directly into syringes containing pre-formulated solid polymer formulations. For reference, a liquid polymer formulation comprising, by weight, 37.5% of a 5 kDa 75:25 PDLCL glycolic acid-initiated polymer, 9.3% NMP, 9.3% PEG 300, and 43.9% PEG 600 (formulation No. 17 from Table 1 in Example 2) was also included in FIG. 7. This reference formulation demonstrated a DSC Melt Onset Temperature and Melt Peak Temperature of 3.88° C. and 16.05° C., respectively. These observations were further supported by analyzing the viscosities of the test formulations using hybrid rheometry. The measured viscosities at either 25° C. or 5° C. for these solidifying solid polymer formulations with increasing PEG 600 content added to the pre-formulated solid polymer formulations are shown in Table 3 and plotted in FIG. 8.

TABLE 3
DSC Melt Data And Viscosities Of PLG Acid-Initiated
56 kDa Solid Polymer Formulations With PEG 600

| | | | | DSC Melt | DSC Melt | Viscosity | Viscosity at |
|---|---|---|---|---|---|---|---|
| Formulation | Polymer | NMP | PEG 600 | Onset | Peak | at 5° C. | 25° C. |
| No. | (%) | (%) | (%) | Temp (° C.) | Temp (° C.) | (cP) | (cP) |
| 1 | 29 | 56 | 15 | N/A | N/A | 10,416 | 2,072 |
| 2 | 27 | 51 | 22 | −13.4 | −6.9 | 9,717 | 1,542 |
| 3 | 23 | 46 | 31 | −14.9 | −5.2 | 8,410 | 1,694 |
| 4 | 20 | 40 | 40 | −12.44 | 4.0 | 6,490 | 1,410 |
| 5 | 17 | 33 | 50 | −6.6 | 7.8 | 4,663 | 1,005 |
| 6 | 13 | 26 | 61 | −1.3 | 10.3 | 167,064 | 677 |

*N/A: Data not available

When compared to the suitable DSC melt profile criteria set for the solidifying liquid polymer formulation, i.e., No. 17 as seen in Table 1 of Example 2 above, none of the solidifying solid polymer formulations demonstrated a similar suitable Melt Onset Temperature and Melt Peak Temperature profile. All tested formulations showed DSC profiles with lower Melt Onset and Melt Peak Temperatures outside of the previously described suitable range. However, while useful, the Melt Onset and Melt Peak Temperatures are not the definitive criteria for identifying a suitable formulation. Instead, the viscosity at the storage and administration temperatures should also be considered when identifying suitable formulations. For all tested formulations, the viscosity was generally at about 2,000 cP or less when the formulations were analyzed at about 25° C. These viscosities are considered suitable for administration as a flowable composition via injection. Upon cooling to about 5° C., all of the tested formulations demonstrated viscosities of about 5,000 cP or greater. The viscosity remained generally stable at about 5,000 cP to about 10,000 cP as increasing amounts of PEG 600 was added from 0% to about 50% of the formulation. However, once the amount of PEG 600 added exceeded 50% of the formulation, the viscosity at 5° C. dramatically increased to in excess of 167,000 cP. When warmed back to 25° C. and reanalyzed, the viscosity of this sample with PEG 600 in excess of 50% of the formulation exhibited a low viscosity of about 600 cP, which is similar to the low viscosities exhibited by the other formulations (i.e., less than about 2,000 cP).

As an illustrative, but non-limiting, example, test formulation No. 6, when compared to the DSC profile of the solidifying liquid polymer control appears to be an unacceptable formulation with a Melt Onset and Melt Peak Temperature at −1.3° C. and 10.3° C., respectively. On the basis of Melt Onset Temperature, one could wrongly interpret that formulation No. 6 begins to melt during the cold storage temperatures in a clinical setting (i.e., at about 0° C. to about 8° C.) and thus may lead to a loss of dose uniformity while in storage. However, as shown, test formulation No. 6, despite a low Melt Onset Temperature at −1.3° C., the formulation itself remains extremely viscous at 5° C. at 167,064 cP. As such, test formulation No. 6 is still a suitable solidifying formulation for use in a clinical setting as the high viscosity at the cold storage temperatures of about 0° C. to about 8° C. prevents API movement during storage even though the formulation begins to experience its Melt Onset Temperature, and the formulation by about 18° C. to about 25° C. is a flowable, low viscous suspension ready for administration by injection into a patient in need thereof.

### Example 6: Preparation of Temperature Sensitive High-Viscosity Poly(D,L-Lactide-Co-Glycolide) Solid Polymer Formulations with PEG 1000 Added after Formulation

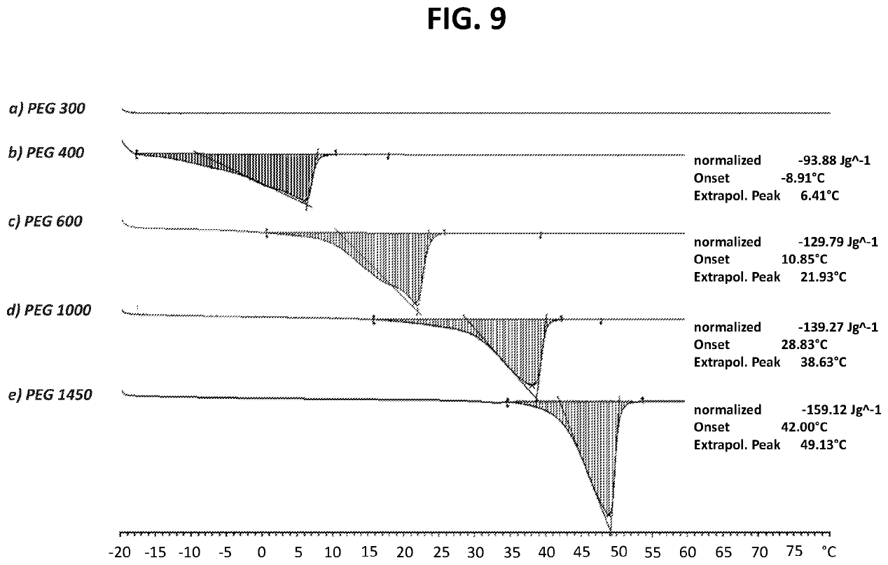

The results from Example 5 indicated that adding PEG 600 to pre-formulated solid polymers can be used to reduce the content of PEG 600 as a percentage of the solvent (i.e., compared to the formulations in Example 4); however, while still demonstrating suitable solidification properties, an excessive amount of PEG 600 was required. As different molecular weight PEGs possess different melting temperatures, it was thought that higher and lower molecular weight PEGs, other than or in addition to PEG 600, could be used to tune the melt profiles of the solid polymer formulation. FIG. 9 shows the DSC melt profiles of various sized PEGs. As the size of the PEG increases, the melt profile shift to higher temperatures and this may offer the ability to tune the melt profile of the solid polymer formulations.

PEG 1000 was selected for analysis to understand how the amount of a larger PEG effects solidification of the solid polymer formulation. Increasing amounts of PEG 1000 was added directly to syringes containing a pre-formulated solid polymer formulation comprising, by weight percentage, 34% of a 56 kDa 50:50 PLG acid-initiated polymer and 66% NMP. Specifically, 26 mg, 58 mg, 99 mg, 162 mg, 202 mg, 223 mg, or 341 mg of PEG 1000 was added to the pre-formulated solid polymer formulation to achieve a PEG 1000 content of 7%, 14%, 22%, 32%, 37%, 39%, and 50%, respectively, by weight of the formulation. The formulations were mixed as described in the previous examples.

From visual observations, addition of PEG 1000 at an amount of 7% or more of the formulation the solid polymer formulation, resulted in solidification at refrigeration temperatures. However, complete solidification, as measured by resistance to physical disruption via prodding with a syringe needle within the syringe chamber, did not occur until PEG 1000 was added at an amount of at least 39% or more of the formulation. Samples with PEG 1000 content from 7% to 32% of the formulation, demonstrated solidification upon freezing at 5° C. but could still be mechanically disrupted into a frozen-slush state when prodded with a needle that was inserted into the syringe.

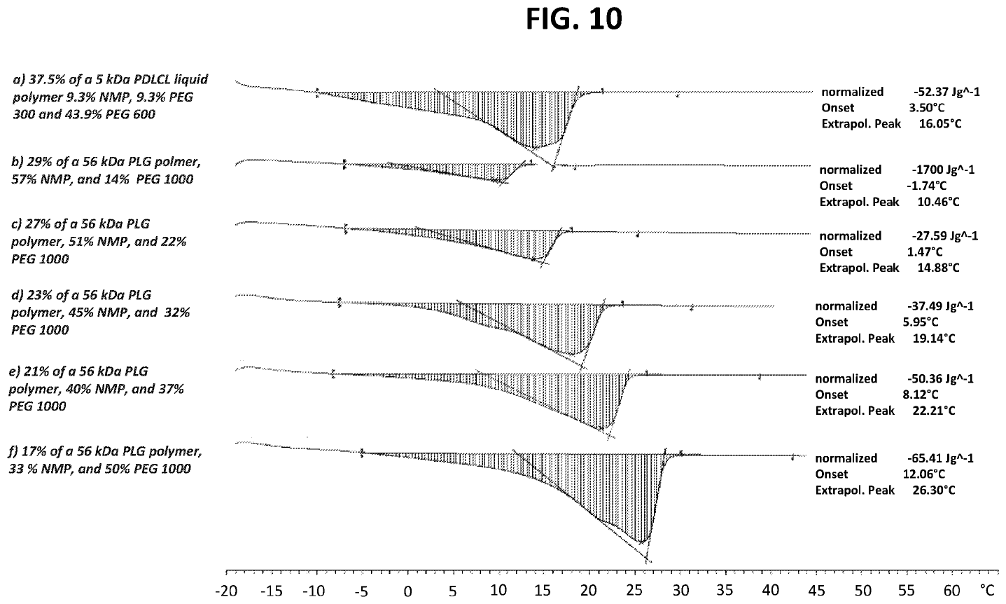

DSC melt and viscosity analysis of these samples was performed. The DSC melt profiles for these samples are shown in FIG. 10. As a reference, a liquid polymer formulation comprising, by weight percentage, 37.5% of a 5 kDa 75:25 PDLCL glycolic acid-initiated polymer, 9.3% NMP, 9.3% PEG 300, and 43.9% PEG 600 (formulation No. 17 from Table 1 in Example 2) is also shown in FIG. 10. This reference demonstrated a DSC Melt Onset Temperature and Melt Peak Temperature of 3.88° C. and 16.05° C., respectively. The DSC data and measured viscosity of these formulations are provided in Table 4 below.

TABLE 4
DSC Melt Data And Viscosities Of PLG Acid-Initiated
56 kDa Solid Polymer Formulations With PEG 1000

| | | | PEG | DSC Melt | DSC Melt | Viscosity | Viscosity |
|---|---|---|---|---|---|---|---|
| | Polymer | NMP | 1000 | Onset | Peak Temp | at 5° C. | at 25° C. |
| Formulation No. | (%) | (%) | (%) | Temp (° C.) | (° C.) | (cP) | (cP) |
| 1 | 32 | 61 | 7 | N/A | N/A | N/A | N/A |
| 2 | 29 | 57 | 14 | −1.7 | 10.5 | N/A | N/A |
| 3 | 27 | 51 | 22 | 1.5 | 14.9 | 10,049 | 2,991 |
| 4 | 23 | 45 | 32 | 6.0 | 19.1 | 15,402 | 2,514 |
| 5 | 21 | 42 | 37 | 8.1 | 22.2 | N/A | N/A |
| 6 | 21 | 40 | 39 | 8.8 | 22.5 | N/A | N/A |
| 7 | 17 | 33 | 50 | 12.1 | 26.3 | N/A | N/A |

*N/A: Data not available

Increasing amounts of PEG 1000 added to the solid polymer formulations corresponded with an upward shift of the Peak Onset and Peak Melt Temperatures. When compared to the melt profile of a solidifying liquid polymer formulation containing PEG 600, the addition of PEG 1000 at an amount of 22% to 32% of the formulation demonstrated a suitable Melt Onset Temperature and Melt Peak Temperature range similar to that seen for the reference solidifying liquid polymer formulation No. 17 in Table 1 in Example 2. When the amount of PEG 1000 was at 22% of the formulation, the Melt Onset Temperature and Melt Peak Temperature occurred at 1.5° C. and 14.9° C., respectively. The viscosity of this formulation at 5° C. was 10,049 cP but was lowered to 2,991 cP when the formulation was warmed to 25° C. When the amount of PEG 1000 was at 32% of the formulation, the Melt Onset Temperature and Melt Peak Temperature occurred at 6.0° C. and 19.1° C., respectively. The viscosity of this formulation at 5° C. was 15,402 cP but was lowered to 2,514 cP when the formulation was warmed to 25° C. As such, test formulation Nos. 3 and 4 are suitable solidifying formulations for use in a clinical setting as they possess high viscosity at the cold storage temperatures of about 0° C. to about 8° C. to prevent API movement during storage but then convert to a low viscosity, flowable composition ready for injection at about room temperature of 25° C.

### Example 7: Preparation of Temperature Sensitive High-Viscosity Poly(D,L-Lactide-Co-Glycolide) Solid Polymer Formulations with PEG 1450 Added after Formulation

PEG 1450 was selected for further analysis to understand how the amount of larger PEG effects solidification of the solid polymer formulation. Increasing amounts of PEG 1450 was added directly to syringes containing a pre-formulated solid polymer formulation comprising, by weight percentage, 34% of a 56 kDa 50:50 PLG acid-initiated polymer and 66% of NMP. Specifically, 98 mg or 150 mg of PEG 1450 was added to the pre-formulated solid polymer formulation to achieve a PEG 1450 content at 22% and 30% by weight of the formulation and the formulations were mixed as described in the previous examples.

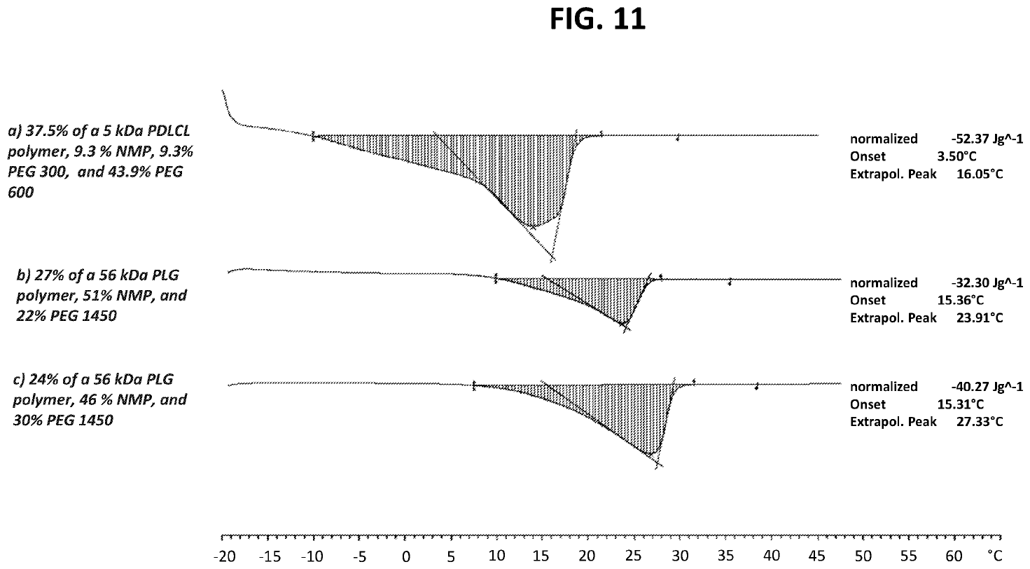

From visual observations, addition of PEG 1450 at the lowest tested amount of 22% of the formulation resulted in solidification at refrigeration temperatures. DSC melt and viscosity analysis of these samples was performed. The DSC melt profiles for these samples is shown in FIG. 11. As a reference, a liquid polymer formulation comprising, by weight, 37.5% of a 5 kDa 75:25 PDLCL glycolic acid-initiated polymer, 9.3% NMP, 9.3% PEG 300, and 43.9% PEG 600 was included (formulation No. 17 from Table 1 in Example 2). This reference demonstrated a DSC Melt Onset Temperature and Melt Peak Temperature of 3.88° C. and 16.05° C., respectively. The DSC data and measured viscosity of these formulations are provided in Table 5 below.

TABLE 5
DSC Melt Data And Viscosities Of PLG Acid-Initiated
56 kDa Solid Polymer Formulations With PEG 1450

| | | | PEG | DSC Melt | DSC Melt | Viscosity | Viscosity |
|---|---|---|---|---|---|---|---|
| Formulation | Polymer | NMP | 1450 | Onset | Peak Temp | at 5° C. | at 25° C. |
| No. | (%) | (%) | (%) | Temp (° C.) | (° C.) | (cP) | (cP) |
| 1 | 27 | 51 | 22 | 15.4 | 23.9 | 22,990 | 3,723 |
| 2 | 24 | 46 | 30 | 15.3 | 27.3 | 29,827 | 7,583 |

Increasing amounts of PEG 1450 added to the solid polymer formulations corresponded with an upward shift of the Peak Onset and Peak Melt Temperatures. When compared to the melt profile of a solidifying liquid polymer formulation containing PEG 600, the addition of PEG 1450 to 22% to 30% of the solid polymer formulation demonstrated a Melt Onset Temperature and Melt Peak Temperature range slightly above that of the reference solidifying liquid polymer formulation. When the amount of PEG 1450 was at 22% of the formulation, the Melt Onset Temperature and Melt Peak Temperature occurred at 15.4° C. and 23.9° C., respectively. The viscosity of this formulation at 5° C. was 22,990 cP but was lowered to 3,723 cP when the formulation was warmed to 25° C. When PEG 1450 was at 30% of the formulation, the Melt Onset Temperature and Melt Peak Temperature occurred at 15.3° C. and 27.9° C., respectively. The viscosity of this formulation at 5° C. was 29,827 cP but then decreased to 7,583 cP when the formulation was warmed to 25° C. As such, test formulation No. 1, is a suitable solidifying formulation for use in a clinical setting as it possesses high viscosity at the cold storage temperatures of about 0° C. to about 8° C. to prevent API movement during storage but then convert to a low viscosity, flowable composition ready for injection at about room temperature of 25° C.

### Example 8: Preparation of Temperature Sensitive High-Viscosity Poly(D,L-Lactide-Co-Glycolide) Solid Polymer Formulations with PEG 3350 Added after Formulation

PEG 3350 was selected for further analysis to understand how the amount of larger PEG effects solidification of the solid polymer formulation. Increasing amounts of PEG 3350 was added directly to syringes containing a pre-formulated solid polymer formulation comprising, by weight percentage, 34% of a 56 kDa 50:50 PLG acid-initiated polymer and 66% of NMP. An amount of 15.7 mg or 22.3 mg of PEG 3350 was added to the pre-formulated solid polymer formulation to achieve a PEG 3350 content at 4.4% and 6.0% of the formulation, respectively.

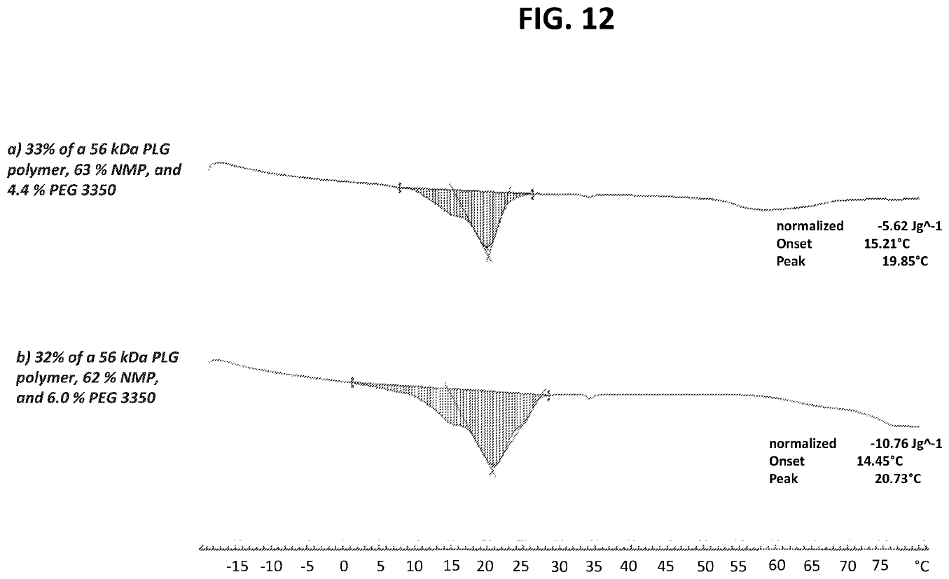

DSC melt and viscosity analysis of these samples was performed. The DSC melt profiles for these samples is shown in FIG. 12. The DSC data and measured viscosity of these formulations are provided in Table 6 below.

TABLE 6
DSC Melt Data Of PLG Acid-initiated 56 kDa Solid Polymer
Formulations With PEG 3350

| | | | PEG | DSC Melt | DSC Melt |
|---|---|---|---|---|---|
| Formulation | Polymer | NMP | 3350 | Onset Temp | Peak Temp |
| No. | (%) | (%) | (%) | (° C.) | (° C.) |
| 1 | 33 | 63 | 4.4 | 15.2 | 19.9 |
| 2 | 32 | 62 | 6.0 | 14.5 | 20.7 |

*N/A: Data not available

The addition of PEG 3350 to 4.4% or 6.0% of the solid polymer formulation demonstrated a Melt Onset Temperature and Melt Peak Temperature range slightly above that of the reference solidifying liquid polymer formulation. When the amount of PEG 3350 was at 4.4% of the formulation, the Melt Onset Temperature and Melt Peak Temperature occurred at 15.2° C. and 19.9° C., respectively. When the amount of PEG 3350 was 6.0% of the formulation, the Melt Onset Temperature and Melt Peak Temperature occurred at 14.5° C. and 20.7° C., respectively. As such, test formulation Nos. 1 and 2 are suitable solidifying formulations for use in a clinical setting as they possess high viscosity at the cold storage temperatures of about 0° C. to about 8° C. to prevent API movement during storage but then convert to a low viscosity, flowable composition ready for injection at about room temperature of 25° C.

### Example 9: Preparation of Temperature Sensitive High-Viscosity Poly(D,L-Lactide-Co-Glycolide) Solid Polymer Formulations

Increasing amounts of PEG 600 were added directly to syringes containing a pre-formulated solid polymer formulation comprising, by weight, 50% of a 25.5 kDa 85:15 PLG hexanediol-initiated solid polymer, either 30%, 35%, 40%, or 45% of PEG 600, and NMP sufficient so that all parts total to 100%. Additionally, two formulations of the same solid polymer were prepared. The first of these two comprises, by weight, 50% of a 25.5 kDa 85:15 PLG hexanediol-initiated solid polymer, 7.5% NMP, 7.5% PEG 300, and 35% PEG 600. The second of these two comprises, by weight, 37.5% of a 25.5 kDa 85:15 PLG hexanediol-initiated solid polymer, 9.4% NMP, 9.4% PEG 300, and 43.8% PEG 600. DSC and viscosity analysis of these samples was then performed, and the results are summarized in Table 7 below.

TABLE 7
DSC Melt Data Of PLG Hexanediol-initiated
25.5 kDa Solid Polymer Formulations

| | | | | | DSC Melt | DSC Melt |
|---|---|---|---|---|---|---|
| | | | | | Onset | Peak |
| Formulation | Polymer | NMP | PEG 300 | PEG 600 | Temp | Temp |
| No. | (%) | (%) | (%) | (%) | (° C.) | (° C.) |
| 1 | 50.0 | 20.0 | — | 30.0 | −6.48 | 9.56 |
| 2 | 50.0 | 15.0 | — | 35.0 | −0.69 | 12.40 |
| 3 | 50.0 | 10.0 | — | 40.0 | 3.82 | 15.45 |
| 4 | 50.0 | 5.0 | — | 45.0 | 7.56 | 17.39 |
| 5 | 50.0 | 7.5 | 7.5 | 35.0 | 1.00 | 14.22 |
| 6 | 37.5 | 9.4 | 9.4 | 43.8 | 5.74 | 14.79 |

*N/A: Data not available

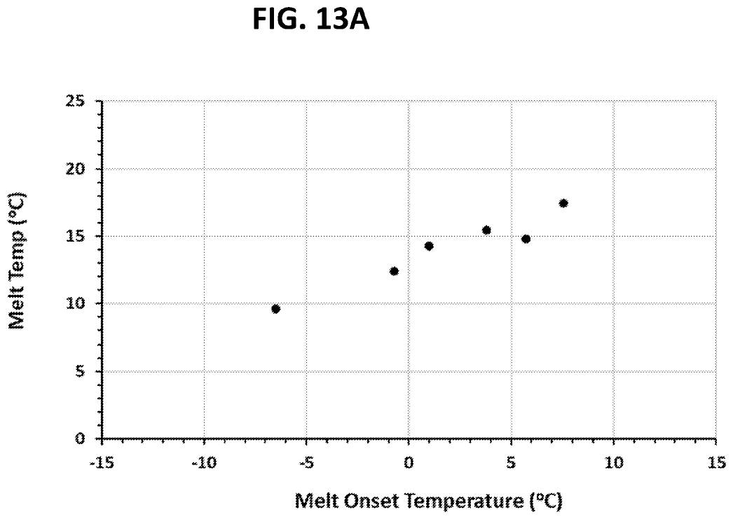

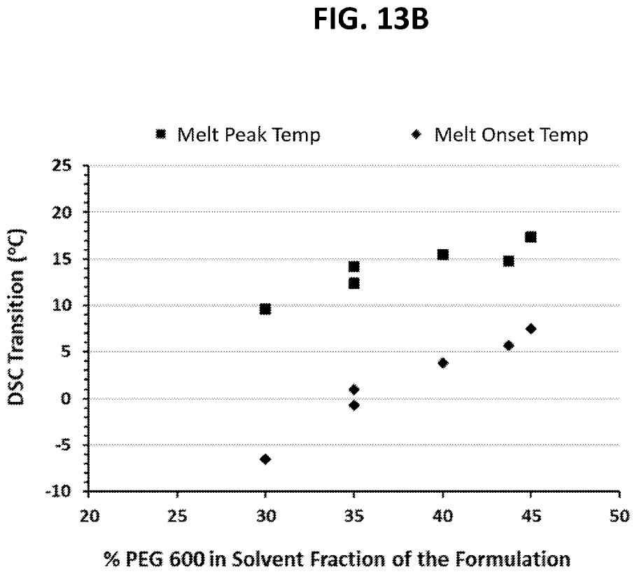

The Melt Onset Temperature versus Melt Peak Temperature for the various solidifying formulations of Table 7 are plotted in FIG. 13A (⋅). The DSC transition temperature as a function of the percentage of PEG 600 within the formulation are plotted in FIG. 13B. The Melt Onset Temperatures are shown by the diamonds (♦) and the Melt Peak Temperatures are shown on the plots by the squares (▪). DSC data analysis identified solid polymer formulations with Melt Onset temperature of about 8° C. or higher and a Melt Peak temperature of about 20° C. or less.

### Example 10: Preparation of a Temperature Sensitive High-Viscosity Poly(L,D-Caprolactone-Co-Lactide) Liquid Polymer Formulation with Different Solvents

To evaluate the effects that solvent identity and ratio have upon the solidification of liquid polymer formulations, test formulations comprising, by weight percentage, 37.5% of a 5 kDa 75:25 PDLCL glycolic acid-initiated liquid polymer, either 0%, 6.0%, 37.5%, or 50% of PEG 600, and various amounts of either NMP, DMSO, BnBzO, or CRODASOL™, or combinations thereof, such that all parts by weight percentage total to 100% were prepared. DSC and viscosity analysis of these samples was then performed, and the results are summarized in Table 8.

TABLE 8
DSC Melt Data of PDLCL Acid-Initiated 5 kDa Liquid Polymer
Formulations With Different Solvents

| | | | | DSC Melt | DSC Melt |
|---|---|---|---|---|---|
| Formulation | Polymer | | | Onset Temp | Peak Temp |
| No. | (%) | Solvent (%) | Solvent Composition | (° C.) | (° C.) |
| 1 | 37.5 | 62.5 | 60:40 CRODASOLT:NMP | 1.09 | 13.41 |
| 2 | 37.5 | 62.5 | 70:30 CRODASOLT:NMP | −2.51 | 17.44 |
| 3 | 37.5 | 62.5 | 80:20 CRODASOLT:NMP | 2.35 | 20.16 |
| 4 | 37.5 | 62.5 | 80:20 peg 600:BnBzO | 6.35 | 14.94 |
| 5 | 37.5 | 62.5 | 60:40 PEG 600:BnBzO | 3.83 | 10.14 |
| 6 | 37.5 | 62.3 | 90:10 DMSO:PEG 600 | 2.93 | 11.39 |

CRODASOL™ was miscible in formulations Nos. 1-3 but higher CRODASOL™ content appeared to correlate with higher viscosity. In formulation No. 6, DMSO was observed to be predominantly crystalline at 5° C. Of the tested formulations, formulation No. 4 demonstrated the most suitable Melt Onset Temperature to Melt Peak temperature range wherein NMP was replaced with by BnBzO.

### Example 11: Preparation of a Temperature Sensitive High-Viscosity Poly(D,L-Lactide-Co-Glycolide) Solid Polymer Formulation with Different Solvents

To evaluate the effects that solvent identity and ratio have upon the solidification of solid polymer formulations, test formulations comprising, by weight, 34% of a 56 kDa 50:50 PLG acid-initiated solid polymer, either 0%, 6.6%, 39.6%, or 52.8% of PEG 600, and various amounts of either NMP, DMSO, BnBzO, CRODASOL™, or combinations thereof, such that all parts by weight percentage total to 100% were prepared. DSC and viscosity analysis of these samples was then performed and the results are summarized in Table 9.

TABLE 9
DSC Melt Data Of PLG Acid-Initiated 56 kDa Solid Polymer Formulations
With Different Solvents

| | | | | DSC Melt | DSC Melt |
|---|---|---|---|---|---|
| Formulation | Polymer | | | Onset Temp | Peak Temp |
| No. | (%) | Solvent (%) | Solvent Composition | (° C.) | (° C.) |
| 1 | 34.0 | 66.0 | 60:40 CRODASOLT:NMP | −5.82 | 13.57 |
| 2 | 34.0 | 66.0 | 80:20 CRODASOLT:NMP | 1.71 | 20.81 |
| 3 | 34.0 | 66.0 | 80:20 PEG 600:BnBzO | 6.41 | 15.38 |
| 4 | 34.0 | 66.0 | 60:40 PEG 600:BnBzO | Not Obs. | Not Obs. |
| 5 | 34.0 | 66.0 | 90:10 DMSO:PEG 600 | 4.85 | 12.34 |

CRODASOL™ was partly miscible in formulation No. 1 and appeared biphasic when visually inspected. In formulation No. 6, DMSO was observed to be predominantly crystalline at 5° C. Of the tested formulations, formulation Nos. 3 and 6 demonstrated the most suitable Melt Onset Temperature to Melt Peak temperature range wherein NMP was replaced with by BnBzO or DMSO.

### Example 12: Preparation of a Temperature Sensitive High-Viscosity Testosterone Undecanoate Poly(D,L-Lactide-Co-Glycolide) Solid Polymer Formulation with Different Solid Polymer Molecular Weight Sizes

To assess the effects that polymer size has upon the temperature sensitive solidification properties, test formulations comprising by weight, 20% of TU with particle size of D_{v,50}=67 μm, 30% of either a 5, 10, or 13 kDa 70:30 PLG acid-initiated polymer, 35% PEG 600, either 0% or 7.5% of PEG 300, and either 7.5% or 15% of NMP, such that all parts total 100%, were prepared. DSC and viscosity analysis of these samples was performed, and the results are summarized in Table 10.

TABLE 10
DSC Melt Data And Viscosities Of PLG Acid-Initiated Solid Polymer
Formulations With Different Polymer Molecular Weight Sizes

| | | | | | | | DSC Melt | DSC Melt | | |
|---|---|---|---|---|---|---|---|---|---|---|
| | | | Polymer | | PEG | PEG | Onset | Peak | Viscosity | Viscosity |
| Formulation | TU | Polymer | Size | NMP | 300 | 600 | Temp | Temp | at 5° C. | at 25° C. |
| No. | (%) | (%) | (kDa) | (%) | (%) | (%) | (° C.) | (° C.) | (cP) | (cP) |
| 1 | 20 | 30 | 5 | 15 | 0 | 35 | −3.29 | 10.74 | 20,063 | 3,225 |
| 2 | 20 | 30 | 5 | 7.5 | 7.5 | 35 | 0.62 | 13.29 | 60,477 | 6,301 |
| 3 | 20 | 30 | 10 | 15 | 0 | 35 | −3.01 | 10.29 | 40,071 | 6,581 |
| 4 | 20 | 30 | 10 | 7.5 | 7.5 | 35 | 0.36 | 12.49 | 96,759 | 11,038 |
| 5 | 20 | 30 | 13 | 15 | 0 | 35 | −1.54 | 9.62 | 77,655 | 13,478 |
| 6 | 20 | 30 | 13 | 7.5 | 7.5 | 35 | 2.76 | 12.84 | 186,226 | 25,011 |

Comparing test formulations Nos. 1, 3, and 5, shows the effects of increasing the molecular weight of the solid PLG polymer from 5 kDa to 10 kDa to 13 kDa. As the PLG solid polymer molecular weight size increases, so does the viscosity at both 5° C. and 25° C. When cooled to 5° C., all three test formulations are sufficiently viscous to prevent API movement during cold storage as the viscosities recorded are 20,063 cP, 40,071 cP, and 77,655 cP for the 5 kDa, 10 kDa, and 13 kDa sized PLG solid polymers used. When these formulations were warmed to 25° C., their recorded viscosities lowered to 3,225 cP, 6,301 cP, and 25,011 cP for the 5 kDa, 10 kDa, and 13 kDa PLG solid polymers, respectively. As such, on the basis of viscosity at 5° C. and 25° C., test formulation No. 1 would be suitable for clinical use. Test formulation Nos. 2 and 3 may be suitable as their viscosity at 25° C. is marginally higher at 6,301 cP and 6,581 cP, respectively. Test formulation Nos. 4 and 5 appear to be increasingly less suitable as their viscosities at 25° C. increases to 11,038 cP and 13,478 cP, respectively. Lastly, test formulation No. 6 is likely not suitable for clinical use as the viscosity remains rather high at 25,011 cP when at 25° C.

In general, increasing the molecular weight size of the PLG solid polymer within the formulation correlates with increasing viscosity at both 5° C. and 25° C. Similarly, the addition of PEG 300 also increases the viscosity of each of the three molecular weight size classes at both 5° C. and 25° C. Comparing test formulations No. 1 to 2, No. 3 to 4, and No. 5 to 6 shows that the addition of PEG 300 causes the viscosity to increase nearly 2-fold to 3-fold at 5° C. When warmed, these samples with PEG 300 retain a viscosity nearly 2-fold of their counterparts that lack PEG 300.

On the basis of the DSC melt data, all of the test formulations have low Melt Onset Temperatures. Although low, these Melt Onset Temperatures do not prevent these formulations from being useful in a clinical setting. Firstly, all of these test formulations still retain a Melt Peak Temperature of about 10° C. to about 13° C. As such, by 25° C., all will be sufficiently melted for administration by injection. However, of the aforementioned test formulations, only No. 1 and potentially Nos. 2 and 3, possess a low enough viscosity which would be generally considered suitable for said injection. All of the test formulations remain highly viscous at 5° C. such that API should not move during prolong long term storage. As such, these formulations may be useful despite beginning to melt at lower temperatures as seen by their Melt Onset Temperatures as low as about −3° C. For instance, as a non-limiting example, test formulation No. 1 may begin to thaw at −3.29° C. but it remains highly viscous at 5° C. with a viscosity at 20,063 cP. It is then fully melted by 10.74° C. and has a viscosity of 3,225 cP by 25° C. As such, it may be useful under some clinical settings.

### Example 13: Testosterone Undecanoate In Vitro Release from Temperature Sensitive High-Viscosity Testosterone Undecanoate Poly(L,D-Caprolactone-Co-Lactide) Liquid Polymer Formulations

The in vitro release profiles of solidifying liquid polymer formulations containing TU were conducted using jar technique method with 1% CTAB surfactant in TRIS buffer, pH 8.7 at 37° C. Depots were made by injecting 200 mg of formulation into 200 mL release media. Samples were then periodically collected and analyzed for released drug content. Solidifying liquid polymer formulations comprising, by weight, 20% TU, 30% of a 14.2 kDa 75:25 PDLCL acid-initiated liquid polymer, 35% PEG 600, with or without PEG 300 up to 7.5%, and NMP from 7.5% to 15%, such that all parts total to 100%, were prepared. Non-formulated TU with a particle size of 67 μm (i.e., with no polymer) were also analyzed as a reference. Additionally, a 20% TU non-solidifying liquid polymer comprising 30% of a 14.2 kDa PDLCL liquid polymer with 25% NMP and 25% PEG 300 was prepared as a reference. The composition of these tested solidifying liquid polymer formulations are summarized in Table 11.

TABLE 11
Dissolved TU Drug Content And Viscosity For TU Solidifying
14.2 kDa PDLCL Acid-Initiated Liquid Polymer Formulations

| | | | | PEG | PEG | Dissolved TU | Viscosity | Viscosity |
|---|---|---|---|---|---|---|---|---|
| Formulation | TU | Polymer | NMP | 300 | 600 | (% within | at 5° C. | at 25° C. |
| No. | (%) | (%) | (%) | (%) | (%) | formulation) | (cP) | (cP) |
| 1 | 20 | 30.0 | 7.5 | 7.5 | 35.0 | 0.6 | N/A | 8,454 |
| 2 | 20 | 30.0 | 15.0 | N/A | 35.0 | 0.8 | N/A | 3,985 |

*N/A: Data not available

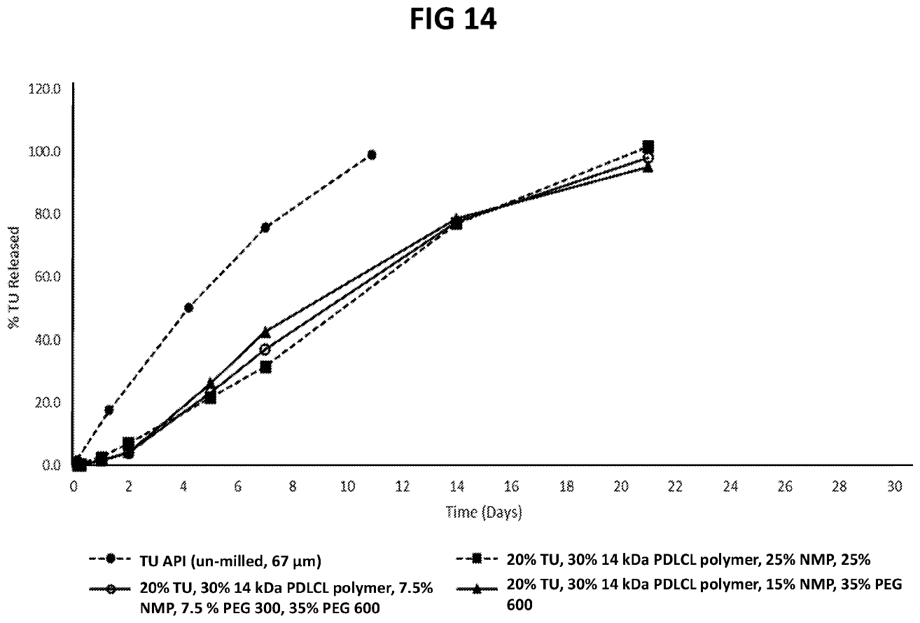

FIG. 14 shows the time release profile of TU from these solidifying liquid polymer formulations. The non-formulated TU particles, provided as control (⋅), undergo fairly rapid release with approximately about 75% of TU being released within the first 7 days. Comparatively, both of the test formulations, No. 1 (o) and No. 2 (▴) behaved similar to one another with a slower release profile. For both test formulations, about 30% to about 40% of the TU was released within the first 7 days. By 14 days, both test formulations had released about 75% of the TU. By 21 days, about 90% to about 100% of the TU had been released from both depots into the media. Of interest is that both solidifying test formulations have a near identical TU release profile as the non-solidifying TU liquid polymer formulation reference (▪). This identical TU release profile indicates that the addition of PEG 600, which helps to achieve temperature sensitive solidification characterized by high viscosity at temperatures from about 0° C. to about 8° C. to prevent API movement but which reverts to a low viscosity by temperatures at about 18° C. to about 25° C., does not significantly affect the targeted pharmacological release characteristics of the associated TU. This is potentially advantageous as existing formulations with known pharmacokinetics can be prepared as a solidifying variant with the expectation that the targeted pharmacokinetics will remain essentially unaltered, thereby, minimizing any efforts needed to re-optimize the formulation.

The viscosities of each test formulation are also reported in Table 11. Test formulation No. 1 demonstrated higher viscosity at 25° C. than that of test formulation No. 2. This is due to the additional presence of PEG 300. Test formulation No. 2, which contains PEG 600 for solidification, retains a suitable viscosity of 3,985 cP at 25° C. As such, test formulation No. 2 is a suitable solidifying formulation for use in a clinical setting as it possesses high viscosity at the cold storage temperatures of about 0° C. to about 8° C. to prevent API movement during storage but which then converts to a low viscosity, flowable composition ready for injection at about room temperature of 25° C.

### Example 14: Testosterone Cypionate In Vitro Release from Temperature Sensitive High-Viscosity Testosterone Cypionate Poly(L,D-Caprolactone-Co-Lactide) Liquid Polymer Formulations

The in vitro release profiles of solidifying liquid polymer formulations containing testosterone cypionate (TC) were conducted using jar technique method with 1% CTAB surfactant in TRIS, pH 8.7 at 37° C. Depots were made by injecting 200 mg of formulation into 200 mL release media. Samples were then periodically collected and analyzed for release drug content. Solidifying liquid polymer formulations comprising, by weight, 20% TC, 30% of a 14.2 kDa 75:25 PDLCL acid-initiated liquid polymer, 35% PEG 600, with or without PEG 300 up to 7.5%, and NMP from 7.5% to 15%, such that all parts total to 100%, were prepared. Non-formulated TC with a particle size of 41 μm were also analyzed as a reference. Additionally, a 20% TC non-solidifying liquid polymer formulation comprising 30% of a 14.2 kDa 75:25 PDLCL liquid polymer with 25% NMP and 25% PEG 300 was prepared as a control. The composition of these tested solidifying liquid polymer formulations are summarized in Table 12.

TABLE 12
Dissolved TC Drug Content and Viscosity for TC Solidifying
14 kDa PDLCL Acid-Initiated Liquid Polymer Formulations

| | | | | PEG | PEG | Dissolved TC | Viscosity | Viscosity |
|---|---|---|---|---|---|---|---|---|
| | TC | Polymer | NMP | 300 | 600 | (% within | at 5° C. | at 25° C. |
| Formulation No. | (%) | (%) | (%) | (%) | (%) | formulation) | (cP) | (cP) |
| 1 | 20 | 30.0 | 7.5 | 7.5 | 35.0 | 1.8 | N/A | 6,540 |
| 2 | 20 | 30.0 | 15.0 | N/A | 35.0 | 2.3 | N/A | 3,617 |

*N/A: Data not available

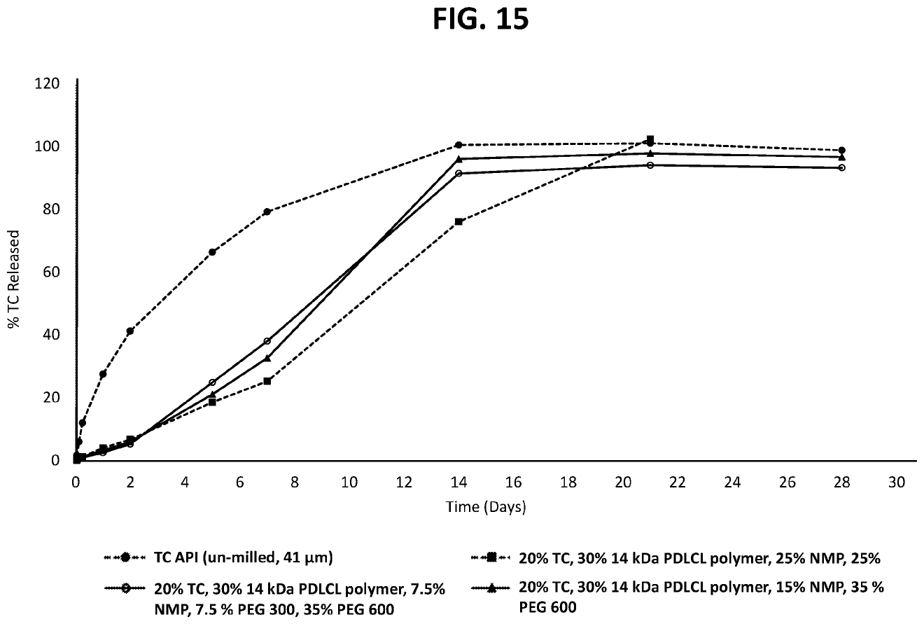

FIG. 15 shows the time release profile of TC from these solidifying liquid polymer test formulations. Non-formulated TC particles (⋅), provided as a reference, undergo fairly rapid release with approximately about 80% of TC being released within the first 7 days. Comparatively, both of the test formulations, No. 1 (o) and No. 2 (▴), behaved similar to one another with a slower release profile. For both test formulations, about 30% to about 40% of the TC was released within the first 7 days. By 14 days, both test formulations had released about 90% to about 95% of the TC. By 21 days, about 100% of the TC had been released from both depots into the media. In contrast to the in vitro release profiles seen for TU from the solidifying liquid polymer formulations of Example 13, the in vitro release profile of TC from these solidifying liquid polymer formulations differs from the TC release profile observed for the non-solidifying control formulation (▪). The TC solidifying formulations of Table 12 release TC slightly faster than the corresponding TC non-solidifying reference formulation. The TC release profile is near identical amongst the formulations for about the first week. However, at around day 7, the solidifying formulations begin to release about 10% more TC than the non-solidifying control formulation. By day 14, the solidifying formulations have release about 15% to about 20% more TC than the corresponding non-solidifying control. By day 14 or shortly thereafter, the solidifying formulations have released all of the TC. Conversely, the TC non-solidifying control formulation does not finish releasing TC until around day 21.

The viscosities of each test formulation are also reported in Table 12. Similar to that seen for the TU solidifying formulations of Table 11 in Example 13, test formulation No. 1 demonstrated higher viscosity at 25° C. than that of test formulation No. 2. This is due to the additional presence of PEG 300. Test formulation No. 2, which contains PEG 600 for solidification, retains a suitable viscosity of 3,617 cP at 25° C. The viscosities of both formulations at 25° C. are slightly lower than the corresponding TU solidifying formulations used in Example 13. Test formulation No. 2 is suitable solidifying formulations for use in a clinical setting as it possesses high viscosity at the cold storage temperatures of about 0° C. to about 8° C. to prevent API movement during storage but which then converts to a low viscosity, flowable composition ready for injection at about room temperature of 25° C.

### Example 15: Testosterone Cypionate In Vitro Release from Temperature Sensitive High-Viscosity Testosterone Cypionate Poly(L,D-Caprolactone-Co-Lactide) Liquid Polymer Formulations with Different Molecular Weight Sizes

The in vitro release profiles of solidifying liquid polymer formulations containing testosterone cypionate (TC) but comprising different molecular weight PDLCL liquid polymer were conducted using jar technique method with 1% surfactant in phosphate buffered saline (PBS) at 37° C. Depots were made by injecting 200 mg of formulation into 200 mL release media. Samples were then periodically collected and analyzed for release drug content. A first solidifying liquid polymer formulation comprising, by weight percentage, 20% TC, 30% of a 10 kDa 75:25 PDLCL acid-initiated liquid polymer, 7.5% NMP, 7.5% PEG 300, and 35% PEG 600 was prepared. For comparison, a second solidifying liquid polymer formulation comprising, by weight percentage, 20% TC, 30% of a 14.2 kDa 75:25 PDLCL acid-initiated liquid polymer, 7.5% NMP, 7.5% PEG 300, and 35% PEG 600 was prepared. Non-formulated TC with a particle size of 41 μm was analyzed as a reference. Additionally, a 20% TC non-solidifying liquid polymer formulation comprising 30% of a 14.2 kDa 75:25 PDLCL liquid polymer with 25% NMP and 25% PEG 300 was included as a control. The compositions of the solidifying liquid polymer formulations are summarized in Table 13.

TABLE 13
Dissolved Drug Content And Viscosity For TC Solidifying
PDLCL Acid-Initiated Liquid Polymer Formulations

| | | | | | | | Dissolved | | |
|---|---|---|---|---|---|---|---|---|---|
| | | | Polymer | | PEG | PEG | TC (% | Viscosity | Viscosity |
| Formulation | TC | Polymer | Size | NMP | 300 | 600 | within | at 5° C. | at 25° C. |
| No. | (%) | (%) | (kDa) | (%) | (%) | (%) | formulation) | (cP) | (cP) |
| 1 | 20 | 30.0 | 10.0 | 7.5 | 7.5 | 35.0 | N/A | N/A | 3,811 |
| 2 | 20 | 30.0 | 14.2 | 7.5 | 7.5 | 35.0 | 1.8 | N/A | 6,540 |

*N/A: Data not available

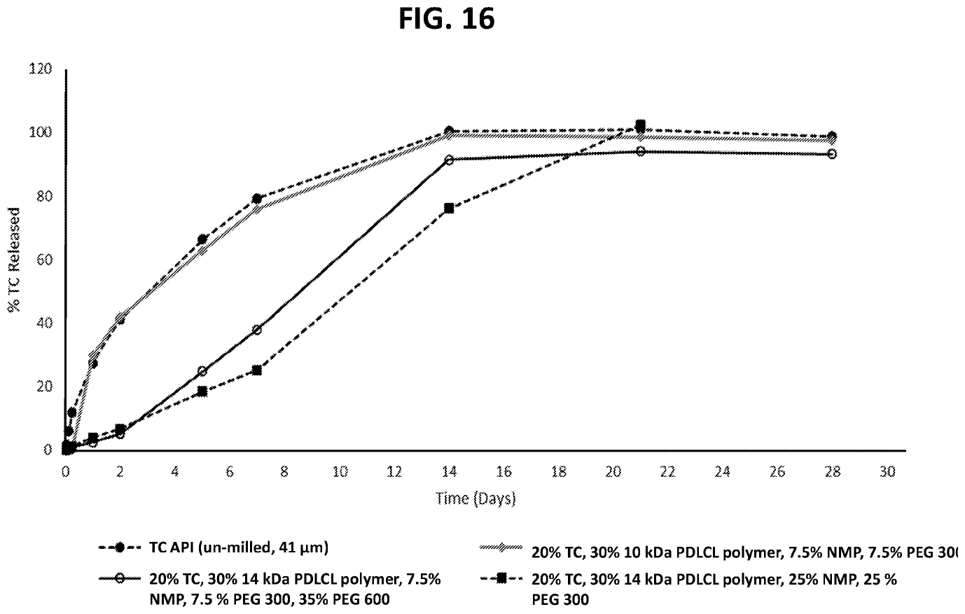

FIG. 16 shows the time release profile of TC from these solidifying liquid polymer test formulations. Non-formulated TC particles (⋅), provided as control, undergo fairly rapid release with approximately about 80% of TC being released within the first 7 days. Notably, for the TC release profile of test formulation No. 1 (grey ♦) comprising the 10 kDa PDLCL liquid polymer, the release profile was near identical to that of the unformulated TC nanoparticle control. However, as previously shown in the prior examples, the test formulation comprising the 14 kDa PDLCL liquid polymer (o) shows markedly slower release profile of TC but is not as slow as that seen for the non-solidifying counterpart (▪). However, when the viscosity at 25° C. was measured, test formulation No. 1 with the smaller 10 kDa PDLCL liquid polymer was less viscous than test formulation No. 2 comprising the larger 14.2 kDa PDLCL liquid polymer, i.e., 3,811 cP versus 6,540 cP, respectively.

To further evaluate the effect of polymer molecular weight size upon the in vitro release profile of TC, a third and fourth formulation were prepared but without the addition of PEG 300. The third solidifying liquid polymer formulation comprises, by weight, 20% TC, 30% of a 14.2 kDa 75:25 PDLCL acid-initiated liquid polymer, 15% NMP, and 35% PEG 600. For comparison, the fourth solidifying liquid polymer formulation comprises, by weight percentage, 20% TC, 30% of a 22 kDa 75:25 PDLCL acid-initiated liquid polymer, 15% NMP, and 35% PEG 600. Non-formulated TC with a particle size of 41 μm was included. Additionally, a 20% TC non-solidifying liquid polymer comprising 30% of a 75:25 14.2 kDa PDLCL liquid polymer with 25% NMP and 25% PEG 300 was included as a reference. The compositions of the solidifying liquid polymer formulations are summarized in Table 14.

TABLE 14
Dissolved Drug Content And Viscosity For TC Solidifying
PDLCL Acid-Initiated Liquid Polymer Formulations

| | | | | | | | Dissolved | | |
|---|---|---|---|---|---|---|---|---|---|
| | | | Polymer | | PEG | PEG | TC (% | Viscosity | Viscosity |
| Formulation | TC | Polymer | Size | NMP | 300 | 600 | within | at 5° C. | at 25° C. |
| No. | (%) | (%) | (kDa) | (%) | (%) | (%) | formulation) | (cP) | (cP) |
| 1 | 20 | 30.0 | 14.2 | 15.0 | N/A | 35.0 | 2.3 | N/A | 3,617 |
| 2 | 20 | 30.0 | 22.0 | 15.0 | N/A | 35.0 | N/A | N/A | 8,063 |

*N/A: Data not available

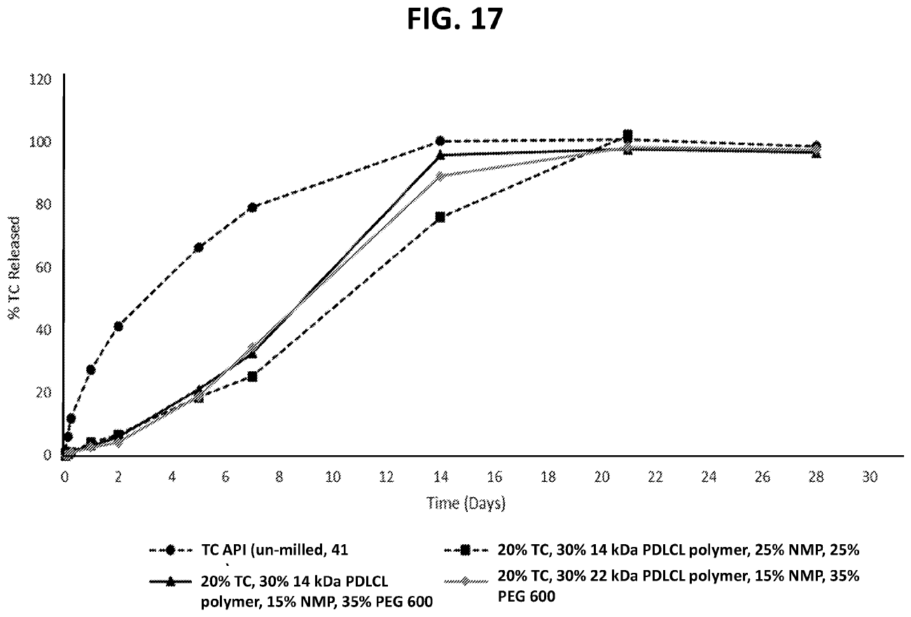

FIG. 17 shows the time releases profile of TC from this third (▴) and fourth (grey♦) solidifying liquid polymer test formulations. Non-formulated TC particles (⋅), provided as a reference, undergo fairly rapid release with approximately about 80% of TU being released within the first 7 days. Test formulation No. 1 possesses a near identical in vitro release profile of TC as that seen for test formulation No. 2 from FIG. 16 above, with the only difference between the formulations being the lack of PEG 300 in the latter test formulation. Furthermore, at 25° C., the viscosity for both of these 14.2 kDa 75:25 PDLCL liquid polymer formulations are relatively the same at 3,811 cP and 3,617 cP, respectively, with and without PEG 300. However, unlike the notable difference in the TC in vitro release profile seen with the 10 kDa 75:25 PDLCL liquid polymer formulation versus the 14.2 kDa PDLCL liquid polymer formulation of FIG. 16, there is little difference observed in the TC in vitro release profiles of the 14.2 kDa 75:25 PDLCL liquid polymer formulation versus the 22 kDa 75:25 PDLCL liquid polymer formulation, shown in FIG. 17. Although, neither formulation provides as long of a TC in vitro release profile as that seen with the 14.2 kDa non-solidifying PDLCL liquid polymer formulation. Like that seen in Table 13, the increasing polymer size of test formulation No. 2 leads to a higher viscosity at 25° C. of 8,063 cP over the smaller polymer size of test formulation No. 1. Therefore, in general, the molecular weight size of the PDLCL liquid polymer used within the solidifying formulation can affect the in vitro release pharmacokinetic properties of TC.

### Example 16: Preparation of a Temperature Sensitive High-Viscosity Poly(L,D-Caprolactone-Co-Lactide) Liquid Polymer Oil Suspension Formulation that Maintains Polymer-Oil Droplet Suspension Homogeneity

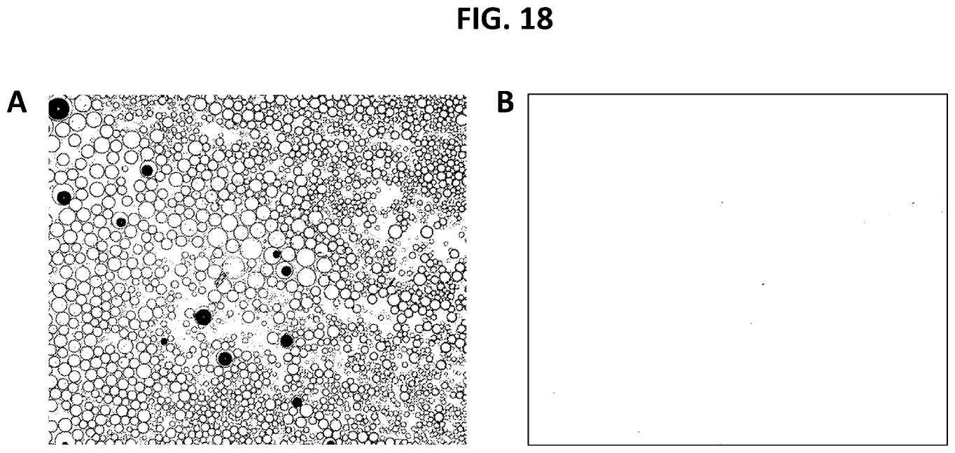

The ability to induce a suitable temperature sensitive solidification of a liquid polymer oil suspension was investigated. A liquid polymer oil suspension formulation comprising, by weight percentage, 30% of a 14 kDa 75:25 PDLCL polymer, 25% NMP, 25% PEG 300, and 20% mineral oil was prepared. This liquid polymer oil suspension was vigorously mixed via syringe-to-syringe and then imaged under 10× microscope magnification at room temperature. This sample was then frozen at 5° C. for 2 days before being subsequently re-warm to 25° C. where it was imaged again but without any vigorous mixing. Phase separation was verified when sample was viewed under magnification. As seen in FIG. 18A, there is a heterogeneous oil droplet field within the sample prior to being frozen at 5° C. However, as seen in FIG. 18B, this oil droplet field completely disappeared when the sample was frozen at 5° C. for 2 days and then subsequently warmed to 25° C. without any mixing.

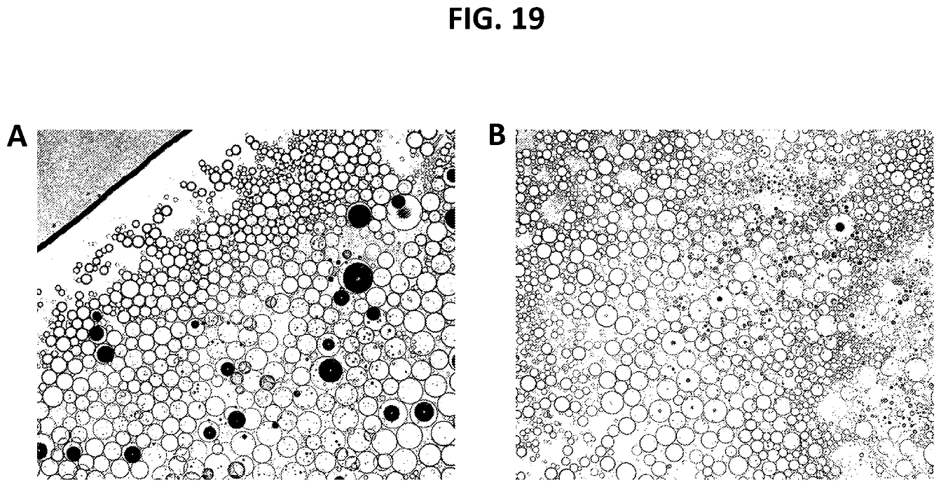

To maintain the oil droplet dispersion during freezing, PEG 300 within the polymer-oil formulation was replaced with PEG 600. A liquid polymer oil suspension formulation comprising, by weight percentage, 30% of a 14 kDa 75:25 PDLCL polymer, 15% NMP, 35% PEG 600, and 20% mineral oil was prepared, vigorously mixed, and then imaged under 10× microscope magnification at room temperature. This sample was then frozen at 5° C. for 2 days before being subsequently re-warmed to 25° C. where it was then re-imaged again but without any vigorous mixing. Addition of PEG 600 rendered the polymer-oil suspension with solidification properties such that freezing did not induce a polymer-oil phase separation as seen before. As seen in FIG. 19A, there is a heterogeneous oil droplet field within the sample prior to being frozen at 5° C. As seen in FIG. 19B, the liquid polymer-oil suspension maintained a heterogeneous oil droplet dispersion following thawing to 25° C. after being frozen for 2 days at 5° C. without any need to re-mix the suspension. However, when stored undisturbed at 25° C. for an additional 3 days, a visually apparent phase separation could readily be seen within the sample.

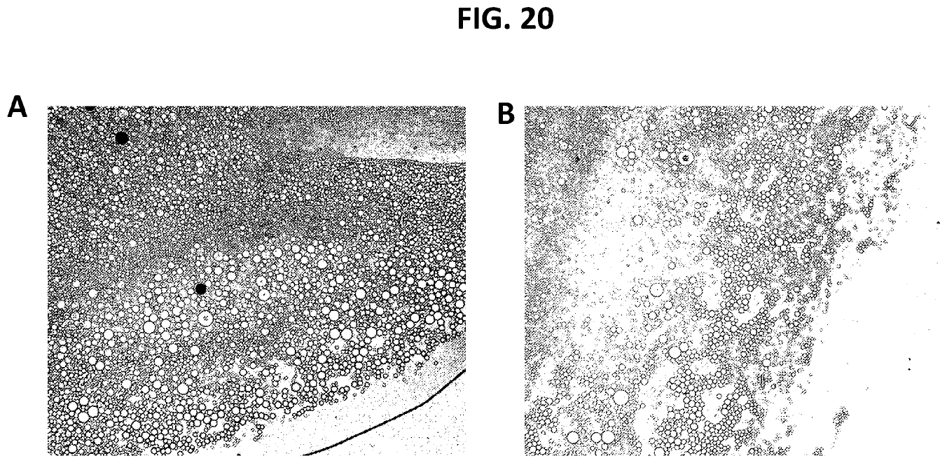

Additionally, instead of substituting PEG 600 in place of PEG 300, PEG 600 was added to a polymer-oil formulation. A liquid polymer oil suspension formulation comprising, by weight percentage, 30% of a 14 kDa 75:25 PDLCL polymer, 7.5% NMP, 7.5% PEG 300, 35% PEG 600, and 20% mineral oil was prepared. The sample was imaged at room temperature and again after freezing at 5° C. for 2 days similar to the other liquid polymer oil suspension formulations. Addition of PEG 600 rendered the polymer-oil suspension with solidification properties such that freezing did not induce a polymer-oil phase separation as seen before. As seen in FIG. 20A, there is a heterogeneous oil droplet field within the sample prior to being frozen at 5° C. As seen in FIG. 20B, the liquid polymer-oil suspension further comprising PEG 300 maintained a heterogeneous oil droplet dispersion following thawing to 25° C. after being frozen for 2 days at 5° C. without any need to re-mix the suspension. However, when stored undisturbed at 25° C. for an additional 3 days, a visually apparent phase separation could readily be seen within the sample.

### Example 17: Leuprolide Acetate Inhomogeneity within a Poly(D,L-Caprolactone-Co-Lactide) Liquid Polymer Formulation can not be Restored by Mixing

Leuprolide Acetate (LA), a synthetic nonapeptide analogue of gonadotropin-releasing hormone was another API was selected for analysis, similar to the use of testosterone in the previous examples. Conversely, to TU which is a hydrophobic steroid based molecule, LA is a hydrophilic peptide molecule with molecular weight of 1269.4 g/mol. Notably, unlike TU, LA is amorphous in nature. It has been shown that the crystalline nature of the API can impact how the API behaves within any given polymer formulation. Given the high solubility of LA within NMP, LA formulations were prepared using Benzyl Benzoate (BnBzO) in lieu of NMP, which was used as the solvent in previous examples where testosterone is the API. LA formulations using NMP as the solvent lead to a solution system rather than a suspension system. However, LA formulations in BnBzO form a suspension system, which helps assess API separation. The nature of LA as an API is different from TU in terms of its physical-chemical characteristics. Likewise, BnBzO based solvent system has different characteristics than NMP based solvent system. However, the physical stability of the suspension system is comparable regardless of the differences noted with the nature of API and the solvent system.

Separation of API particles from a liquid polymer-solvent system was observed to occur in syringes containing LA-liquid polymer formulation comprising, by weight, 12.0% LA, 30.8% of a 18.8 kDa 75:25 PDLCL-acid-initiated polymer, and 57.2% BnBzO, following simulated long-term storage using centrifugation model, which was used to demonstrate the physical instability of LA suspension which led to subsequent mixing to re-suspend the API after such simulated long-term storage. Separation of LA API particles from a liquid polymer-solvent system could be accomplished by centrifuging syringes containing the formulation at 2,000 rpm for 60 minutes at 4° C. (i.e., prolonged centrifuged simulated storage”).

To restore formulation homogeneity following LA particle separation from the liquid polymer-solvent system upon centrifuged simulated long-term storage, mixing studies were conducted. Centrifuged syringes containing the aforementioned non-solidifying LA-liquid polymer formulations were coupled to an empty Schott mixing syringe and then mixed back and forth for 0, 0.5, 5, 10, 15, or 25 mixing cycles. Each mixing cycle comprises a full depression and retraction of the syringe plunger rod. For each mixing cycle, samples were prepared in triplicate.

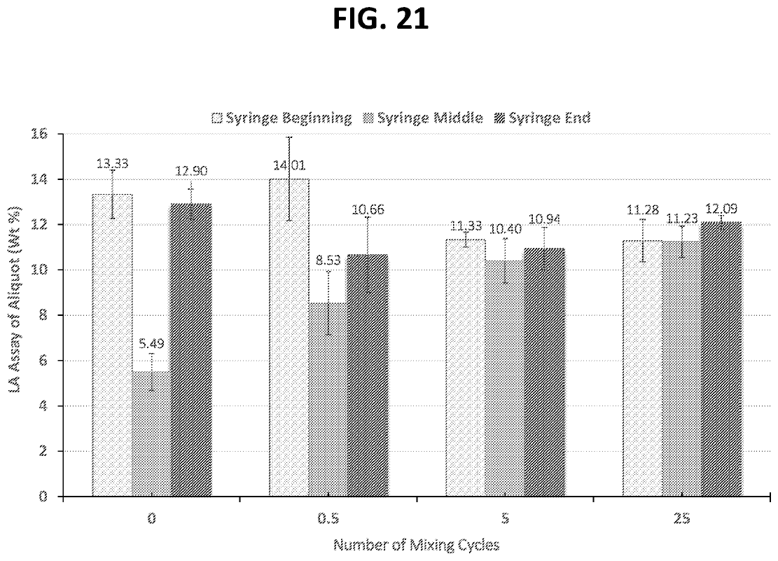

Sample aliquots were withdrawn from the beginning, middle, and end of the syringe (relative to the syringe tip) and delivered into a tared 25 mL volumetric flask to which 6 mL of acetonitrile:methanol at 50:50 ratio was subsequently added. These volumetric flasks were then mixed to fully dissolve the formulation sample aliquot and diluted with methanol:water at 35:65 ratio to make up the volume. Dissolved samples were then filtered through 0.2 μm PTFE filters and subjected to HPLC for LA content analysis. FIG. 21 shows the amount of LA collected from within each region of the mixing syringe after a certain number of mixing cycles. Following centrifuged simulated long-term storage, LA particles separated towards both ends of the syringe, which is different than the centrifugation and mixing studies with TU in Example 1. Before any re-mixing, the LA content within the beginning, middle, and end regions of the syringe (relative to the syringe tip) varied upon syringe fractionation analysis. While dose uniformity could be partially improved with re-mixing, dose inhomogeneity remained after 5 mixing cycles. Even after 25 mixing cycles, restoration of the original dose uniformity that was present prior to centrifuged simulated long-term storage could not be achieved to a desirable extent.

### Example 18: Preparation of a Temperature Sensitive High-Viscosity Poly(D,L-Caprolactone-Co-Lactide) Liquid Polymer Formulation Possessing Suitable Melt Characteristics

Similar to Example 2, liquid polymer formulations with temperature sensitive viscosity were prepared but using BnBzO in the solvent system. The preparation of a non-flowable, high viscosity formulation at low temperatures (i.e., ≤8° C.) which can convert to a flowable, low viscosity formulation upon warming to higher temperatures (i.e., ≥18° C.) for clinical administration, such that the API homogeneity is maintained within the syringe in order to ensure that a uniform API dose is delivered when administered without re-mixing, or with minimal re-mixing, the suspension was investigated.

To establish liquid polymer formulations with a temperature sensitive viscosity, a liquid polymer formulation comprising, by weight, 21.0% of 18.8 kDa 75:25 PDLCL acid-initiated liquid polymer, 39.0% of BnBzO and 40.0% of PEG 600 was prepared and observed for its physical state transition using visual observations. This formulation displayed a change in viscosity as evidenced by visual solidification when the formulations were cooled from room temperature to 5° C. The formulation exhibited solidification at low temperature and transitions into a flowable liquid within 15 minutes exposure to room temperature conditions. As a visual indication, this low temperature induced solidified formulation possessed high viscosity capable of maintaining the homogeneity of the initial suspension, which would remain unaltered when subjected to centrifugation.

To further characterize the freezing and melting characteristics of this formulation, DSC thermal analysis was conducted. DSC scans were collected by holding the sample at an initial temperature of 20° C. for 1 minute before being cooled to −20° C. at a rate of 10° C./minute. The sample was then held at −20° C. for 5 minutes before being warmed to 45° C. at a rate of 5° C./minute. Table 15 summarizes the results from the DSC thermal analysis. As a general, non-limiting consideration, potential formulations of interest may be identified as those which are generally: 1) substantially non-flowable with high viscosity at lower temperatures which may be demonstrated, for example, by a Melt Onset Temperature of about 5° C. or higher and 2) substantially flowable with low viscosity at higher temperatures which may be demonstrated by a Melt Peak Temperature of about 22° C. or less. However, in the case of these BnBzO solvent based formulations, the DSC analysis was not as sensitive for identifying suitable solidifying compositions, as it was for the previous examples with an NMP solvent based formulation. However, the DSC data does provide a quicker output on identifying the solidifying formulations compared to fractionation HPLC analysis as performed in Example 17. The DSC analysis combined with visual observation or other supporting data was used to identify suitable solidifying formulations

TABLE 15
DSC Melt Data Of PDLCL Acid-Initiated 18.8 kDa
Liquid Polymer Formulations With PEG 600

| | | | | | | Physical State Transition | | |
|---|---|---|---|---|---|---|---|---|
| | | | PEG | DSC Melt | DSC Melt | | | Transition |
| Formulation | Polymer | BnBzO | 600 | Onset | Peak | | | Time |
| No. | (%) | (%) | (%) | Temp (° C.) | Temp (° C.) | 5° C. | 25° C. | (min) |
| 1 | 21.0 | 39.0 | 40.0 | −6.94 | 4.90 | Solid | Liquid | 15 |

The DSC data shows that the Melt Onset and Melt Peak Temperatures are −6.94° C. and 4.90° C. respectively, which is lower than desired to qualify as a good solidifying formulation. However, the physical state transition visually observed indicates that the formulation was a solid at 5° C. or at lower temperatures and becomes a liquid after 15 minutes exposure to room temperature conditions or at 25° C., which makes it suitable to be a solidifying formulation.

### Example 19: Preparation of a Temperature Sensitive High-Viscosity Leuprolide Acetate Poly(D,L-Caprolactone-Co-Lactide) Liquid Polymer Formulation that Maintains LA-Polymer Suspension Homogeneity

A LA-liquid polymer formulation comprising, by weight, 12.0% LA, 18.5% of 18.8 kDa 75:25 PDLCL acid-initiated polymer, 34.3% BnBzO, and 35.2% PEG 600 was prepared to demonstrate the physical stability of suspension using simulated long-term storage with centrifugation model. The formulation was prepared and then subjected to “prolonged centrifuged simulated storage”, wherein it was pre-cooled to 4° C. before centrifuging at 2,000 rpm for 60 minutes. The LA dose uniformity was analyzed before and after being subjected to prolonged centrifuged simulated storage. As a comparative control, the dose uniformity data generated on a non-solidifying LA-liquid polymer formulation comprising, by weight, 12.0% LA, 30.8% of a 18 kDa 75:25 PDLCL acid-initiated polymer, and 57.2% BnBzO was used also analyzed. All formulations were analyzed in triplicate and none of the samples were re-mixed prior to LA dose uniformity analysis.

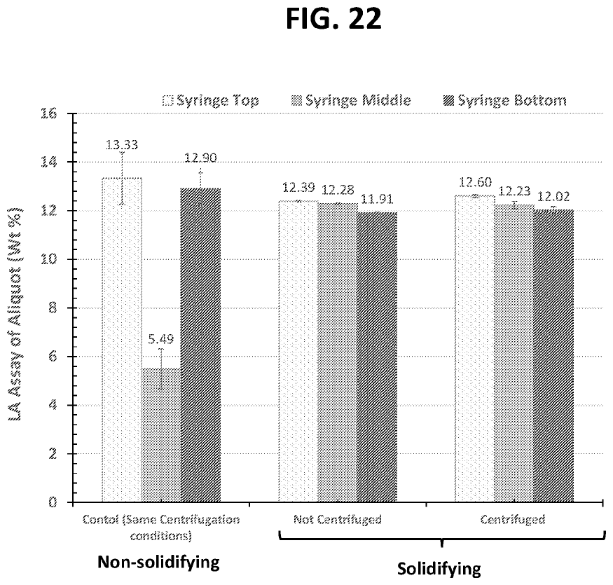

FIG. 22 shows the results of the LA dose uniformity for the analyzed samples. The dose uniformity of the non-solidifying LA-liquid polymer formulation experiences significant LA inhomogeneity upon prolonged centrifuged simulated storage. In contrast, the solidifying LA-liquid polymer formulation comprising additional PEG 600 does not undergo any significant LA inhomogeneity following simulated long-term storage.

The ability of PEG 600 to induce a low temperature sensitive high viscosity change within the LA-liquid polymer formulation is useful in maintaining the LA particle distribution within the liquid polymer phase when subjected to aggressive centrifugation to stimulate prolonged storage. These characteristics make this LA liquid-polymer formulation suitable for clinical use as it will be solidified at clinical storage conditions (i.e., refrigeration temperatures from about 0° C. to about 8° C.) but fully melted as a flowable formulation ready for administration by room temperature (i.e., at temperatures from about 18° C. to about 25° C.), as seen by visual observation and fractionation analysis. As such, a physician merely needs to remove a syringe with the solidified LA formulation from cold storage and allow it to equilibrate to room temperature before administering to a patient, such that the dose uniformity is well-maintained without the need to re-mix the now flowable liquid formulation.

### Example 20: Preparation Of Temperature Sensitive High-Viscosity Poly(D,L-Caprolactone-co-Lactide) Liquid Polymer Formulations

To establish temperature sensitive, high viscosity formulations comprising liquid polymers, formulations were prepared comprising, by weight, 14% or 34% of a 18.8 kDa 75:25 PDLCL acid-initiated liquid polymer with the addition of various amounts of BnBzO and PEG 600, and optionally PEG 1000, PEG 1450, or PEG 3350. In order to assess the amount of PEG 600 suitable for rendering the temperature-sensitive solidification properties as seen in the liquid polymer formulations in the previous examples, combinations of PEG 600 and one of PEG 1000, PEG 1450, or PEG 3350 were added to liquid polymer formulations. However, to accommodate the increasing amounts of PEG 600, the amounts of polymer and BnBzO were reduced correspondingly such that the % weight amount is totaled to 100% in the formulation composition.

To characterize the freezing and melting characteristics of these various formulations, DSC thermal analysis was conducted. DSC scans were conducted by holding the samples at an initial temperature of 20° C. for 1 minute before being cooled to −20° C. at a rate of 10° C./minute. Samples were then held at −20° C. for 10 minutes before being warmed to 50° C. at a rate of 5° C./minute. Suitable solidifying formulations were screened for those which were began to melt at about 5° C. or higher and which were fully melted as a flowable suspension by at about 20° C. or higher. Table 16 summarizes the results from the DSC thermal analysis for the various solid polymer formulations.

TABLE 16
DSC Melt Data and Visual Observation Data For Physical State Transition f 18.8 kDa PDLCL Acid-
Initiated Liquid Polymer Formulations With PEG 600 And Optionally Other Low Molecular Weight PEGs

| | | | | | | | DSC | DSC | | | |
|---|---|---|---|---|---|---|---|---|---|---|---|
| | | | | | | | Melt | Melt | | | |
| | | | PEG | PEG | PEG | PEG | Onset | Peak | Physical State Transition | | |
| Formulation | Polymer | BnBzO | 600 | 1000 | 1450 | 3350 | Temp | Temp | | | Transition |
| No. | (%) | (%) | (%) | (%) | (%) | (%) | (° C.) | (° C.) | 25° C. | 5° C. | Time (min.) |
| 1 | 14.0 | 26.0 | 60.0 | — | — | — | −0.71 | 9.93 | Solid | Liquid | 15 |
| 2 | 21.0 | 39.0 | 40.0 | — | — | — | −6.94 | 4.90 | Solid | Liquid | 15 |
| 3 | 19.3 | 35.8 | 40.0 | 5.0 | — | — | ND | ND | Solid | Liquid | 15 |
| 4 | 19.3 | 35.8 | 40.0 | — | 5.0 | — | ND | ND | Sample not available | | |
| 5 | 22.8 | 42.3 | 30.0 | — | 5.0 | — | ND | ND | Solid | Liquid | 30 |
| 6 | 25.9 | 48.1 | 25.0 | — | 1.0 | — | Two melt peaks | | Solid | Solid | Remained |
| 7 | 31.2 | 57.9 | 10.0 | — | — | 1.0 | Two melt peaks | | Solid | Solid | as solid |
| 8 | 32.9 | 61.1 | 5.0 | — | — | 1.0 | Two melt peaks | | Solid | Solid | after 30 |
| 9 | 33.6 | 62.4 | 2.0 | — | — | 2.0 | ND | ND | Solid | Solid | minutes |

*ND: Peaks not detected

Of the tested formulations, none of the formulations demonstrated suitable temperature transition states per DSC data criteria. However, the physical state transition observed by visual inspection determined that Test formulation Nos. 1-3, and 5 (Note: For test formulation 4, there was not enough sample quantity for visual observation) possessed suitable solidifying characteristics which make the formulations solidify at 5° C. or lower temperatures and transitions into liquid state at 18° C. or higher temperatures (generally at room temperature). Test formulation Nos. 6, 7, and 8 possessed two melt peaks in the DSC graphs (not shown) with right shift on temperature axis indicating that these three formulations are solid in nature with a physical state transition from solid to liquid that may need more time than the usual wait time. This observation is further supported by the visual observation data. The DSC profile of Test formulation No. 9 (not shown) has no peaks identified for freeze/melt profiles indicating that it is a non-suitable composition for the solidifying concept. However, the physical state transition data obtained by visual observation shows that the solid to liquid transition did not occur within 30 minutes at room temperature conditions, which may mean that the formulation is likely too solid to melt within a specific set time window for the user, i.e. generally of about 30 minutes at room temperature. However, this does show that the amount of PEG 600 used for suitable solidification properties can be as low as 2.0% when combined with PEG 3350 at 2.0%, when used in the liquid polymer formulations made of 18.8 kDa 75:25 PDLCL, acid-initiated polymer and BnBzO.

### Example 21: Preparation Of Temperature Sensitive High-Viscosity Poly(D,L-Caprolactone-co-Lactide) Liquid Polymer Formulations With PEG 1000

The results from Example 20 indicated that adding a higher molecular weight PEG, like PEG 3350, to the composition can be useful in reducing the amount of PEG 600 as a percentage of the solvent (i.e., compared to the formulation in Example 18). As different molecular weight PEGs possess different melting temperatures, it was hypothesized that a combination of higher and lower molecular weight PEGs, other than PEG 600, could also be used to further modulate the melt profiles of these BnBzO solvent based solidifying liquid polymer formulations.

PEG 1000 was selected for analysis to understand how the amount of a larger PEG effects solidification of the liquid polymer formulation. Increasing levels of PEG 1000 were used to make formulations with the pre-mixed polymer suspension in BnBzO comprising, by weight percentage, 35% of 75:25 PDLCL acid-initiated, 18.8 kDa and 65% BnBzO. Specifically, PEG 1000 was used in amounts of 2%, 5%, 10%, 20%, and 30%, by weight of the formulation. The formulations were prepared by adding the indicated quantities of PEG 1000 and the 35% polymer solution in BnBzO to a 4 mL clear scintillation vials, mixing the sample set at 50° C. on a Rotisserie mixer kept in oven maintaining 50°±2° C. temperature. The samples were completely melted, clear, and homogenous after 1 hour mixing at 50° C. The samples were stored in refrigerator (2-8° C.).

From visual observations, the addition of PEG 1000 in an amount as low as 5% to the liquid polymer formulation, resulted in solidification at refrigeration temperatures. Meanwhile, the addition of PEG 1000 in an amount of 30% leads to a firmer or thicker solid formation, which may require longer wait times for the formulation to become flowable at room temperature before administering the product to patient.

DSC melt analysis of these samples was performed. The DSC data and visual observation data of these formulations are provided in Table 17 below.

TABLE 17
DSC Melt Data And Visual Observation Data For Physical State Transition Of
PDLCL Acid-Initiated 18.8 kDa Liquid Polymer Formulations With PEG 1000

| | | | | DSC Melt | DSC Melt | | | |
|---|---|---|---|---|---|---|---|---|
| | | | PEG | Onset | Peak | Physical State Transition | | |
| Formulation | Polymer | BnBzO | 1000 | Temp | Temp | | | Transition |
| No. | (%) | (%) | (%) | (° C.) | (° C.) | 5° C. | 25° C. | Time (min.) |
| 1 | 34.3 | 63.7 | 2.0 | ND | ND | Liquid | Liquid | 0 |
| 2 | 33.3 | 61.8 | 5.0 | ND | ND | Solid | Liquid | 15 |
| 3 | 31.5 | 58.5 | 10.0 | 2.18 | 13.99 | Solid | Liquid | 15 |
| 4 | 28.0 | 52.0 | 20.0 | 7.61 | 19.31 | Solid | Liquid | 30 |
| 5 | 24.5 | 45.5 | 30.0 | 9.39 | 22.56 | Solid | Solid | Solid after |
| | | | | | | | | 30 min |

*ND: Peak not detected

Increasing amounts of PEG 1000 added to the liquid polymer formulations corresponded with an upward shift of the DSC Melt Onset and Melt Peak Temperatures. When compared to the melt profile of a solidifying liquid polymer formulation containing PEG 600, the addition of PEG 1000 at an amount of 20% to 30% of the formulation demonstrated a suitable Melt Onset Temperature and Melt Peak Temperature ranges similar to that seen for the solidifying liquid polymer formulation Example 2. However, physical state transition data from visual observation indicates that the addition of PEG 1000 at an amount of 5% or higher to liquid polymer solution in BnBzO achieves the solidifying properties, which makes it good enough to achieve and maintain homogeneity of API in a suspension.

### Example 22: Preparation Of A Temperature Sensitive High-Viscosity Leuprolide Acetate Poly(D,L-Caprolactone-co-Lactide) Liquid Polymer Formulation using Two Different Levels of PEG 1000 That Maintains LA-Polymer Suspension Homogeneity

To demonstrate the solidifying characteristics of formulations presented in Example 21, test formulations 2 and 4 were repeated, but with LA present in the formulation. The homogeneity of the various syringe fractions was then performed. The formulation details are presented in Table 18 below.

TABLE 18
PDLCL Acid-Initiated 18.8 kDa Liquid Polymer Formulations With PEG
1000

| Formulation No. | LA (%) | Polymer (%) | PEG 1000 (%) | BnBzO (%) |
|---|---|---|---|---|
| 1 | 12.0 | 29.3 | 4.4 | 54.3 |
| 2 | 12.0 | 24.6 | 17.6 | 45.8 |

In each case, the formulation was subjected to “prolonged centrifuged simulated storage”, wherein it was cooled to 4° C. and then centrifuged at 2,000 rpm for 60 minutes. The LA dose uniformity was analyzed before and after being subjected to prolonged centrifuged simulated storage. The dose uniformity data generated on non-solidifying LA-liquid polymer formulation comprising, by weight, 12.0% LA, 30.8% of a 18 kDa 75:25 PDLCL acid-initiated polymer, and 57.2% BnBzO was used as control. All formulations were analyzed in triplicate and none of the samples were re-mixed prior to LA dose uniformity analysis.

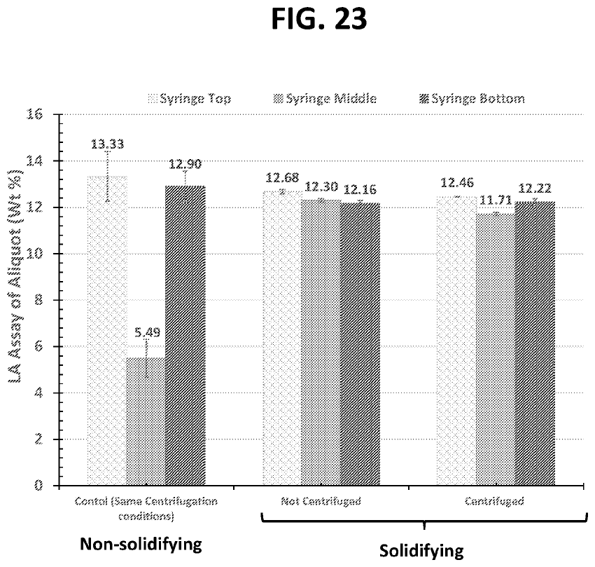

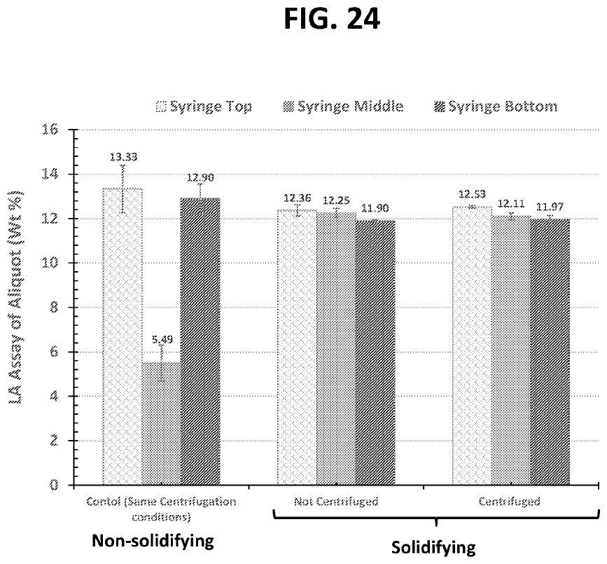

FIGS. 23 and 24 show the results of the LA dose uniformity for formulation Nos. 1 and 2 in Table 18, respectively, along with the control formulation. The dose uniformity of the non-solidifying control LA-liquid polymer formulation experiences significant LA inhomogeneity upon prolonged centrifuged simulated storage. In contrast, the solidifying LA-liquid polymer formulations comprising additional PEG 1000 at 4.4% and 17.6% levels respectively did not undergo any significant LA inhomogeneity following being subject to centrifugation. The ability of PEG 1000 to induce a low temperature sensitive high viscosity state within the LA-liquid polymer formulation is useful in maintaining the LA particle distribution within the liquid polymer formulation (i.e., suspension) when subsequently subjected to aggressive centrifugation to simulate prolonged storage. The data demonstrates that the effective concentration of PEG 1000 can be as low as 4.4% when used in a liquid polymer formulation.

### Example 23: Preparation Of Temperature Sensitive High-Viscosity Poly(D,L-Lactide-co-Glycolide)-Liquid Polymer Formulations With PEG 1450 Added After Formulation

PEG 1450 was selected for further analysis to understand how the amount of larger PEG effects solidification of the liquid polymer formulation. Increasing amounts of PEG 1450 was added to 4 mL scintillation vials containing a pre-mixed liquid polymer formulation comprising, by weight percentage, 35% of an 18.8 kDa 75:25 PDLCL acid-initiated polymer and 65% of BnBzO. Specifically, PEG 1450 content from 2% and 20%, by weight was used to make the formulations and the formulations were mixed as described in the previous examples.

From visual observations, the addition of PEG 1450 at the lowest tested amount of 2% of the formulation resulted in solidification at refrigeration temperatures. DSC analysis and visual observation of these samples were performed. The DSC data (not shown) and observed visual appearance of these formulations are provided in Table 19 below.

TABLE 19
DSC Melt And Visual Observation Data Visual Observation Data For Physical State Transition
Of PDLCL Acid-Initiated 18.8 kDa Liquid Polymer Formulations With PEG 1450

| | | | | DSC Melt | DSC Melt | | | |
|---|---|---|---|---|---|---|---|---|
| | | | | Onset | Peak | Physical State Transition | | |
| Formulation | Polymer | BnBzO | PEG1450 | Temp | Temp | | | Transition |
| No. | (%) | (%) | (%) | (° C.) | (° C.) | 5° C. | 25° C. | Time (min.) |
| 1 | 34.3 | 63.7 | 2.0 | ND | ND | Solid | Liquid | 15 |
| 2 | 33.3 | 61.8 | 5.0 | ND | ND | Solid | Liquid | 30 |
| 3 | 31.5 | 58.5 | 10.0 | 15.28 | 25.49 | Solid | Solid | Solid after |
| | | | | | | | | 30 min |
| 4 | 28.0 | 52.0 | 20.0 | 16.10 | 28.47 | Solid | Solid | Solid after |
| | | | | | | | | 30 min |

*ND: Peak not detected

Increasing amounts of PEG 1450 added to the liquid polymer formulations corresponded with an upward shift of the Peak Onset and Peak Melt Temperatures. When compared to the melt profile of a solidifying liquid polymer formulation containing PEG 600, the addition of PEG 1450 at 10% to 20% of the liquid polymer formulation demonstrated a Melt Onset Temperature and Melt Peak Temperature range slightly above that of the reference solidifying liquid polymer formulation. When the amount of PEG 1450 was at 10% of the formulation, the Melt Onset Temperature and Melt Peak Temperature occurred at 15.28° C. and 25.49° C., respectively.

### Example 24: Preparation Of Temperature Sensitive High-Viscosity Poly(D,L-Lactide-co-Glycolide) Liquid Polymer Formulations With PEG 3350 Added After Formulation

PEG 3350 was selected for further analysis to understand how the amount of larger PEG effects solidification of the liquid polymer formulation. Increasing amounts of PEG 3350 was added to scintillation vials containing a pre-mixed liquid polymer formulation comprising, by weight percentage, 35% of an 18.8 kDa 75:25 PDLCL acid-initiated polymer and 65% of BnBzO. Specifically, PEG 3350 content from 1% and 15%, by weight were used to make the formulations and the formulations were mixed as described in the previous examples.

From visual observations, addition of PEG 3350 at the lowest tested amount of 1% of the formulation resulted in solidification at refrigeration temperatures. DSC analysis and visual observation of these samples were performed. The DSC data and observed visual appearance of these formulations are provided in Table 20 below.

TABLE 20
DSC Melt And Visual Observation Data Visual Observation Data For Physical State Transition
Of PDLCL Acid-Initiated 18.8 kDa Liquid Polymer Formulations With PEG 3350

| | | | | DSC Melt | DSC Melt | | | |
|---|---|---|---|---|---|---|---|---|
| | | | PEG | Onset | Peak | Physical State Transition | | |
| Formulation | Polymer | BnBzO | 3350 | Temp | Temp | | | Transition |
| No. | (%) | (%) | (%) | (° C.) | (° C.) | 5° C. | 25° C. | Time (min.) |
| 1 | 64.4 | 34.7 | 1.0 | ND | ND | Solid | Liquid | 30 |
| 2 | 63.7 | 34.3 | 2.0 | 24.73 | 27.89 | Solid | Solid | Solid after |
| 3 | 61.8 | 33.3 | 5.0 | 23.62 | 28.32 | Solid | Solid | 30 min |

| | | | | | | |
|---|---|---|---|---|---|---|
| 4 | 58.5 | 31.5 | 10.0 | Two melt peaks | Solid | Solid |
| 5 | 55.3 | 29.8 | 15.0 | Two melt peaks | Solid | Solid |

*ND: Peak not detected

Increasing amounts of PEG 3350 added to the liquid polymer formulations corresponded with an upward shift of the Peak Onset and Peak Melt Temperatures. When compared to the melt profile of a solidifying liquid polymer formulation containing PEG 600, the addition of PEG 3350 at 1% to 15% of the liquid polymer formulation demonstrated a Melt Onset Temperature and Melt Peak Temperature range slightly above that of the reference solidifying liquid polymer formulation. From the visual observation, it is clear that PEG 3350 is effective at 1% concentration when used in liquid polymer formulation resulting into solidification properties, which need about 30 minutes transition time before the sample liquefies at room temperature. PEG 3350, at content, 2% and higher shows a firmer solid formation which may need more than 30 minutes wait time before sample transitions from solid to liquid.

1. A pharmaceutical extended release composition, comprising:
an active pharmaceutical ingredient; and
a biocompatible polymer-solvent system comprising a biodegradable polymer and a solvent system comprising at least one solvent and at least one component that modifies the melting point of the polymer-solvent system to:
form a highly viscous composition that maintains a substantially homogeneous distribution of the active pharmaceutical ingredient at a first temperature from about 0° C. to about 8° C.; and
form a flowable composition suitable for administration by injection at a second temperature from about 18° C. to about 25° C.

2. The composition of claim 1, wherein the first temperature is from about 2° C. to about 6° C., and the second temperature is from about 20° C. to about 24° C.

3-4. (canceled)

5. The composition of claim 1, wherein the solvent system comprises one or more solvents selected from the group consisting of N-methyl-2-pyrrolidone (NMP), acetone, cyrene, butyrolactone, ε-caprolactone, N-cycylohexyl-2-pyrrolidone, diethylene glycol monomethyl ether, dimethylacetamide, dimethyl formamide, dimethyl sulfoxide (DMSO), ethyl acetate, ethyl lactate,N-ethyl-2-pyrrolidone, glycerol formal, glycofurol, N-hydroxyethyl-2-pyrrolidone,isopropylidene glycerol, lactic acid, methoxypolyethylene glycol, methoxypropyleneglycol, methyl acetate, methyl ethyl ketone, methyl lactate, benzyl benzoate (BnBzO), polysorbate 80, polysorbate 60, polysorbate 40, polysorbate 20, polyoxyl 35, polyethylene glycol (PEG), hydrogenated castor oil, polyoxyl 40 hydrogenated castor oil, sorbitan monolaurate, sorbitan monostearate, sorbitan monooleate, benzyl alcohol, n-propanol, isopropanol, tert-butanol, propylene glycol, 2-pyrrolidone, a-tocopherol, triacetin, tributyl citrate, acetyl tributyl citrate, acetyl triethyl citrate, triethyl citrate, esters thereof, and combinations thereof.

6. (canceled)

7. The composition of claim 1, wherein the solvent system comprises at least one solvent and a co-solvent selected from a low molecular weight PEG having a number average molecular weight of PEG 300, PEG 400, or a combination thereof, wherein the low molecular weight PEG does not modify the melting point of the biocompatible polymer-solvent system.

8. (canceled)

9. The composition of claim 1, wherein the at least one component comprises at least one low molecular weight PEG selected from the group consisting of PEG 500, PEG 600, PEG 1000, PEG 1450, PEG 3350, and combinations thereof.

10. (canceled)

11. The composition of claim 9, wherein a weight % ratio of the at least one low molecular weight PEG to the biocompatible polymer-solvent system is from about 1:20 to about 20:1.

12. The composition of claim 9, wherein the composition comprises:
from about 1 wt % to about 90 wt % of the at least one low molecular weight PEG;
from about 15 wt % to about 70 wt % of PEG 600;
from about 4 wt % to about 45 wt % of PEG 1000;
from about 2 wt % to about 35 wt % of PEG 1450; or
from about 1 wt % to about 10 wt % of PEG 3350.

13-16. (canceled)

17. The composition of claim 1, wherein the biodegradable polymer is selected from the group consisting of polylactic acid, polyglycolic acid, polylactide, polyglycolide, polycaprolactones, polyanhydrides, polyamides, polyurethanes, polyesteramides, polyorthoesters, polydioxanones, polyacetals, polyketals, polycarbonates, polyphosphazenes, polyhydroxybutyrates, polyhydroxyvalerates, polyalkylene oxalates, polyalkylene succinates, poly(malic acid), polyethylene glycol, hyaluronic acid, chitin and chitosan, a copolymer thereof, a terpolymer thereof, and combinations thereof.

18. (canceled)

19. The composition of claim 1, wherein the biodegradable polymer comprises lactide and glycolide monomer residues in a molar ratio of the lactide to glycolide monomer residues selected from about 45:55 to about 99:1 and about 50:50 to about 90:10.

20-21. (canceled)

22. The composition of claim 1, wherein the biodegradable polymer comprises:
lactide and/or glycolide monomer residues; and
monomer residues selected from the group consisting of ε-caprolactone, trimethylene carbonate, and combinations thereof;
in a molar ratio of the lactide and/or glycolide monomer residues to the ε-caprolactone and/or trimethylene carbonate monomer residues selected from about 10:90 to about 90:10, from about 25:75 to about 75:25, and 75:25.

23-25. (canceled)

26. The composition of claim 19, wherein:
the biodegradable polymer comprises at least one carboxylic acid end group is synthesized by initiation with an organic acid;
the biodegradable polymer comprises at least one hydroxy end group and is synthesized by initiation with a mono functional alcohol; or
the biodegradable polymer comprises at least one hydroxy end group, is substantially free of terminal carboxy end groups, and is synthesized by initiation with a diol.

27-28. (canceled)

29. The composition of claim 1, wherein the biodegradable polymer has an average molecular weight selected from about 1 kDa to about 100 kDa, and about 1 kDa to about 60 kDa.

30. (canceled)

31. The composition of claim 1, wherein the biodegradable polymer is not soluble in water.

32. The composition of claim 1, wherein the composition comprises from about 0.1 wt % to about 70 wt % of the biodegradable polymer, from about 1 wt % to about 70 wt % of the biodegradable polymer, or from about 10 wt % to about 50 wt % of the biodegradable polymer.

33-42. (canceled)

43. The composition of claim 1, wherein the composition is suitable for administration by injection or auto injection at a temperature of about 18° C. or more.

44-45. (canceled)

46. The composition of claim 1, wherein the composition has a viscosity at the first temperature selected from the group consisting of about 20,000 cP or more, about 10,000 cP or more, about 5,000 cP or more, about 20,000 cP or less, about 10,000 cP or less, and about 5,000 cP or less.

47-51. (canceled)

52. The composition of claim 1, wherein the composition maintains a substantially homogeneous distribution of the active pharmaceutical ingredient within the composition when stored at the first temperature for at least 6 months, at least 12 months, at least 24 months, at least 36 months, or longer.

53-55. (canceled)

56. The composition of claim 1, wherein the composition is a liquid-liquid dispersion, a liquid-in-oil dispersion, or an emulsion.

57-58. (canceled)

59. The composition of claim 1, wherein the composition is stored at the first temperature and then is warmed to the second temperature prior to administering to a subject.

60-61. (canceled)

62. The composition of claim 1, wherein upon contact of the composition with a bodily fluid, a solvent dissipates and an in situ liquid or solid implant forms.

63-67. (canceled)

68. A delivery system for administration of a pharmaceutical composition, comprising:
a syringe; and
the pharmaceutical composition of claim 1,
wherein the pharmaceutical composition is contained within the syringe.

69. (canceled)
